(19) United States
(12) Patent Application Publication
(10) Pub. No.: US 2024/0209099 A1
(43) Pub. Date: Jun. 27, 2024

(54) NOVEL ANTI-HVEGFR2 ANTIBODIES

(71) Applicant: SUZHOU TRANSCENTA THERAPEUTICS CO., LTD., Suzhou, Jiangsu (CN)

(72) Inventors: Xueming QIAN, Suzhou, Jiangsu (CN); Zhenping LI, Suzhou, Jiangsu (CN); Hongjun LI, Suzhou, Jiangsu (CN); Huanhuan GUO, Suzhou, Jiangsu (CN)

(21) Appl. No.: 18/555,341

(22) PCT Filed: Apr. 13, 2022
(86) PCT No.: PCT/CN2022/086525

§ 371 Date: Oct. 13, 2023

(30) Foreign Application Priority Data
Apr. 14, 2021 (WO) PCT/CN2021/087278

(51) Int. Cl. C07K 16/28; A61K 39/00; A61K 47/68; A61K 49/00; A61K 51/10; A61P 35/00; G01N 33/68

(52) U.S. Cl. CPC C07K 16/2863; A61K 47/6849; A61K 49/0058; A61K 51/103; A61P 35/00; G01N 33/6872; A61K 2039/505; C07K 2317/24; C07K 2317/31; G01N 2333/475

(57) ABSTRACT

Provided are anti-hVEGFR2 antibodies or antigen-binding fragments thereof, isolated polynucleotides encoding the same, pharmaceutical compositions comprising the same, and the uses thereof.

### FIELD OF THE INVENTION

The present disclosure generally relates to novel anti-hVEGFR2 antibodies that specifically bind to human VEGFR2 (hVEGFR2).

### BACKGROUND

Vascular endothelial growth factor receptor 2 (VEGFR2) is a type III tyrosine kinase that can be stimulated by vascular endothelial growth factor (VEGF) (e.g., VEGF-A, VEGF-C and VEGF-D). Upon binding to VEGF-A, VEGFR2 increases its expression and becomes dimerized form. The dimerization of VEGFR2 induces tyrosine phosphorylation of VEGFR2 followed by activation of downstream pathways involved in proliferation, migration, differentiation, tube formation, maintenance of vascular integrity and increase in vascular permeability of the endothelial cells. Abnormally high expression and/or activity of VEGFR2 disrupts the homeostasis of angiogenesis and leads to various cancers and/or angiogenic diseases.

Therefore, there exists significant needs for novel anti-hVEGFR2 antibodies which can be used for treatment of diseases positive for hVEGFR2 expression, such as cancers as well as other angiogenic diseases.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

The present disclosure provides, among others, novel monoclonal anti-hVEGFR2 antibodies, nucleotide sequences encoding such, and the uses thereof.

In one aspect, the present disclosure provides an anti-hVEGFR antibody or an antigen-binding fragment thereof, comprising heavy chain HCDR1, HCDR2 and HCDR3 and/or light chain LCDR1, LCDR2 and LCDR3 sequences, wherein:
the HCDR1 sequence comprises SSWMN (SEQ ID NO: 1), DYYMS (SEQ ID NO: 19), X₁YGMS (SEQ ID NO: 41), X₄YWIM (SEQ ID NO: 44), or a homologue sequence of at least 80% sequence identity thereof,
the HCDR2 sequence comprises RIFPGDGDTYYNGKFQV (SEQ ID NO: 2), FIRNKANGYTTEYSASVKG (SEQ ID NO: 20), SISX₂GGSYTYYADSVX₁₉G (SEQ ID NO: 42), DIYPGX₅GSTNYNEKFKS (SEQ ID NO: 45) or a homologue sequence of at least 80% sequence identity thereof,
the HCDR3 sequence comprises FLDTSGRYVDY (SEQ ID NO: 3), FDYYGSTYCFDY (SEQ ID NO: 21), EX₃DGNYDY (SEQ ID NO: 43), DSNPDY (SEQ ID NO: 46), or a homologue sequence of at least 80% sequence identity thereof;
the LCDR1 sequence comprises KASQDVNTAVA (SEQ ID NO: 4), RASQSVSTSSSSFMH (SEQ ID NO: 22), RSSKSLLYKDGKTYLN (SEQ ID NO: 28), RASESVX₆NSGISFMX₇ (SEQ ID NO: 47) or a homologue sequence of at least 80% sequence identity thereof,
the LCDR2 sequence comprises SASYRYI (SEQ ID NO: 5), YASNLES (SEQ ID NO: 23), LMSTRAS (SEQ ID NO: 29), AASX₈QX₉S (SEQ ID NO: 48) or a homologue sequence of at least 80% sequence identity thereof;
the LCDR3 sequence comprises QQHYRAPLT (SEQ ID NO: 6), QHTWEIPLT (SEQ ID NO: 24), QQLVEYPFT (SEQ ID NO: 30), QQSKEVPYT (SEQ ID NO: 49) or a homologue sequence of at least 80% sequence identity thereof;
wherein X1 is I or M, X₂ is V or I, X₃ is L or M, X₄ is T or S, X₅ is T or S, X₆ is D or E, X₇ is T or H, X₈ is T or Y, X₉ is G or R, and X₁₉ is E or K.

In certain embodiments, the HCDR1 comprises an amino acid sequence of SEQ ID NO: 41, the HCDR2 comprises an amino acid sequence of SEQ ID NO: 42, the HCDR3 comprises an amino acid sequence of SEQ ID NO: 43, the LCDR1 comprises a sequence of SEQ ID NO: 28, the LCDR2 comprises a sequence of SEQ ID NO: 29, and the LCDR3 comprises a sequence of SEQ ID NO: 30.

In certain embodiments, in the antibody or an antigen-binding fragment thereof provided herein, wherein
a) the HCDR1 comprises the sequence of SEQ ID NO: 25, a HCDR2 comprises the sequence of SEQ ID NO: 26, the HCDR3 comprises the sequence of SEQ ID NO: 27; the LCDR1 comprises the sequence of SEQ ID NO: 28, the LCDR2 comprises the sequence of SEQ ID NO: 29, and the LCDR3 comprises the sequence of SEQ ID NO: 30; or
b) the HCDR1 comprises the sequence of SEQ ID NO: 31, the HCDR2 comprises the sequence of SEQ ID NO: 32 or SEQ ID NO: 37, and the HCDR3 comprises the sequence of SEQ ID NO: 33, the LCDR1 comprises the sequence of SEQ ID NO: 28, the LCDR2 comprises the sequence of SEQ ID NO: 29, and the LCDR3 comprises the sequence of SEQ ID NO: 30; or
c) the HCDR1 comprises the sequence of SEQ ID NO: 34, the HCDR2 comprises the sequence of SEQ ID NO: 35 or SEQ ID NO: 37, and the HCDR3 comprises the sequence of SEQ ID NO: 36, the LCDR1 comprises the sequence of SEQ ID NO: 28, the LCDR2 comprises the sequence of SEQ ID NO: 29, and the LCDR3 comprises the sequence of SEQ ID NO: 30.

In certain embodiments, in the anti-hVEGFR antibody or an antigen-binding fragment thereof provided herein, wherein the HCDR1 comprises an amino acid sequence of SEQ ID NO: 44, the HCDR2 comprises an amino acid sequence of SEQ ID NO: 45, the HCDR3 comprises an amino acid sequence of SEQ ID NO: 46, the LCDR1 comprises a sequence of SEQ ID NO: 47, the LCDR2 comprises a sequence of SEQ ID NO: 48, and the LCDR3 comprises a sequence of SEQ ID NO: 49.

In certain embodiments, in the antibody or an antigen-binding fragment thereof provided herein, wherein
a) the HCDR1 comprises the sequence of SEQ ID NO: 7, a HCDR2 comprises the sequence of SEQ ID NO: 8, the HCDR3 comprises the sequence of SEQ ID NO: 9; the LCDR1 comprises the sequence of SEQ ID NO: 10, the LCDR2 comprises the sequence of SEQ ID NO: 11, and the LCDR3 comprises the sequence of SEQ ID NO: 12; or
b) the HCDR1 comprises the sequence of SEQ ID NO: 13, the HCDR2 comprises the sequence of SEQ ID NO: 14, and the HCDR3 comprises the sequence of SEQ ID NO: 15, the LCDR1 comprises the sequence of SEQ ID NO: 16, the LCDR2 comprises the sequence of SEQ ID NO: 17, and the LCDR3 comprises the sequence of SEQ ID NO: 18.

In certain embodiments, in the antibody or an antigen-binding fragment thereof provided herein, wherein
a) the HCDR1 comprises the sequence of SEQ ID NO: 1, the HCDR2 comprises the sequence of SEQ ID NO: 2, and the HCDR3 comprises the sequence of SEQ ID NO: 3, the LCDR1 comprises the sequence of SEQ ID NO: 4, the LCDR2 comprises the sequence of SEQ ID NO: 5, and the LCDR3 comprises the sequence of SEQ ID NO: 6; or
b) the HCDR1 comprises the sequence of SEQ ID NO: 19, the HCDR2 comprises the sequence of SEQ ID NO: 20, and the HCDR3 comprises the sequence of SEQ ID NO: 21, the LCDR1 comprises the sequence of SEQ ID NO: 22, the LCDR2 comprises the sequence of SEQ ID NO: 23, and the LCDR3 comprises the sequence of SEQ ID NO: 24.

In certain embodiments, the antibody or an antigen-binding fragment thereof provided herein further comprises one or more of heavy chain HFR1, HFR2, HFR3 and HFR4, and/or one or more of light chain LFR1, LFR2, LFR3 and LFR4, wherein:
the HFR1 comprises EVQLVESGGGLVKPGGSLXioLSCAASGFTFS (SEQ ID NO: 84) or a homologous sequence of at least 80% (or at least 85%, 90%, 95%) sequence identity thereof,
the HFR2 comprises WVRQX₁₁PGKRLEWVA (SEQ ID NO: 85) or a homologous sequence of at least 80% (or at least 90%) sequence identity thereof,
the HFR3 sequence comprises RFTISRDNAKNTLYLQMNSLX₁₂AEDTAVYYCAR (SEQ ID NO: 86) or a homologous sequence of at least 80% (or at least 85%, 90%, 95%) sequence identity thereof,
the HFR4 comprises WGX₁₃GTTLTVSS (SEQ ID NO: 87) or a homologous sequence of at least 80% sequence identity thereof,
the LFR1 comprises DIVITQX₁₄X₁₅LSLPVTX₁₆GESVSISC (SEQ ID NO: 88) or a homologous sequence of at least 80% (or at least 85%, 90%, 95%) sequence identity thereof,
the LFR2 comprises WFLQRPGQSPQLLIY (SEQ ID NO: 89) or a homologous sequence of at least 80% (or at least 85%, 90%) sequence identity thereof,
the LFR3 comprises GVX₁₇DRFSGSGSGTDFTLKISRVEAEDVGX₁₈YYC (SEQ ID NO: 90) or a homologous sequence of at least 80% (or at least 85%, 90%, 95%) sequence identity thereof, and
the LFR4 comprises FGSGTKLEIK (SEQ ID NO: 91) or a homologous sequence of at least 80% (or at least 90%) sequence identity thereof, wherein X₁₀ is R or K, X₁₁ is A or T, X₁₂ is R or K, X₁₃ is Q or H, X₁₄ is D or T, X₁₅ is E or P, X₁₆ is F or P, X₁₇ is S or P, X₈ is V or I.

In certain embodiments, in the antibody or antigen-binding fragment thereof provided herein, wherein:
the HFR1 comprises a sequence selected from the group consisting of SEQ ID NOs: 64, 68 and 72,
the HFR2 comprises a sequence selected from the group consisting of SEQ ID NOs: 65, 69 and 73,
the HFR3 comprises the sequence selected from the group consisting of SEQ ID NOs: 66, 70 and 74,
the HFR4 comprises a sequence selected from the group consisting of SEQ ID NOs: 67, 71 and 75,
the LFR1 comprises the sequence selected from the group consisting of SEQ ID NOs: 76 and 80,
the LFR2 comprises a sequence selected from the group consisting of SEQ ID NO: 77 and 81,
the LFR3 comprises a sequence selected from the group consisting of SEQ ID NOs: 78 and 82, and
the LFR4 comprises a sequence selected from the group consisting of SEQ ID NOs: 79 and 83.

In certain embodiments, in the antibody or an antigen-binding fragment thereof provided herein, wherein the heavy chain variable region comprises a sequence selected from the group consisting of SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 93, SEQ ID NO: 94, SEQ ID NO: 98, and a homologous sequence thereof having at least 80% sequence identity yet retaining specific binding affinity to hVEGFR2.

In certain embodiments, in the antibody or an antigen-binding fragment thereof provided herein, wherein the light chain variable region comprises a sequence selected from the group consisting of SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 96, SEQ ID NO: 97, and a homologous sequence thereof having at least 80% sequence identity yet retaining specific binding affinity to hVEGFR2.

In certain embodiments, in the antibody or an antigen-binding fragment thereof provided herein, wherein:
a) a heavy chain variable region comprising the sequence of SEQ ID NO: 50 and a light chain variable region comprising the sequence of SEQ ID NO: 51;
b) the heavy chain variable region comprises a sequence of SEQ ID NO: 52 and the light chain variable region comprises a sequence of SEQ ID NO: 53;
c) the heavy chain variable region comprises a sequence of SEQ ID NO: 54 and the light chain variable region comprises a sequence of SEQ ID NO: 55;
d) the heavy chain variable region comprises a sequence of SEQ ID NO: 56 and the light chain variable region comprises a sequence of SEQ ID NO: 57;
e) the heavy chain variable region comprises a sequence of SEQ ID NO: 58 and the light chain variable region comprises a sequence of SEQ ID NO: 59;
f) the heavy chain variable region comprises a sequence of SEQ ID NO: 60 and the light chain variable region comprises a sequence of SEQ ID NO: 61;
g) the heavy chain variable region comprises a sequence of SEQ ID NO: 62 and the light chain variable region comprises a sequence of SEQ ID NO: 63; or
h) the heavy chain variable region comprises a sequence of SEQ ID NO: 93 or SEQ ID NO: 94 or SEQ ID NO: 98 and the light chain variable region comprises a sequence of SEQ ID NO: 96 or SEQ ID NO: 97.

In certain embodiments, the antibody or antigen-binding fragment thereof provided herein further comprises one or more amino acid residue substitutions or modifications yet retains specific binding affinity to hVEGFR2.

In certain embodiments, at least one of the substitutions or modifications is in one or more of the CDR sequences, and/or in one or more of the non-CDR regions of the VH or VL sequences.

In certain embodiments, the antibody or antigen-binding fragment provided herein further comprises an immunoglobulin constant region, optionally a constant region of human Ig, or optionally a constant region of human IgG.

In certain embodiments, the constant region comprises a constant region of human IgG1, IgG2, IgG3, or IgG4.

In certain embodiments, the constant region of human IgG1 comprises SEQ ID NO: 38, or a homologous sequence having at least 80% sequence identity thereof.

In certain embodiments, the antibody or an antigen-binding fragment thereof provided herein is humanized.

In certain embodiments, the antibody or antigen-binding fragment thereof provided herein is a diabody, a Fab, a Fab′, a F(ab′)2, a Fd, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

In certain embodiments, the antibody or antigen-binding fragment thereof provided herein is bispecific.

In certain embodiments, the antibody or antigen-binding fragment thereof provided herein is capable of specifically binding to a first and a second epitope of hVEGFR2, or capable of specifically binding to hVEGFR2 and a second antigen.

In certain embodiments, the second antigen is an immune related target, optionally selected from the group consisting of. PD-L1, PD-L2, PD-1, CLTA-4, TIM-3, LAG3, CD160, 2B4, TGFβ, VISTA, BTLA, TIGIT, LAIR1, OX40, CD2, CD27, ICAM-1, NKG2C, SLAMF7, NKp80, CD160, B7-H3, LFA-1, 1COS, 4-1BB, GITR, CD30, CD40, BAFFR, HVEM, CD7, LIGHT, IL-2, IL-15, CD3, CD16 and CD83.

In certain embodiments, the second antigen comprises a tumor antigen.

In certain embodiments, the tumor antigen is present in a VEGFR2-expressing cell.

In certain embodiments, the tumor antigen comprises claudin 18.2, CA-125, gangliosides G(D2), G(M2) and G(D3), CD20, CD52, CD33, Ep-CAM, CEA, bombesin-like peptides, PSA, HER2/neu, epidermal growth factor receptor (EGFR), erbB2, erbB3/HER3, erbB4, CD44v6, Ki-67, cancer-associated mucin, VEGF, VEGFRs (e.g., VEGFR3), estrogen receptors, Lewis-Y antigen, TGFβ1, IGF-1 receptor, EGFα, c-Kit receptor, transferrin receptor, IL-2R or CO17-1A.

In certain embodiments, the antibody or antigen-binding fragment thereof provided herein is linked to one or more conjugate moieties.

In certain embodiments, the conjugate moiety comprises a clearance-modifying agent, a chemotherapeutic agent, a toxin, a radioactive isotope, a lanthanide, a luminescent label, a fluorescent label, an enzyme-substrate label, a DNA-alkylators, a topoisomerase inhibitor, a tubulin-binder, or other anticancer drugs.

In another aspect, the present disclosure also provides an antibody or an antigen-binding fragment thereof that competes for binding to hVEGFR2 with the antibody or antigen-binding fragment provided herein.

In one aspect, the present disclosure provides a pharmaceutical composition comprising the antibody or antigen-binding fragment provided herein, and one or more pharmaceutically acceptable carriers.

In certain embodiments, the pharmaceutical composition provided herein further comprises a second therapeutic agent.

In certain embodiments, the second therapeutic agent comprises an anti-cancer therapy, optionally the anti-cancer therapy is selected from a chemotherapeutic agent, radiation therapy, an immunotherapy agent, anti-angiogenesis agent (e.g. antagonist of a VEGFR such as VEGFR-1, and VEGFR-3), an EGFR antagonist, an PDGFR antagonist, an IGFR antagonist, an NGFR antagonist, an FGFR antagonist, a targeted therapy agent (e.g. HER2 antibody, claudin 18.2 antibody), a cellular therapy agent, a gene therapy agent, a hormonal therapy agent, cytokines, palliative care, surgery for the treatment of cancer (e.g., tumorectomy), one or more anti-emetics, treatments for complications arising from chemotherapy, or a diet supplement for cancer patients (e.g. indole-3-carbinol).

In one aspect, the present disclosure provides an isolated polynucleotide encoding the antibody or an antigen-binding fragment thereof provided herein.

In one aspect, the present disclosure provides a vector comprising the isolated polynucleotide provided herein.

In one aspect, the present disclosure provides a host cell comprising the vector provided herein.

In one aspect, the present disclosure provides a method of expressing the antibody or antigen-binding fragment thereof provided herein, comprising culturing the host cell provided herein under the condition at which the vector provided herein is expressed.

In one aspect, the present disclosure provides a method of treating, reducing the severity of and/or slowing the progression of a VEGFR2-related disease or condition in a subject, comprising administering to the subject a therapeutically effective amount of the antibody or antigen-binding fragment thereof provided herein, or the pharmaceutical composition provided herein.

In certain embodiments, the VEGFR2-related disease or condition is a tumor or an angiogenic disease.

In certain embodiments, the tumor produces VEGF (e.g., VEGF-A) and/or is sensitive to VEGF (e.g., VEGF-A) present in its microenvironment.

In certain embodiments, the tumor is a solid tumor or non-solid tumor.

In certain embodiments, the solid tumor is selected from the group consisting of breast carcinoma, lung carcinoma, colorectal carcinoma, pancreatic carcinoma, glioma and lymphoma, head and neck tumors, neuroendocrine tumors, colorectal tumors, prostate tumors, breast tumors, lung tumors, such as small cell and non-small cell lung tumors, pancreatic tumors, thyroid tumors, ovarian tumors, cervical tumors, kidney tumors, brain tumors, liver tumors, Kaposi's sarcoma, CNS neoplasms, neuroblastomas, capillary hemangioblastomas, meningiomas, cerebral metastases, melanoma, gastrointestinal and renal carcinomas and sarcomas (e.g., gastric cancer), rhabdomyosarcoma, glioblastoma, preferably glioblastoma multiforme, leiomyosarcoma, squamous cell carcinoma, basal cell carcinoma and skin cancers that can be treated by suppressing the growth of malignant keratinocytes, such as human malignant keratinocytes.

In certain embodiments, the tumor is selected from the group consisting of gastric cancer, non-small cell lung cancer, such as large cell lung cancer.

In certain embodiments, the non-solid tumor is selected from the group consisting of leukemia, multiple myeloma and lymphoma, for example, acute myelogenous leukemia (AML), chronic myelogenous leukemia (CML), acute lymphocytic leukemia (ALL), chronic lymphocytic leukemia (CLL), erythrocytic leukemia ormonocytic leukemia, Hodgkin's and non-Hodgkin's lymphoma.

In certain embodiments, the angiogenic disease is selected from the group consisting of atherosclerosis, rheumatoid arthritis (RA), neovascular glaucoma, proliferative retinopathy including proliferative diabetic retinopathy, macular degeneration, hemangiomas, angiofibromas, psoriasis, retinopathy of prematurity (e.g., retrolental fibroplastic), corneal graft rejection, insulin-dependent diabetes mellitus, multiple sclerosis, myasthenia gravis, Chron's disease, autoimmune nephritis, primary biliary cirrhosis, acute pancreatitis, allograph rejection, allergic inflammation, contact dermatitis and delayed hypersensitivity reactions, inflammatory bowel disease, septic shock, osteoporosis, osteoarthritis, cognition defects induced by neuronal inflammation, Osler-Weber syndrome, restinosis, and fungal, parasitic and viral infections, such as cytomegaloviral infections.

In certain embodiments, the subject is human.

In certain embodiments, the administration is via oral, nasal, intravenous, subcutaneous, sublingual, intratumoral, or intramuscular administration.

In certain embodiments, the method provided herein further comprises administering a therapeutically effective amount of a second therapeutic agent.

In certain embodiments, the second therapeutic agent comprises an anti-cancer therapy, optionally the anti-cancer therapy is selected from a chemotherapeutic agent, radiation therapy, an immunotherapy agent, anti-angiogenesis agent (e.g. antagonist of a VEGFR such as VEGFR-1, VEGFR-2, and VEGFR-3), an EGFR antagonist, an PDGFR antagonist, an IGFR antagonist, an NGFR antagonist, an FGFR antagonist, a targeted therapy agent, a cellular therapy agent, a gene therapy agent, a hormonal therapy agent, cytokines, palliative care, surgery for the treatment of cancer (e.g., tumorectomy), one or more anti-emetics, treatments for complications arising from chemotherapy, or a diet supplement for cancer patients (e.g. indole-3-carbinol).

In one aspect, the present disclosure provides a kit comprising an antibody or an antigen-binding fragment thereof provided herein.

In one aspect, the present disclosure provides a method of detecting presence or amount of VEGFR2 in a sample, comprising contacting the sample with the antibody or antigen-binding fragment thereof provided herein, and determining the presence or the amount of VEGFR2 in the sample.

In one aspect, the present disclosure provides use of the antibody or antigen-binding fragment thereof provided herein in the manufacture of a medicament for treating, reducing the severity of and/or slowing the progression of a VEGFR2-related disease or condition in a subject.

In certain embodiments, the VEGFR2-related disease or condition is a tumor or an angiogenic disease.

In certain embodiments, the tumor produces VEGF (e.g., VEGF-A) and/or is sensitive to VEGF (e.g., VEGF-A) present in its microenvironment.

In certain embodiments, the tumor is a solid tumor or non-solid tumor.

In certain embodiments, the angiogenic disease is selected from the group consisting of atherosclerosis, rheumatoid arthritis (RA), neovascular glaucoma, proliferative retinopathy including proliferative diabetic retinopathy, macular degeneration, hemangiomas, angiofibromas, psoriasis, retinopathy of prematurity (e.g., retrolental fibroplastic), corneal graft rejection, insulin-dependent diabetes mellitus, multiple sclerosis, myasthenia gravis, Chron's disease, autoimmune nephritis, primary biliary cirrhosis, acute pancreatitis, allograph rejection, allergic inflammation, contact dermatitis and delayed hypersensitivity reactions, inflammatory bowel disease, septic shock, osteoporosis, osteoarthritis, cognition defects induced by neuronal inflammation, Osler-Weber syndrome, restinosis, and fungal, parasitic and viral infections, such as cytomegaloviral infections.

### BRIEF DESCRIPTION OF FIGURES

FIG. 1A and FIG. 1B show the dose-dependent binding of purified hybridoma antibodies #002, #003, #006, #018, #042, #048, and #054 to human VEGFR2 (hVEGFR2).

FIG. 2 shows the dose-dependent blocking activities of purified hybridoma antibodies for human VEGF-A (hVEGF-A) binding to hVEGFR2.

FIG. 3 shows the binding of hybridoma antibodies to HUVEC cells.

FIG. 4A and FIG. 4B show chimeric antibodies that bind to hVEGFR2 and rhesus VEGFR2.

FIG. 5 shows that the chimeric antibodies blocked the VEGF-A/VEGFR2 interaction.

FIG. 6 shows the binding of chimeric antibodies to HUVEC cells.

FIG. 7A and FIG. 7B show epitope binning using Biotin-mAB002 by competition assay.

FIG. 8A, FIG. 8B and FIG. 8C show the dose-dependent competition assay results.

FIG. 9A, FIG. 9B and FIG. 9C show that the chimeric antibodies inhibit phosphorylation of VEGFR2 induced by VEGF-A.

FIG. 10A and FIG. 10B show humanized antibodies produced in 293T cells binding to human VEGFR2-his by ELISA.

FIG. 11A and FIG. 11B show humanized antibodies produced in CHO cells binding to human VEGFR2-his and rhesus by ELISA.

FIG. 12 shows humanized antibodies that bind specifically to VEGFR2.

FIG. 13A and FIG. 13B show humanized antibodies blocking the binding of VEGF-A to human VEGFR2 or rhesus VEGFR2.

FIG. 14A, FIG. 14B and FIG. 14C show humanized antibodies blocking VEGF-A, C or D binding to VEGFR2.

FIG. 15 shows humanized antibodies binding to HUVEC cells.

FIG. 16 shows that humanized antibody 054 inhibits VEGF-A induced VEGFR2 phosphorylation.

FIG. 17 shows humanized antibodies blocking VEGF-A mediated cell proliferation.

FIG. 18 shows the survival curve of HL-60 engraftment on NOD/SCID mice treated with a benchmark antibody 1121B, chimeric 054 (54-C), chimeric 002 (2-C) or saline.

FIG. 19 shows ELISA binding of Chimeric-DC101 to mouse VEGFR2.

FIG. 20 shows blockade of mouse VEGFR2 binding to human VEGF165 (a splice variant of VEGF-A) by Chimeric-DC101.

FIG. 21 shows tumor growth curve of MKN45 xenograft tumor on nude mice (mean±S.E.M., n=10).

FIG. 22. shows tumor growth curve of H460 xenograft tumor on nude mice (mean±S.E.M., n=10)

FIG. 23 shows tumor growth curve of H1975 xenograft tumor on nude mice (mean±S.E.M., n=10)

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

As used herein, the term “a,” “an,” “the” and similar terms used in the context of the present invention (especially in the context of the claims) are to be construed to cover both the singular and plural unless otherwise indicated herein or clearly contradicted by the context.

The term “antibody” as used herein includes any immunoglobulin, monoclonal antibody, polyclonal antibody, multivalent antibody, bivalent antibody, monovalent antibody, multispecific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (V_{H}) and a first, second, and third constant region (C_{H1}, C_{H2}, C_{H3}, respectively); mammalian light chains are classified as X or x, while each light chain consists of a variable region (V_{L}) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding domains disclosed herein may be defined or identified by the conventions of Kabat, IMGT, AbM, Chothia, or Al-Lazikani (Al-Lazikani, B., Chothia, C., Lesk, A. M., J. Mol. Biol., 273(4), 927 (1997); Chothia, C. et al., J Mol Biol. Dec. 5; 186(3):651-63 (1985); Chothia, C. and Lesk, A. M., J.Mol.Biol., 196,901 (1987); N. R. Whitelegg et al, Protein Engineering, v13(12), 819-824 (2000); Chothia, C. et al., Nature. Dec. 21-28; 342(6252):877-83 (1989); Kabat E.A. et al., National Institutes of Health, Bethesda, Md. (1991); Marie-Paule Lefranc et al, Developmental and Comparative Immunology, 27: 55-77 (2003); Marie-Paule Lefranc et al, Immunome Research, 1(3), (2005); Marie-Paule Lefranc, Molecular Biology of B cells (second edition), chapter 26, 481-514, (2015)). The three CDRs are interposed between flanking stretches known as framework regions (FRs), which are more highly conserved than the CDRs and form a scaffold to support the hypervariable loops. The constant regions of the heavy and light chains are not involved in antigen-binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain). In certain embodiments, the antibody provided herein encompasses any antigen-binding fragments thereof.

As used herein, the term “antigen-binding fragment” refers to an antibody fragment formed from a fragment of an antibody comprising one or more CDRs, or any other antibody portion that binds to an antigen but does not comprise an intact native antibody structure. Examples of antigen-binding fragment include, without limitation, a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody. An antigen-binding fragment is capable of binding to the same antigen to which the parent antibody binds. In certain embodiments, an antigen-binding fragment may comprise one or more CDRs from a particular human antibody.

“Fab” with regard to an antibody refers to a monovalent antigen-binding fragment of the antibody consisting of a single light chain (both variable and constant regions) bound to the variable region and first constant region of a single heavy chain by a disulfide bond. Fab can be obtained by papain digestion of an antibody at the residues proximal to the N-terminus of the disulfide bond between the heavy chains of the hinge region.

“Fab” refers to a Fab fragment that includes a portion of the hinge region, which can be obtained by pepsin digestion of an antibody at the residues proximal to the C-terminus of the disulfide bond between the heavy chains of the hinge region and thus is different from Fab in a small number of residues (including one or more cysteines) in the hinge region.

“F(ab′)2” refers to a dimer of Fab′ that comprises two light chains and part of two heavy chains.

“Fc” with regard to an antibody refers to that portion of the antibody consisting of the second and third constant regions of a first heavy chain bound to the second and third constant regions of a second heavy chain via disulfide bond. IgG and IgM Fc regions contain three heavy chain constant regions (second, third and fourth heavy chain constant regions in each chain). It can be obtained by papain digestion of an antibody. The Fc portion of the antibody is responsible for various effector functions such as ADCC, ADCP and CDC, but does not function in antigen binding.

“Fv” with regard to an antibody refers to the smallest fragment of the antibody to bear the complete antigen binding site. A Fv fragment consists of the variable region of a single light chain bound to the variable region of a single heavy chain. A “dsFv” refers to a disulfide-stabilized Fv fragment that the linkage between the variable region of a single light chain and the variable region of a single heavy chain is a disulfide bond.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)). A “scFv dimer” refers to a single chain comprising two heavy chain variable regions and two light chain variable regions with a linker. In certain embodiments, an “scFv dimer” is a bivalent diabody or bivalent ScFv (BsFv) comprising V_{H}-V_{L} (linked by a peptide linker) dimerized with another V_{H}-V_{L}moiety such that V_{H}'s of one moiety coordinate with the V_{L}'s of the other moiety and form two binding sites which can target the same antigens (or eptipoes) or different antigens (or eptipoes). In other embodiments, a “scFv dimer” is a bispecific diabody comprising V_{H1}—V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

“Single-chain Fv-Fc antibody” or “scFv-Fc” refers to an engineered antibody consisting of a scFv connected to the Fc region of an antibody.

“Camelized single domain antibody,” “heavy chain antibody,” “nanobody” or “HCAb” refers to an antibody that contains two V_{H} domains and no light chains (Riechmann L. and Muyldermans S., J Immunol Methods. Dec. 10; 231(1-2):25-38 (1999); Muyldermans S., J Biotechnol. Jun.; 74(4):277-302 (2001); WO94/04678; WO94/25591; U.S. Pat. No. 6,005,079). Heavy chain antibodies were originally obtained from Camelidae (camels, dromedaries, and llamas). Although devoid of light chains, camelized antibodies have an authentic antigen-binding repertoire (Hamers-Casterman C. et al., Nature. Jun. 3; 363(6428):446-8 (1993); Nguyen VK. et al. “Heavy-chain antibodies in Camelidae; a case of evolutionary innovation,” Immunogenetics. Apr.; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. Nov.; 21(13):3490-8. Epub 2007 Jun. 15 (2007)). “Diabodies” include small antibody fragments with two antigen-binding sites, wherein the fragments comprise a V_{H} domain connected to a V_{L} domain in a single polypeptide chain (V_{H}-V_{L} or V_{L}-V_{H}) (see, e.g., Holliger P. et al., Proc Natl Acad Sci USA. Jul. 15; 90(14):6444-8 (1993); EP404097; WO93/11161). The two domains on the same chain cannot be paired, because the linker is too short, thus, the domains are forced to pair with the complementary domains of another chain, thereby creating two antigen-binding sites. The antigen-binding sites may target the same of different antigens (or epitopes).

A “domain antibody” refers to an antibody fragment containing only the variable region of a heavy chain or the variable region of a light chain. In certain embodiments, two or more V_{H}domains are covalently joined with a peptide linker to form a bivalent or multivalent domain antibody. The two V_{H} domains of a bivalent domain antibody may target the same or different antigens.

In certain embodiments, a “(dsFv)₂” comprises three peptide chains: two V_{H} moieties linked by a peptide linker and bound by disulfide bridges to two V_{L} moieties.

In certain embodiments, a “bispecific ds diabody” comprises V_{H1}—V_{L2} (linked by a peptide linker) bound to V_{L1}-V_{H2} (also linked by a peptide linker) via a disulfide bridge between V_{H1} and V_{L}1.

In certain embodiments, a “bispecific dsFv” or “dsFv-dsFv” comprises three peptide chains: a V_{H}1-V_{H2} moiety wherein the heavy chains are bound by a peptide linker (e.g., a long flexible linker) and paired via disulfide bridges to V_{L1} and V_{L2} moieties, respectively. Each disulfide paired heavy and light chain has a different antigen specificity.

The term “humanized” as used herein means that the antibody or antigen-binding fragment comprises CDRs derived from non-human animals, FR regions derived from human, and when applicable, constant regions derived from human. In certain embodiments, the amino acid residues of the variable region framework of the humanized hVEGFR2 antibody are substituted for sequence optimization. In certain embodiments, the variable region framework sequences of the humanized hVEGFR2 antibody chain are at least 65%, 70%, 75%, 80%, 85%, 90%, 95% or 100% identical to the corresponding human variable region framework sequences.

The term “chimeric” as used herein refers to an antibody or antigen-binding fragment that has a portion of heavy and/or light chain derived from one species, and the rest of the heavy and/or light chain derived from a different species. In an illustrative example, a chimeric antibody may comprise a constant region derived from human and a variable region derived from a non-human species, such as from mouse.

The term “germline sequence” refers to the nucleic acid sequence encoding a variable region amino acid sequence or subsequence that shares the highest determined amino acid sequence identity with a reference variable region amino acid sequence or subsequence in comparison to all other known variable region amino acid sequences encoded by germline immunoglobulin variable region sequences. The germline sequence can also refer to the variable region amino acid sequence or subsequence with the highest amino acid sequence identity with a reference variable region amino acid sequence or subsequence in comparison to all other evaluated variable region amino acid sequences. The germline sequence can be framework regions only, complementarity determining regions only, framework and complementarity determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The germline nucleic acid or amino acid sequence can have at least about 90%, 91, 92%, 93%, 94%, 95%, 96%, 97%, 98%, 99%, or 100% sequence identity with the reference variable region nucleic acid or amino acid sequence. Germline sequences can be determined, for example, through the publicly available international ImMunoGeneTics database (IMGT) and V-base.

“Anti-hVEGFR2 antibody” or “an antibody against hVEGFR2” as used herein refers to an antibody that is capable of specific binding to human VEGFR2 with a sufficient affinity, for example, to provide for diagnostic and/or therapeutic use.

The term “affinity” as used herein refers to the strength of non-covalent interaction between an immunoglobulin molecule (i.e. antibody) or fragment thereof and an antigen.

The term “specific binding” or “specifically binds” as used herein refers to a non-random binding reaction between two molecules, such as for example between an antibody and an antigen.

In certain embodiments, the antibodies or antigen-binding fragments provided herein specifically bind to hVEGFR2 with a binding affinity (K_{D}) of ≤10⁻⁶ M (e.g., ≤5×10⁻⁷ M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤4×10⁻⁹ M, ≤3×10⁻⁹ M,≤2×10⁻⁹ M, or ≤10⁻⁹ M. K_{D} used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry (such as FACS) method. In certain embodiments, the K_{D} value can be appropriately determined by using flow cytometry method. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically a specific or selective binding reaction will produce a signal at least twice over the background signal and more typically at least 10 to 100 times over the background.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum correspondence. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S.F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M.A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm. In certain embodiments, the non-identical residue positions may differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference.

As used herein, a “homologue sequence” and “homologous sequence” are used interchangeable and refer to polynucleotide sequences (or its complementary strand) or amino acid sequences that have sequences identity of at least 80% (e.g. at least 85%, 88%, 90%, 91%, 92%, 93%, 94%, 95%, 96%, 97%, 98%, 99%) to another sequences when optionally aligned.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An isolated “nucleic acid” or “polynucleotide” are used interchangeably and refer to the sequence of an isolated nucleic acid molecule. In certain embodiments, an “isolated antibody or antigen-binding fragment thereof” refers to the antibody or antigen-binding fragments having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The ability to “block(s) binding” as used herein refers to the ability of an antibody or antigen-binding fragment to inhibit the binding interaction between two molecules (e.g. VEGR-A and hVEGFR2) to any detectable degree. In certain embodiments, an antibody or antigen-binding fragment that blocks binding between two molecules (e.g. VEGR-A and hVEGFR2) inhibits the binding interaction between the two molecules by at least 50%. In certain embodiments, this inhibition may be greater than 60%, greater than 70%, greater than 80%, or greater than 90%.

The term “antibody drug conjugate” as used herein refers to the linkage of an antibody or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed in order to form the antibody drug conjugate. Additionally, the antibody drug conjugate can be provided in the form of a fusion protein that may be expressed from a polynucleotide encoding the conjugate. As used herein, “fusion protein” refers to proteins created through the joining of two or more genes or gene fragments which originally coded for separate proteins (including peptides and polypeptides). Translation of the fusion gene results in a single protein with functional properties derived from each of the original proteins.

The term “subject” includes human and non-human animals. Non-human animals include all vertebrates, e.g., mammals and non-mammals, such as non-human primates, mouse, rat, cat, rabbit, sheep, dog, cow, chickens, amphibians, and reptiles. Except when noted, the terms “patient” or “subject” are used herein interchangeably.

“Effector functions” or “antibody effector functions” as used herein refer to biological activities attributable to the binding of Fc region of an antibody to its effectors such as C1 complex and Fc receptor. Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and antibody dependent cell mediated phagocytosis (ADCP), where nonspecific cytotoxic cells that express FcγRs recognize bound antibody on a target cell and subsequently cause phagocytosis of the target cell. Effector functions include both those that operate after the binding of an antigen and those that operate independent of antigen binding.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “vector” as used herein refers to a vehicle into which a genetic element may be operably inserted so as to bring about the expression of that genetic element, such as to produce the protein, RNA or DNA encoded by the genetic element, or to replicate the genetic element. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g. expression vectors) containing the nucleic acid sequence provided herein encoding the antibody or antigen-binding fragment thereof, at least one promoter (e.g. SV40, CMV, EF-1α) operably linked to the nucleic acid sequence, and at least one selection marker.

The “host cell” as used herein refers to a cell into which an exogenous polynucleotide and/or a vector has been introduced.

The term “VEGFR2”, used interchangeably with the terms “VEGF receptor 2”, “kinase insert domain-containing receptor (KDR)”, “CD309”, or “fetal liver kinase 1 (FLK1)” is one type of VEGF receptors (VEGFRs), which is a type III receptor tyrosine kinase characterized by having, typically, 5 or 7 immunoglobulin-like loops in their amino-terminal extracellular receptor ligand-binding domains, as well as a transmembrane region and a carboxy-terminal intracellular catalytic domain interrupted by an insertion of hydrophilic interkinase sequences of variable lengths called the kinase insert domain (Kaipainen et al., J. Exp. Med., 178:2077-88 (1993); Terman et al., Oncogene, 6:1677-83 (1991)). Other types of VEGFRs include fins-like tyrosine kinase receptor (fit-1) or VEGFR1, and VEGFR3 (fit-4) (Shibuya et al., Oncogene, 5:519-24 (1990)). The amino-terminal extracellular receptor ligand-binding domains are responsible for the affinity between the ligand (e.g., vascular endothelial growth factor A (VEGF-A)) and the domains. The carboxy-terminal intracellular catalytic domain is responsible for the initiation of the signaling pathway involved in, for example, cell growth. Expression of VEGFRs can be found on endothelial cells during, e.g., embryogenesis and tumor formation (Millauer, B. et al. Cell, 72:835-46 (1993); Plate, K. et al. (1993)). Expression of VEGFRs can also be found on non-endothelial cells, such as tumor cells, especially tumor cells producing VEGF, e.g., leukemic cells, (Fielder et al., Blood 89:1870-5 (1997) and Bellamy et al., Cancer Res. 59728-33 (1999).).

Human VEGFR2 (hVEGFR2) used herein comprises an amino acid sequence that can be
accessed via the accession number AAI31823.1 in the NCBI database or an
amino acid sequence of SEQ ID NO: 39
(MQSKVLLAVALWLCVETRAASVGLPSVSLDLPRLSIQKDILTIKANTTLQITCRGQRDLDWLWPNNQ
SGSEQRVEVTECSDGLFCKTLTIPKVIGNDTGAYKCFYRETDLASVIYVYVQDYRSPFIASVSDQHG
VVYITENKNKTVVIPCLGSISNLNVSLCARYPEKRFVPDGNRISWDSKKGFTIPSYMISYAGMVFCE
AKINDESYQSIMYIVVVVGYRIYDVVLSPSHGIELSVGEKLVLNCTARTELNVGIDENWEYPSSKHQ
HKKLVNRDLKTQSGSEMKKELSTLTIDGITRSDQGLYTCAASSGLMTKKNSTFVRVHEKPFVAFGSG
MESLVEATVGERVRIPAKYLGYPPPEIKWYKNGIPLESNHTIKAGHVLTIMEVSERDTGNYTVILTN
PISKEKQSHVVSLVVYVPPQIGEKSLISPVDSYQYGTTQTLTCTVYAIPPPHHIHWYWQLEEECANE
PSHAVSVTNPYPCEEWRSVEDFQGGNKIEVNKNQFALIEGKNKTVSTLVIQAANVSALYKCEAVNKV
GRGERVISFHVTRGPEITLQPDMQPTEQESVSLWCTADRSTFENLTWYKLGPQPLPIHVGELPTPVC
KNLDTLWKLNATMESNSTNDILIMELKNASLQDQGDYVCLAQDRKTKKRHCVVRQLTVLERVAPTIT
GNLENQTTSIGESIEVSCTASGNPPPQIMWFKDNETLVEDSGIVLKDGNRNLTIRRVRKEDEGLYTC
QACSVLGCAKVEAFFIIEGAQEKTNLEIIILVGTAVIAMFFWLLLVIILRTVKRANGGELKTGYLSI
VMDPDELPLDEHCERLPYDASKWEFPRDRLKLGKPLGRGAFGQVIEADAFGIDKTATCRTVAVKMLK
EGATHSEHRALMSELKILIHIGHHLNVVNLLGACTKPGGPLMVIVEFCKFGNLSTYLRSKRNEFVPY
KTKGARFRQGKDYVGAIPVDLKRRLDSITSSQSSASSGFVEEKSLSDVEEEEAPEDLYKDELTLEHL
ICYSFQVAKGMEFLASRKCIHRDLAARNILLSEKNVVKICDFGLARDIYKDPDYVRKGDARLPLKWM
APETIFDRVYTIQSDVWSFGVLLWEIFSLGASPYPGVKIDEEFCRRLKEGTRMRAPDYTTPEMYQTM
LDCWHGEPSQRPTFSELVEHLGNLLQANAQQDGKDYIVLPISETLSMEEDSGLSLPTSPVSCMEEEE
VCDPKFHYDNTAGISQYLQNSKRKSRPVSVKTFEDIPLEEPEVKVIPDDNQTDSGMVLASEELKTLE
DRTKLSPSFGGMVPSKSRESVASEGSNQTSGYQSGYHSDDTDTTVYSSEEAELLKLIEIGVQTGSTA
QILQPDSGTTLSSPPV).
Mouse VEGFR2 used herein comprises an amino acid sequence that can be accessed
via the accession number P35918.1 in the NCBI database or an amino acid sequence
of SEQ ID NO: 100
(MESKALLAVALWFCVETRAASVGLPGDFLHPPKLSTQKDILTILANTTLQITCRGQRDLDWLWPNAQ
RDSEERVLVTECGGGDSIFCKTLTIPRVVGNDTGAYKCSYRDVDIASTVYVYVRDYRSPFIASVSDQ
HGIVYITENKNKTVVIPCRGSISNLNVSLCARYPEKRFVPDGNRISWDSEIGFTLPSYMISYAGMVE
CEAKINDETYQSIMYIVVVVGYRIYDVILSPPHEIELSAGEKLVLNCTARTELNVGLDFTWHSPPSK
SHHKKIVNRDVKPFPGTVAKMFLSTLTIESVTKSDQGEYTCVASSGRMIKRNRTFVRVHTKPFIAFG
SGMKSLVEATVGSQVRIPVKYLSYPAPDIKWYRNGRPIESNYTMIVGDELTIMEVTERDAGNYTVIL
TNPISMEKQSHMVSLVVNVPPQIGEKALISPMDSYQYGTMQTLTCTVYANPPLHHIQWYWQLEEACS
YRPGQTSPYACKEWRHVEDFQGGNKIEVTKNQYALIEGKNKTVSTLVIQAANVSALYKCEAINKAGR
GERVISFHVIRGPEITVQPAAQPTEQESVSLLCTADRNTFENLTWYKLGSQATSVHMGESLTPVCKN
LDALWKLNGTMFSNSINDILIVAFQNASLQDQGDYVCSAQDKKTKKRHCLVKQLIILERMAPMITGN
LENQTTTIGETIEVTCPASGNPTPHITWFKDNETLVEDSGIVLRDGNRNLTIRRVRKEDGGLYTCQA
CNVLGCARAETLFIIEGAQEKINLEVIILVGTAVIAMFFWLLLVILVRTVKRANEGELKTGYLSIVM
DPDELPLDERCERLPYDASKWEFPRDRLKLGKPLGRGAFGQVIEADAFGIDKTATCKTVAVKMLKEG
ATHSEHRALMSELKILIHIGHHLNVVNLLGACTKPGGPLMVIVEFSKFGNLSTYLRGKRNEFVPYKS
KGARFRQGKDYVGELSVDLKRRLDSITSSQSSASSGFVEEKSLSDVEEEEASEELYKDELTLEHLIC
YSFQVAKGMEFLASRKCIHRDLAARNILLSEKNVVKICDFGLARDIYKDPDYVRKGDARLPLKWMAP
ETIFDRVYTIQSDVWSFGVLLWEIFSLGASPYPGVKIDEEFCRRLKEGTRMRAPDYTTPEMYQTMLD
CWHEDPNQRPSFSELVEHLGNLLQANAQQDGKDYIVLPMSETLSMEEDSGLSLPTSPVSCMEEEEVC
DPKFHYDNTAGISHYLQNSKRKSRPVSVKTFEDIPLEEPEVKVIPDDSQTDSGMVLASEELKTLEDR
NKLSPSFGGMMPSKSRESVASEGSNQTSGYQSGYHSDDTDTTVYSSDEAGLLKMVDAAVHADSGTTL
QLTSCLNGSGPVPAPPPTPGNHERGAA).
Rhesus VEGFR2 used herein comprises an amino acid sequence that can be accessed
via the accession number XP_014994176.1 in the NCBI database or an amino
acid sequence of SEQ ID NO: 101
(MASKVLLAVALWLCVETRAASVGLPSVSLDLPRLSIQKDILTIKANTTLQITCRGQRDLDWLWPNNQ
SGSEQRVEVTECSDGLFCKTLTIPKVIGNDTGAYKCFYRETDLASVIYVYVQDYRSPFIASVSDQHG
VVYITENKNKTVVIPCLGSISNLNVSLCARYPEKRFVPDGNRISWDSKKGFTIPSYMISYAGMVFCE
AKINDESYQSIMYIVVVVGYRIYDVVLSPSHGVELSVGEKLVLNCTARTELNVGIDENWEYPSSKHQ
HKKLVNRDLKTQSGSEMKKFLSTLTIDGVTRSDQGLYTCAASSGLMTKKNSTFVRVHEKPFVAFGSG
MESLVEATVGERVRIPVKYLGYPPPEIKWYKNGIPLESNHTVKVGHVLTIMEVSERDTGNYTVILTN
PISKEKQSHVVSLVVYVPPQIGEKSLISPVDSYQYGTTQTLTCTVYAIPPPHHIHWYWQLEEECPNE
PSQAVSVTNPYPCEEWRSVEDFQGGNKIEVNKNQFALIEGKNKTVSTLVIQAANVSALYKCEAVNKV
GRGERVISFHVTRGPEITLQPDLQPTEQESVSLWCTADKSTFENLTWYKLGPQPLPVHVGELPTPVC
KNLDTLWKLNATIFSNSINDILIMELKNASLQDQGDYVCVAQDRKTKKRHCVVRQLTVLERVAPMIT
GNLENQTTSIGETIEVSCTASGNPPPQIMWEKDNETLVEDSGIVLKDGNRNLTIRRVRKEDEGLYTC
QACSVLGCAKVEAFFIIEGAQEKTNLEIIILVGTAVIAMFFWLLLVIILRTVKRANGGELKTGYLSI
VMDPDELPLDEHCERLPYDASKWEFPRDRLKLGKPLGRGAFGQVIEADAFGIDKTATCRTVAVKMLK
EGATHSEHRALMSELKILIHIGHHLNVVNLLGACTKPGGPLMVIVEFCKFGNLSTYLRSKRNEFVPY
KTKGARFRQGKDYVGAIPVDLKRRLDSITSSQSSASSGFVEEKSLSDVEEEEAPEDLYKDELTLEHL
ICYSFQVAKGMEFLASRKCIHRDLAARNILLSEKNVVKICDFGLARDIYKDPDYVRKGDARLPLKWM
APETIFDRVYTIQSDVWSFGVLLWEIFSLGASPYPGVKIDEEFCRRLKEGTRMRAPDYTTPEMYQTM
LDCWHGEPSQRPTFSELVEHLGNLLQANAQQDGKDYIVLPISETLSMEEDSGLSLPTSPVSCMEEEE
VCDPKFHYDNTAGISQYLQNSKRKSRPVSVKTFEDIPLEEPEVKVIPDDNQTDSGMVLASEELKTLE
DRTKLAPSFSGMVSSKSRESVASEGSNQTSGYQSGYHSDDTDTTVYSSEEAELLKLIEIGVQTGSTA
QILQPDSGTTLSSPPV)

A “hVEGFR2-related” disease or condition as used herein refers to any disease or condition caused by, exacerbated by, or otherwise linked to increased or decreased expression or activities of hVEGFR2. In some embodiments, the hVEGFR2 related condition is, for example, cancer, or an angiogenic disease.

“VEGF-A”, used interchangeably with the term “vascular endothelial growth factor A” refers to a highly conserved dimeric glycoprotein or transcript splice variants thereof, such as VEGFA206, VEGFA189, VEGFA165 and VEGFA121 containing 206, 189, 165 and 121 amino acids respectively. VEGF-A is a member of the VEGF family, which also encompasses other members, such as VEGF-B, VEGF-C, and VEGF-D.

“Cancer” as used herein refers to any medical condition characterized by malignant cell growth or neoplasm, abnormal proliferation, infiltration or metastasis, and includes both solid tumors and non-solid cancers (e.g. hematologic malignancies) such as leukemia. As used herein “solid tumor” refers to a solid mass of neoplastic and/or malignant cells.

“Angiogenic disease” as used herein refers to any medical condition associated with abnormalities during angiogenesis, a process of growing new blood vessels. Such abnormalities include, without limitation, excessive angiogenesis, insufficient angiogenesis, improper angiogenesis and damaging angiogenesis. Such medical conditions characterized by e.g., excessive or damaging angiogenesis include, without limitation, cancer, diabetic retinopathies, age-related macular degeneration and atherosclerosis, stroke and myocardial infarction, rheumatoid arthritis.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

Reference to “about” a value or parameter herein includes (and describes) embodiments that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g. within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

### Anti-hVEGFR2 Antibodies

The present disclosure provides anti-hVEGFR2 antibodies and antigen-binding fragments thereof. The anti-hVEGFR2 antibodies and antigen-binding fragments provided herein are capable of specifically binding to hVEGFR2 or hVEGFR2-expressing cells. “Specifically binding” as used herein means a binding affinity (e.g. K_{D}) of ≤10⁻⁶ M (e.g., ≤5×10⁻⁷ M, ≤2×10⁻⁷ M, ≤10⁻⁷ M, ≤5×10⁻⁸ M, ≤2×10⁻⁷ M, ≤10⁻⁸ M, 5×10⁻⁹ M, ≤4×10⁻⁹ M, ≤3×10⁻⁹ M,≤2×10⁻⁹ M, or ≤10⁻⁹ M).

i. Binding Affinity

Binding affinity of the anti-hVEGFR2 antibodies and antigen-binding fragments provided herein can be represented by K_{D} value, which represents the ratio of dissociation rate to association rate (k_{off}/kₒₙ) when the binding between the antigen and antigen-binding molecule reaches equilibrium. Low-affinity antibodies generally bind antigen slowly and tend to dissociate readily, whereas high-affinity antibodies generally bind antigen faster and tend to remain bound longer. The antigen-binding affinity (e.g. K_{D}) can be appropriately determined using any suitable methods known in the art, including, for example, Kinetic Exclusion Assay (KinExA), Biacore, Fortebio or flow cytometry.

In certain embodiments, the “K_{D}” or “K_{D} value” according to the present disclosure is in an embodiment measured by Biacore assay, performed with the anti-hVEGFR2 antibody and hVEGFR2 as described by the following assay that measures solution binding affinity of an anti-hVEGFR2 antibody. In general, the Biacore works by equilibrating a constant amount of one binding partner (CBP) with a varying concentration of the other binding partner (Titrant), and then capture a portion of the free CBP by fluorescence labeled secondary antibody in a short contact time which is less than the time needed for dissociation of the pre-formed CBP-Titrant complex. The fluorescence signals generated from the captured CBP are directly proportional to the concentration of free CBP in the equilibrated samples, and are used to generate a binding curve (percent free CBP vs. total Titrant concentration) when measured in a series. More details are available from Schreiber, G., Fersht, A. R. Nature Structural Biology. 1996, 3(5), 427-431. When anti-hVEGFR2 antibody is used as CBP with a constant amount, then hVEGFR2 protein can be used as the Titrant, or vice versa. In certain embodiments, the K_{D} of the anti-hVEGFR2 antibody or antigen-binding fragments thereof is determined in accordance to the method as described in Example 16 in the present disclosure.

Other methods suitable for measurement of Kd may also be used under applicable circumstances, for example, radio labelled antigen-binding assay (see, e.g. Chen, et al., (1999) J. Mol Biol 293:865-881).

In certain embodiments, the binding affinity of the anti-hVEGFR2 antibody is measured by flow cytometry. In general, hVEGFR2-expressing cells (e.g., HUVECs) are incubated with a range of concentrations of an anti-hVEGFR2 antibody, followed by incubation with a fluorescently labelled secondary antibody, and then analyzed for fluorescent signal intensity. In certain embodiments, the binding affinity of the anti-hVEGFR2 antibody or antigen-binding fragments thereof is determined in accordance to the method as described in Example 6 in the present disclosure.

In certain embodiments, the anti-hVEGFR2 antibodies and the antigen-binding fragments thereof provided herein specifically bind to hVEGFR2 (or a cell expressing hVEGFR2) at a K_{D} value of no more than 6, 5, 4, 3, 2, 1 or 0.5 nM as measured by Biacore assay. In certain embodiments, the anti-hVEGFR2 antibodies and the antigen-binding fragments thereof provided herein specifically bind to hVEGFR2 (or a cell expressing hVEGFR2) at a K_{D} value of no more than 0.089 nM (or no more than 0.001 nM) as measured by Biacore assay.

Alternatively, binding affinity of the anti-hVEGFR2 antibodies and antigen-binding fragments provided herein to hVEGFR2 can also be represented by “half maximal effective concentration” (EC₅₀) value, which refers to the concentration of an antibody where 50% of its maximal effect (e.g., binding) is observed. The EC₅₀ value can be measured by methods known in the art, for example, sandwich assay such as ELISA, Western Blot, flow cytometry assay, and other binding assay. In certain embodiments, the anti-hVEGFR2 antibodies and the fragments thereof provided herein specifically bind to recombinant hVEGFR2 at an EC50 value of no more than 80 ng/ml (or no more than 70, 65, 60, 55, 50, 45, 40, 35, 30, 25, 20, 15, 12, or 10, 9 or 8 ng/ml) as measured by ELISA. In certain embodiments, the anti-hVEGFR2 antibodies and the fragments thereof provided herein specifically bind to recombinant hVEGFR2 at an EC50 value of no more than 35, 24, 15, 10, 9, 8, 7 or 6 ng/ml as measured by ELISA. In certain embodiments, the anti-hVEGFR2 antibodies and the fragments thereof provided herein cross-reactive to mouse VEGFR2 having an EC50 value of no more than 35 ng/ml as measured by ELISA. In certain embodiments, the anti-hVEGFR2 antibodies and the fragments thereof provided herein binds to hVEGFR2 at an EC50 value no more than 60% of that of 1121B, as measured by ELISA. In such embodiment, the anti-hVEGFR2 antibodies and the fragments thereof comprises a HCDR1 comprising the sequence of SEQ ID NO: 31, a HCDR2 comprising the sequence of SEQ ID NO: 32 or SEQ ID NO: 37, a HCDR3 comprising the sequence of SEQ ID NO: 33, a LCDR1 comprising the sequence of SEQ ID NO: 28, a LCDR2 comprising the sequence of SEQ ID NO: 29, and a LCDR3 comprising the sequence of SEQ ID NO: 30. In certain embodiments, the anti-hVEGFR2 antibodies and the fragments thereof provided herein cross-reactive to rhesus VEGFR2 having an EC50 value of no more than 30, 25, 20, 15, 10 or 9 ng/ml as measured by ELISA.

In certain embodiments, the anti-hVEGFR2 antibodies and the fragments thereof provided herein specifically bind to a cell expressing hVEGFR2 (e.g., HUVEC) at an EC50 value of no more than 160 ng/ml (or no more than 150, 140, 130, 120, 110, 100, 90, 80, 70, 65, 55, 50, 45, 40, 35, 30 or 25 ng/ml) as measured by flow cytometry.

In certain embodiments, the antibodies and antigen-binding fragments thereof provided herein exhibits competitive VEGFR2 binding property that effectively blocks binding of VEGFR2 to VEGF-A. The blocking effect of the antibodies and antigen-binding fragments thereof provided herein can be measured using ELISA as described in, for example, Example 5 of the present disclosure. In certain embodiments, the blocking effect of the antibodies and antigen-binding fragments thereof provided herein can be expressed in IC50, which indicates the concentration of the antibodies and antigen-binding fragments thereof provided herein at which the binding of VEGFR2 to VEGF-A is decreased by 50% in presence of the antibodies and antigen-binding fragments thereof of the present disclosure. In certain embodiments, the IC50 of the antibodies and antigen-binding fragments thereof provided herein is in a range from 0.001 μg/ml to 2.5 μg/ml, from 0.005 μg/ml to 0.5 μg/ml, from 0.05 μg/ml to 0.4 μg/ml or from 0.1 μg/ml to 0.2 μg/ml. In certain embodiments, the antibodies and antigen-binding fragments thereof provided herein blocks VEGF-A induced proliferation of hVEGFR2-expressing cells at an IC50 value no more than 82% of that of 1121B, as measured by cell viability assay. In such embodiment, the antibodies and antigen-binding fragments thereof provided herein comprises the HCDR1 comprises the sequence of SEQ ID NO: 31, the HCDR2 comprises the sequence of SEQ ID NO: 37, and the HCDR3 comprises the sequence of SEQ ID NO: 33, the LCDR1 comprises the sequence of SEQ ID NO: 28, the LCDR2 comprises the sequence of SEQ ID NO: 29, and the LCDR3 comprises the sequence of SEQ ID NO: 30, preferably expressed in CHO.

### Epitope

In certain embodiments, the anti-hVEGFR2 antibody or an antigen-binding fragment thereof provided herein binds to an epitope comprising at least one or more (e.g. one, two, three or more) of amino acid residues at positions Y137A, R164A, Y165A, V218A, Y221A, R222A, E251A, L252A, N253A, G255A, D257A, K286A, G312A, L313A, M314A, T315A and K316A of hVEGFR2 having the amino acid sequence of SEQ ID NO: 103.

The term “epitope” as used herein refers to the specific group of atoms or amino acids on an antigen to which an antibody binds. An epitope can include specific amino acids, sugar side chains, phosphoryl or sulfonyl groups that directly contact an antibody. Those skilled in the art will recognize that it is possible to determine, without undue experimentation, if an antibody binds to the same or overlapping or adjacent epitope as the antibody of present disclosure (e.g., hybridoma/chimeric or humanized antibodies 002, 003, 006, 018, 042, 048 and 054 and any of the chimeric and humanized variant thereof provided herein) by ascertaining whether the two competes for binding to a hVEGFR2 antigen polypeptide.

The term “compete for binding” as used herein with respect to two antigen-binding proteins (e.g. antibodies), means that one antigen-binding protein blocks or reduces binding of the other to the antigen (e.g., human/mouse/rhesus VEGFR2), as determined by a competitive binding assay. Competitive binding assays are well known in the art, include, for example, direct or indirect radioimmunoassay (RIA), direct or indirect enzyme immunoassay (EIA), and sandwich competition assay (see, e.g., Stahli et al., 1983, Methods in Enzymology 9:242-253). Typically, such an assay involves the use of purified antigen bound to a solid surface or cells bearing the antigen, an unlabelled test antibody and a labeled reference antibody. Competitive inhibition is measured by determining the amount of label bound to the solid surface or cells in the presence of the test antibody. Usually the test antibody is present in excess. If two antibodies competes for binding to the hVEGFR2, then the two antibodies bind to the same or overlapping epitope, or an adjacent epitope sufficiently proximal to the epitope bound by the other antibody for steric hindrance to occur. Usually, when a competing antibody is present in excess, it will inhibit (e.g., reduce) specific binding of a test antibody to a common antigen by at least 50-55%, 55-60%, 60-65%, 65-70%, 70-75% 75-80%, 80-85%, 85-90% or more.

In certain embodiments, the epitope or the amino acid residue in the epitope bound by an antibody can be determined by mutating specific residues in the antigen, i.e., hVEGFR2. If an antibody binds to a mutant hVEGFR2 having an amino acid residue mutated, for example to alanine, at significantly reduced level relative to its binding to wild-type hVEGFR2, then this would indicate that the mutated residue is directly involved in the binding of the antibody to hVEGFR2 antigen, or is in close proximity to the antibody when it is bound to the antigen. Such a mutated residue is considered to be within the epitope, and the antibody is considered to specifically bind to an epitope comprising the residue. A significantly reduced level in binding as used herein, means that the binding affinity (e.g. EC50, KD, or binding capacity) between the antibody and the mutant hVEGFR2 is reduced by greater than 10%, 20%, 30%, 40%, 50%, 60%, 70%, 80%, 90%, or more, relative to the binding between the antibody and a wild type hVEGFR2. Such a binding measurement can be conducted using any suitable methods known in the art and disclosed herein, for example, without limitation, KinExA assay, ELISA, Biacore, Fortebio and flow cytometry.

In certain embodiments, the anti-hVEGFR2 antibody or an antigen-binding fragment thereof provided herein exhibit reduced binding (e.g. at least 30% reduced, 50% reduced) to a mutant hVEGFR2 as measured by ELISA, in which a residue in a wild-type hVEGFR2 is substituted with alanine, and the residue is selected from the group consisting of: Y137A, R164A, Y165A, V218A, Y221A, R222A, E251A, L252A, N253A, G255A, D257A, K286A, G312A, L313A, M314A, T315A and K316A relative to SEQ ID NO: 103.

In certain embodiments, the anti-hVEGFR2 antibody or an antigen-binding fragment thereof provided herein binds to an epitope on hVEGFR2, wherein the epitope comprises one or more amino acid residue selected from the group consisting of: Y137, R164, Y165, V218, Y221, R222, E251, L252, N253, G255, D257, K286, G312, L313, M314, T315 and K316 of SEQ ID NO: 103.

In certain embodiments, the anti-hVEGFR2 antibody or an antigen-binding fragment thereof provided herein binds to an epitope comprising Y165 and/or L313 but not any of R222, G255, D257 and T315 of SEQ ID NO: 103.

In certain embodiments, the anti-hVEGFR2 antibody or an antigen-binding fragment thereof provided herein binds to an epitope comprising Y137, V218, Y221, R222, E251, L252, N253, G255, D257, K286, L313, M314, T315, K316 or any combination thereof of SEQ ID NO: 103.

In certain embodiments, the anti-hVEGFR2 antibody or an antigen-binding fragment thereof provided herein binds to an epitope comprising R164, Y165, D257 or any combination thereof of SEQ ID NO: 103.

In certain embodiments, the anti-hVEGFR2 antibody or an antigen-binding fragment thereof provided herein binds to an epitope comprising Y165, Y221, R222, E251, G255, D257, G312, L313, M314, T315, K316 or any combination thereof of SEQ ID NO: 103.

In certain embodiments, the anti-hVEGFR2 antibody or an antigen-binding fragment thereof provided herein binds to an epitope comprising Y165 except R222, G255, D257, L313 and T315 of SEQ ID NO: 103.

In certain embodiments, the anti-hVEGFR2 antibody or an antigen-binding fragment thereof provided herein binds to an epitope comprising Y165, Y221, R222, E251, N253, G255, D257, G312, L313, M314, T315, K316 or any combination thereof of SEQ ID NO: 103.

In certain embodiments, the anti-hVEGFR2 antibody or an antigen-binding fragment thereof provided herein binds to an epitope comprising R164, Y165, D257 or any combination thereof of SEQ ID NO: 103.

In certain embodiments, the anti-hVEGFR2 antibody or an antigen-binding fragment thereof provided herein binds to an epitope comprising Y165, Y221, R222, E251, N253, G255, D257, G312, L313, M314, T315, K316 or any combination thereof of SEQ ID NO: 103.

### Antibody Sequences

In another aspect, the present disclosure provides an anti-hVEGFR2 antibody or an antigen-binding fragment thereof, comprising heavy chain HCDR1, HCDR2 and HCDR3 and/or light chain LCDR1, LCDR2 and LCDR3 sequences, wherein
the HCDR1 sequence comprises SSWMN (SEQ ID NO: 1), DYYMS (SEQ ID NO: 19), X1YGMS (SEQ ID NO: 41), X4YWIM (SEQ ID NO: 44), or a homologue sequence of at least 80% sequence identity thereof,
the HCDR2 sequence comprises RIFPGDGDTYYNGKFQV (SEQ ID NO: 2), FIRNKANGYTTEYSASVKG (SEQ ID NO: 20), SISX₂GGSYTYYADSVX₁₉G (SEQ ID NO: 42), DIYPGX₅GSTNYNEKFKS (SEQ ID NO: 45) or a homologue sequence of at least 80% sequence identity thereof,
the HCDR3 sequence comprises FLDTSGRYVDY (SEQ ID NO: 3), FDYYGSTYCFDY (SEQ ID NO: 21), EX₃DGNYDY (SEQ ID NO: 43), DSNPDY (SEQ ID NO: 46), or a homologue sequence of at least 80% sequence identity thereof;
the LCDR1 sequence comprises KASQDVNTAVA (SEQ ID NO: 4), RASQSVSTSSSSFMH (SEQ ID NO: 22), RSSKSLLYKDGKTYLN (SEQ ID NO: 28), RASESVX₆NSGISFMX₇ (SEQ ID NO: 47) or a homologue sequence of at least 80% sequence identity thereof,
the LCDR2 sequence comprises SASYRYI (SEQ ID NO: 5), YASNLES (SEQ ID NO: 23), LMSTRAS (SEQ ID NO: 29), AASX₈QX₉S (SEQ ID NO: 48) or a homologue sequence of at least 80% sequence identity thereof;
the LCDR3 sequence comprises QQHYRAPLT (SEQ ID NO: 6), QHTWEIPLT (SEQ ID NO: 24), QQLVEYPFT (SEQ ID NO: 30), QQSKEVPYT (SEQ ID NO: 49) or a homologue sequence of at least 80% sequence identity thereof;
   wherein X1 is I or M, X₂ is V or I, X₃ is L or M, X₄ is T or S, X₅ is T or S, X₆ is D or E, X₇ is T or H, X₈ is T or Y, X₉ is G or R, and X₁₉ is E or K.

In one aspect, the present disclosure provides an anti-hVEGFR2 antibody or an antigen-binding fragment thereof provided herein, wherein the HCDR1 comprises an amino acid sequence of SEQ ID NO: 41, the HCDR2 comprises an amino acid sequence of SEQ ID NO: 42, the HCDR3 comprises an amino acid sequence of SEQ ID NO: 43, the LCDR1 comprises a sequence of SEQ ID NO: 28, the LCDR2 comprises a sequence of SEQ ID NO: 29, and the LCDR3 comprises a sequence of SEQ ID NO: 30.

In one aspect, the present disclosure provides an anti-hVEGFR2 antibody or an antigen-binding fragment thereof provided herein, wherein
a) the HCDR1 comprises the sequence of SEQ ID NO: 25, a HCDR2 comprises the sequence of SEQ ID NO: 26, the HCDR3 comprises the sequence of SEQ ID NO: 27; the LCDR1 comprises the sequence of SEQ ID NO: 28, the LCDR2 comprises the sequence of SEQ ID NO: 29, and the LCDR3 comprises the sequence of SEQ ID NO: 30; or
b) the HCDR1 comprises the sequence of SEQ ID NO: 31, the HCDR2 comprises the sequence of SEQ ID NO: 32 or SEQ ID NO: 37, and the HCDR3 comprises the sequence of SEQ ID NO: 33, the LCDR1 comprises the sequence of SEQ ID NO: 28, the LCDR2 comprises the sequence of SEQ ID NO: 29, and the LCDR3 comprises the sequence of SEQ ID NO: 30; or
c) the HCDR1 comprises the sequence of SEQ ID NO: 34, the HCDR2 comprises the sequence of SEQ ID NO: 35 or SEQ ID NO: 37, and the HCDR3 comprises the sequence of SEQ ID NO: 36, the LCDR1 comprises the sequence of SEQ ID NO: 28, the LCDR2 comprises the sequence of SEQ ID NO: 29, and the LCDR3 comprises the sequence of SEQ ID NO: 30.

In one aspect, the present disclosure provides an anti-hVEGFR2 antibody or an antigen-binding fragment thereof provided herein, wherein the HCDR1 comprises an amino acid sequence of SEQ ID NO: 44, the HCDR2 comprises an amino acid sequence of SEQ ID NO: 45, the HCDR3 comprises an amino acid sequence of SEQ ID NO: 46, the LCDR1 comprises a sequence of SEQ ID NO: 47, the LCDR2 comprises a sequence of SEQ ID NO: 48, and the LCDR3 comprises a sequence of SEQ ID NO: 49.

In one aspect, the present disclosure provides an anti-hVEGFR2 antibody or an antigen-binding fragment thereof provided herein, wherein
a) the HCDR1 comprises the sequence of SEQ ID NO: 7, a HCDR2 comprises the sequence of SEQ ID NO: 8, the HCDR3 comprises the sequence of SEQ ID NO: 9; the LCDR1 comprises the sequence of SEQ ID NO: 10, the LCDR2 comprises the sequence of SEQ ID NO: 11, and the LCDR3 comprises the sequence of SEQ ID NO: 12; or
b) the HCDR1 comprises the sequence of SEQ ID NO: 13, the HCDR2 comprises the sequence of SEQ ID NO: 14, and the HCDR3 comprises the sequence of SEQ ID NO: 15, the LCDR1 comprises the sequence of SEQ ID NO: 16, the LCDR2 comprises the sequence of SEQ ID NO: 17, and the LCDR3 comprises the sequence of SEQ ID NO: 18.

In one aspect, the present disclosure provides an anti-hVEGFR2 antibody or an antigen-binding fragment thereof provided herein, wherein
a) the HCDR1 comprises the sequence of SEQ ID NO: 1, the HCDR2 comprises the sequence of SEQ ID NO: 2, and the HCDR3 comprises the sequence of SEQ ID NO: 3, the LCDR1 comprises the sequence of SEQ ID NO: 4, the LCDR2 comprises the sequence of SEQ ID NO: 5, and the LCDR3 comprises the sequence of SEQ ID NO: 6; or
b) the HCDR1 comprises the sequence of SEQ ID NO: 19, the HCDR2 comprises the sequence of SEQ ID NO: 20, and the HCDR3 comprises the sequence of SEQ ID NO: 21, the LCDR1 comprises the sequence of SEQ ID NO: 22, the LCDR2 comprises the sequence of SEQ ID NO: 23, and the LCDR3 comprises the sequence of SEQ ID NO: 24.

In certain embodiments, the antibodies provided herein comprise one or more (e.g. 1, 2, 3, 4, 5, or 6) CDR sequences of a hVEGFR2 antibodies 002, 003, 006, 018, 042, 048 and 054.

“002” or “2” as used herein with respect to an antibody refers to a mouse antibody having a heavy chain variable region of SEQ ID NO: 50, and a light chain variable region of SEQ ID NO: 51.

“003” or “3” as used herein with respect to an antibody refers to a mouse antibody having a heavy chain variable region of SEQ ID NO: 52, and a light chain variable region of SEQ ID NO: 53.

“006” or “6” as used herein with respect to an antibody refers to a mouse antibody having a heavy chain variable region of SEQ ID NO: 54, and a light chain variable region of SEQ ID NO: 55.

“018” or “18” as used herein with respect to an antibody refers to a mouse antibody having a heavy chain variable region of SEQ ID NO: 56, and a light chain variable region of SEQ ID NO: 57.

“042” or “42” as used herein with respect to an antibody refers to a mouse antibody having a heavy chain variable region of SEQ ID NO: 58, and a light chain variable region of SEQ ID NO: 59.

“048” or “48” as used herein with respect to an antibody refers to a mouse antibody having a heavy chain variable region of SEQ ID NO: 60, and a light chain variable region of SEQ ID NO: 61.

“054” or “45” as used herein with respect to an antibody refers to a mouse antibody having a heavy chain variable region of SEQ ID NO: 62, and a light chain variable region of SEQ ID NO: 63.

Table 1 shows the CDR sequences of these hVEGFR2 antibodies. The heavy chain and light chain variable region sequences are also provided below in Table 2.

TABLE 1
Sequences of hVEGFR2 antibodies' CDR region

| | Region | CDR1 | CDR2 | CDR3 |
|---|---|---|---|---|
| 002 | HCDR | SEQ ID NO: 1 | SEQ ID NO: 2 | SEQ ID NO: 3 |
| | | SSWMN | RIFPGDGDTYYNGK | FLDTSGRYVDY |
| | | | FQV | |
| | LCDR | SEQ ID NO: 4 | SEQ ID NO: 5 | SEQ ID NO: 6 |
| | | KASQDVNTAVA | SASYRYI | QQHYRAPLT |
| 003 | HCDR | SEQ ID NO: 7 | SEQ ID NO: 8 | SEQ ID NO: 9 |
| | | TYWIM | DIYPGTGSTNYNEKF | DSNPDY |
| | | | KS | |
| | LCDR | SEQ ID NO: 10 | SEQ ID NO: 11 | SEQ ID NO: 12 |
| | | RASESVDNSGISFMT | AASTQGS | QQSKEVPYT |
| 006 | HCDR | SEQ ID NO: 13 | SEQ ID NO: 14 | SEQ ID NO: 15 |
| | | SYWIM | DIYPGSGSTNYNEKF | DSNPDY |
| | | | KS | |
| | LCDR | SEQ ID NO: 16 | SEQ ID NO: 17 | SEQ ID NO: 18 |
| | | RASESVENSGISFMH | AASYQRS | QQSKEVPYT |
| 018 | HCDR | SEQ ID NO: 19 | SEQ ID NO: 20 | SEQ ID NO: 21 |
| | | DYYMS | FIRNKANGYTTEYSA | FDYYGSTYCFDY |
| | | | SVKG | |
| | LCDR | SEQ ID NO: 22 | SEQ ID NO: 23 | SEQ ID NO: 24 |
| | | RASQSVSTSSSSFMH | YASNLES | QHTWEIPLT |
| 042 | HCDR | SEQ ID NO: 25 | SEQ ID NO: 26 | SEQ ID NO: 27 |
| | | IYGMS | SISVGGSYTYYADSV | ELDGNYDY |
| | | | EG | |
| | LCDR | SEQ ID NO: 28 | SEQ ID NO: 29 | SEQ ID NO: 30 |
| | | RSSKSLLYKDGKTYL | LMSTRAS | QQLVEYPFT |
| | | N | | |
| 048 | HCDR | SEQ ID NO: 31 | SEQ ID NO: 32 | SEQ ID NO: 33 |
| | | MYGMS | SISIGGSYTYYADSVE | EMDGNYDY |
| | | | G | |
| | | | SEQ ID NO: 37 | |
| | | | SISIGGSYTYYADSVK | |
| | | | G | |
| | LCDR | SEQ ID NO: 28 | SEQ ID NO: 29 | SEQ ID NO: 30 |
| | | RSSKSLLYKDGKTYL | LMSTRAS | QQLVEYPFT |
| | | N | | |
| 054 | HCDR | SEQ ID NO: 34 | SEQ ID NO: 35 | SEQ ID NO: 36 |
| | | MYGMS | SISIGGSYTYYADSVE | ELDGNYDY |
| | | | G | |
| | | | SEQ ID NO: 37 | |
| | | | SISIGGSYTYYADSVK | |
| | | | G | |
| | LCDR | SEQ ID NO: 28 | SEQ ID NO: 29 | SEQ ID NO: 30 |
| | | RSSKSLLYKDGKTY | LMSTRAS | QQLVEYPFT |
| | | LN | | |
| 042 & 048 | HCDR | SEQ ID NO: 41 | SEQ ID NO: 42 | SEQ ID NO: 43 |
| & 054 | | X₁YGMS | SISX₂GGSYTYYADSV | EX₃DGNYDY |
| | | | X₁₉G | |
| | LCDR | SEQ ID NO: 28 | SEQ ID NO: 29 | SEQ ID NO: 30 |
| | | RSSKSLLYKDGKTYL | LMSTRAS | QQLVEYPFT |
| | | N | | |
| 003 & 006 | HCDR | SEQ ID NO: 44 | SEQ ID NO: 45 | SEQ ID NO: 46 |
| | | X₄YWIM | DIYPGX₅GSTNYNEK | DSNPDY |
| | | | FKS | |
| | LCDR | SEQ ID NO: 47 | SEQ ID NO: 48 | SEQ ID NO: 49 |
| | | RASESVX₆NSGISFMX₇ | AASX₈QX₉S | QQSKEVPYT |

wherein X₁ is I or M, X₂ is V or I, X₃ is L or M, X₄ is T or S, X₅ is T or S, X₆ is D or E, X₇ is T or H, X₈ is T or Y, X₉ is G or R, and X₁₉ is E or K.

TABLE 2
Sequences of mouse/chimeric antibody VH/VL

| | VH | VL |
|---|---|---|
| 002 | SEQ ID NO: 50 | SEQ ID NO: 51 |
| | QVQLQQSGPELVKPGASVKISCKASGYAFSSS | DIVMTQSHKFMSTSVGDRVSITCKASQDVNT |
| | WMNWVKQRPGEGLEWIGRIFPGDGDTYYN | AVAWYQQKPGQSPKLLIYSASYRYIGVPDRFT |
| | GKFQVKATLTADKSSSTAYMQLSSLTSEDSAV | GSGSGTDFTFTISSVQSEDLTVYYCQQHYRAP |
| | YFCAIFLDTSGRYVDYWGQGTTLTISS | LTFGSGTKLELK |
| 003 | SEQ ID NO: 52 | SEQ ID NO: 53 |
| | QVQLQQPGAELVKPGASVKMSCKASGYTFN | DIVLTQSPASLAVSLGQRATISCRASESVDNSG |
| | TYWIMWVKQRPGQGLEWIGDIYPGTGSTNY | ISFMTWFQQKPGQPPKLLIYAASTQGSGVPA |
| | NEKFKSKVTLTADTSSTTAYMQVSSLTSEDSA | RFSGSGSGTDFSLNIHPVEEDDTAMYFCQQSK |
| | VYYCGRDSNPDYWGQGTTLTVSS | EVPYTFGGGTKLEIK |
| 006 | SEQ ID NO: 54 | SEQ ID NO: 55 |
| | QAQLQQPGAELVKPGTSVKMSCKASGYTENS | DIVLTQSPASLAVSLGQRATISCRASESVENSG |
| | YWIMWVKQSPGQGLEWIGDIYPGSGSTNYN | ISFMHWFQQKPGQPPKLLIYAASYQRSGVPA |
| | EKFKSKVTLTVDTSSSTAYMQVSSLTSEDSAV | RFSGSGSGTDFSLNIHPVEEDDIAMYFCQQSK |
| | YYCARDSNPDYWGQGTTLTVSS | EVPYTFGGGTKLEIK |
| 018 | SEQ ID NO: 56 | SEQ ID NO: 57 |
| | EVKLVESGGGLVQPGGSLSLSCAVSGFTFTDY | DIVLTQSPASLAVSLGQRATISCRASQSVSTSS |
| | YMSWVRQPPGKALEWLGFIRNKANGYTTE | SSFMHWYQQKPGQPPKLLIKYASNLESGVPA |
| | YSASVKGRFTISRDNSQSILYLQMNALRAEDS | RFSGSGSGTDFTLNIHPVEEEDTATYYCQHT |
| | ATYYCARFDYYGSTYCFDYWGQGTTLTVSS | WEIPLTFGAGTKLELK |
| 042 | SEQ ID NO: 58 | SEQ ID NO: 59 |
| | EVQLVESGGDLVKPGGSLKLSCTASGFSFSIY | DIVITQNELSNPVTFGESVSISCRSSKSLLYKD |
| | GMSWVRQTPDKRLEWVASISVGGSYTYYAD | GKTYLNWFLQRPGQSPQLLIYLMSTRASGVS |
| | SVEGRFTISRENAKNTLYLQMNSLKSEDTALY | DRFSGSGSGTDFTLEISRVKAEDVGVYYCQQ |
| | YCARELDGNYDYWGQGTSLTVSS | LVEYPFTFGSGTKLEIK |
| 048 | SEQ ID NO: 60 | SEQ ID NO: 61 |
| | EVQLVESGGDLVKPGGSLKLSCAASGFTFSM | DVMITQDELSNPVTFGESVSISCRSSKSLLYK |
| | YGMSWVRQTPDKRLEWVASISIGGSYTYYA | DGKTYLNWFLQRPGQSPQLLIYLMSTRASG |
| | DSVEGRFTISRENAKNTLFLQMNSLKSEDTAL | VSDRFSGSGSGTDFTLEISRVKAEDVGIYYCQ |
| | YYCAREMDGNYDYWGHGTTLTVSS | QLVEYPFTFGSGTKLEIK |
| 054 | SEQ ID NO: 62 | SEQ ID NO: 63 |
| | EVQLVESGGDLVKPGGSLKLSCAASGFTFSM | HIMITQDELSNPVTFGESVSISCRSSKSLLYKD |
| | YGMSWVRQTPDKRLEWVASISIGGSYTYYA | GKTYLNWFLQRPGQSPQLLIYLMSTRASGVS |
| | DSVEGRFIISRENAKNTLFLQMNSLKSEDTAL | DRFSGSGSGTDFTLEISRVKAEDVGIYYCQQL |
| | YYCARELDGNYDYWGQGTTLTVSS | VEYPFTFGSGTKLEIK |

wherein the CDR regions are bolded.

The anti-hVEGFR2 antibodies or antigen-binding fragments thereof provided herein can be a monoclonal antibody, polyclonal antibody, humanized antibody, chimeric antibody, recombinant antibody, bispecific antibody, labeled antibody, bivalent antibody, or anti-idiotypic antibody. A recombinant antibody is an antibody prepared in vitro using recombinant methods rather than in animals.

CDRs are known to be responsible for antigen binding, however, it has been found that not all of the 6 CDRs are necessarily indispensable or unchangeable. In other words, it is possible to replace or change or modify 1, 2, or 3 CDRs in anti-hVEGFR2 antibodies provided herein, yet substantially retain the specific binding affinity to hVEGFR2.

In certain embodiments, the anti-hVEGFR2 antibodies and the antigen-binding fragments provided herein comprise a heavy chain CDR3 sequence of one of the anti-hVEGFR2 antibodies 002, 003, 006, 018, 042, 048 and 054. Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, etc. J Mol Biol. 263:551-67).

In some embodiments, the anti-hVEGFR2 antibodies and the antigen-binding fragments provided herein comprise all or a portion of the heavy chain variable domain and/or all or a portion of the light chain variable domain. In one embodiment, the anti-hVEGFR2 antibodies and the antigen-binding fragments provided herein is a single domain antibody which consists of all or a portion of the heavy chain variable domain provided herein. More information of such a single domain antibody is available in the art (see, e.g., U.S. Pat. No. 6,248,516).

In certain embodiments, the antibodies and antigen-binding fragments thereof provided herein comprise suitable framework region (FR) sequences, as long as the antibodies and antigen-binding fragments thereof can specifically bind to hVEGFR2. The CDR sequences provided in Table 1 are obtained from mouse antibodies, but they can be grafted to any suitable FR sequences of any suitable species such as mouse, human, rat, rabbit, among others, using suitable methods known in the art such as recombinant techniques.

In certain embodiments, the antibodies and antigen-binding fragments thereof provided herein are humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536).

Suitable human heavy chain and light chain variable domains can be selected to achieve this purpose using methods known in the art. In an illustrative example, “best-fit” approach can be used, where a non-human (e.g., rodent) antibody variable domain sequence is screened or BLASTed against a database of known human variable domain germline sequences, and the human sequence closest to the non-human query sequence is identified and used as the human scaffold for grafting the non-human CDR sequences (see, for example, Sims et al, (1993) J. Immunol. 151:2296; Chothia et al. (1987) J. Mot. Biol. 196:901). Alternatively, a framework derived from the consensus sequence of all human antibodies may be used for the grafting of the non-human CDRs (see, for example, Carter et al. (1992) Proc. Natl. Acad. Sci. USA, 89:4285; Presta et al. (1993) J. Immunol., 151:2623).

In certain embodiments, the humanized antibodies or antigen-binding fragments provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived different human immunoglobulin genes, for example, FR sequences derived from one human antibody and constant region from another human antibody. In some embodiments, the humanized antibody or antigen-binding fragment comprise human heavy/light chain FR1-4.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure to reduce or avoid immunogenicity and/or improve or retain the binding activity or binding affinity.

In certain embodiments, the humanized antibody or antigen-binding fragment provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FRs of a heavy or a light chain variable domain. In some embodiments, such change in amino acid residue could be present in heavy chain FR regions only, in light chain FR regions only, or in both chains. In certain embodiments, the one or more amino acid residues are mutated, for example, back-mutated to the corresponding residue found in the non-human parent antibody (e.g. in the mouse framework region) from which the CDR sequences are derived. Suitable positions for mutations can be selected by a skilled person following principles known in the art. For example, a position for mutation can be selected where: 1) the residue in the framework of the human germline sequence is rare (e.g. in less than 20% or less than 10% in human variable region sequence); 2) the position is immediately adjacent to one or more of the 3 CDR's in the primary sequence of the human germline chain, as it is likely to interact with residues in the CDRs; or 3) the position is close to CDRs in a 3-dimensional model, and therefore can have a good probability of interacting with amino acids in the CDR. The residue at the selected position can be mutated back to the corresponding residue in the parent antibody, or to a residue which is neither the corresponding residue in human germline sequence nor in parent antibody, but to a residue typical of human sequences, i.e. that occurs more frequently at that position in the known human sequences belonging to the same subgroup as the human germline sequence (see U.S. Pat. No. 5,693,762).

In certain embodiments, the humanized light and heavy chains of the present disclosure are substantially non-immunogenic in humans and retain substantially the same affinity as or even higher affinity than the parent antibody to hVEGFR2.

In certain embodiments, the humanized antibodies and antigen-binding fragment thereof provided herein comprise one or more light chain FR sequences of human germline framework sequence VK/2D-40, and/or one or more heavy chain FR sequences of human germline framework sequence V_{H}/3-21, with or without back mutations. Back mutations can be introduced into the human germline framework sequence, if needed. In certain embodiments, the humanized antibody 054 may contain one or more back mutations selected from the group consisting of: R19K, A40T, G44R, S49A, S78T, and R87K relative to the framework sequences in SEQ ID NO: 92

(EVQLVESGGGLVKPGGSLRLSCAASGFTFSSYSMNWVRQAPGKGLEWVSSISSSSSYIYYADSVKGR FTISRDNAKNSLYLQMNSLPAEDTAVYYCARELDGNYDYWGQGTTLTVSS) of the heavy chain framework sequence V_{H}/3-21. The humanized antibody 048 may contain one or more back mutations selected from the group consisting of: G44R, S49A and S78T relative to the framework sequences in SEQ ID NO: 92
(EVQLVESGGGLVKPGGSLRLSCAASGFTFSSYSMNWVRQAPGKGLEWVSSISSSSSYIYYADSVKGR FTISRDNAKNSLYLQMNSLPAEDTAVYYCARELDGNYDYWGQGTTLTVSS) of the heavy chain framework sequence V_{H}/3-21. In certain embodiments, the humanized antibody 054/048 may contain one or more back mutations selected from the group consisting of: M4T, T7D, P8E, P15F, P18S, A19V, Y42F, K45R, P65S and V91I relative to the framework sequences in SEQ ID NO: 95
(DIVMTQTPLSLPVTPGEPAS ISCRSSQSLLDSDDGNTYLDWYLQKPGQSPQLLIYTLSYPASGVPDR FSGSGSGTDFTLKISRVEAEDVGVYYCMQRIEFP) of the light chain framework sequence VK/2D-40.

In certain embodiments, the anti-hVEGFR2 antibody or an antigen-binding fragment thereof provided herein, comprises a heavy chain variable region comprising the sequence selected from the group consisting of SEQ ID NO: 93, SEQ ID NO: 94 and SEQ ID NO: 98, and a homologous sequence thereof having at least 80% (e.g. at least 85%, 90%, 95%, 96%, 97%, 98%, or 99%) sequence identity yet retaining specific binding affinity to hVEGFR2, in particular human hVEGFR2.

In certain embodiments, the anti-hVEGFR2 antibody or an antigen-binding fragment thereof provided herein, antibody or an antigen-binding fragment thereof comprises a light chain variable region comprising the sequence selected from the group consisting of SEQ ID NO: 96 and SEQ ID NO: 97, and a homologous sequence thereof having at least 80% (e.g. at least 85%, 90%, 95%, 96%, 97%, 98%, or 99%) sequence identity yet retaining specific binding affinity to hVEGFR2, in particular human hVEGFR2.

In certain embodiments, the anti-hVEGFR2 antibody or an antigen-binding fragment thereof provided herein, comprising: a heavy chain variable region comprising the sequence selected from the group consisting of SEQ ID NO: 93, SEQ ID NO: 94 and SEQ ID NO: 98, and a light chain variable region comprising the sequence of SEQ ID NO: 96 or SEQ ID NO: 97.

In certain embodiments, the anti-hVEGFR2 antibody or an antigen-binding fragment thereof provided herein further comprises one or more of heavy chain HFR1, HFR2, HFR3 and HFR4, and/or one or more of light chain LFR1, LFR2, LFR3 and LFR4, wherein:
the HFR1 comprises EVQLVESGGGLVKPGGSLXioLSCAASGFTFS (SEQ ID NO: 84) or a homologous sequence of at least 80% (or at least 85%, 90%, 95%) sequence identity thereof,
the HFR2 comprises WVRQX₁₁PGKRLEWVA (SEQ ID NO: 85) or a homologous sequence of at least 80% (or at least 90%) sequence identity thereof,
the HFR3 sequence comprises RFTISRDNAKNTLYLQMNSLX₁₂AEDTAVYYCAR (SEQ ID NO: 86) or a homologous sequence of at least 80% (or at least 85%, 90%, 95%) sequence identity thereof,
the HFR4 comprises WGX₁₃GTTLTVSS (SEQ ID NO: 87) or a homologous sequence of at least 80% sequence identity thereof,
the LFR1 comprises DIVITQX₁₄X15LSLPVTX₁₆GESVSISC (SEQ ID NO: 88) or a homologous sequence of at least 80% (or at least 85%, 90%, 95%) sequence identity thereof,
the LFR2 comprises WFLQRPGQSPQLLIY (SEQ ID NO: 89) or a homologous sequence of at least 80% (or at least 85%, 90%) sequence identity thereof,
the LFR3 comprises GVX₁₇DRFSGSGSGTDFTLKISRVEAEDVGX₁₈YYC (SEQ ID NO: 90) or a homologous sequence of at least 80% (or at least 85%, 90%, 95%) sequence identity thereof, and
the LFR4 comprises FGSGTKLEK (SEQ ID NO: 91) or a homologous sequence of at least 8000 (or at least 900LG) sequence identity thereof,
wherein X₁₀ is R or K, X₁₁ is A or T, X₁₂ is R or K, X₁₃ is Q or H, X₁₄ is D or T, X₁₅ is E or P, X₁₆ is For P, X₁₇ is S or P, X₁₈ is V or I.

In certain embodiments, the HIFR1 comprises a sequence selected from the group consisting of SEQ ID NOs: 64, 68 and 72, the HFR2 comprises a sequence selected from the group consisting of SEQ ID NOs: 65, 69 and 73, the HFR3 comprises the sequence selected from the group consisting of SEQ ID NOs: 66, 70 and 74, the HFR4 comprises a sequence selected from the group consisting of SEQ ID NOs: 67, 71 and 75, the LFR1 comprises the sequence from the group consisting of SEQ ID NOs: 76 and 80, the LFR2 comprises a sequence selected from the group consisting of SEQ ID NO: 77 and 81, the LFR3 comprises a sequence selected from the group consisting of SEQ ID NOs: 78 and 82, and the LFR4 comprises a sequence selected from the group consisting of SEQ ID NOs: 79 and 83.

TABLE 3
Framework (FR) sequences of humanized hVEGFR2 antibodies 054 and 048

| Antibody chain | FR1 | FR2 | FR3 | FR4 |
|---|---|---|---|---|
| Mab54-Hzd-HC-V1 | SEQ ID NO: 64 | SEQ ID NO: 65 | SEQ ID NO: 66 | SEQ ID NO: 67 |
| | EVQLVESGGGL | WVRQTPGKRLE | RFTISRDNAKNT | WGQGTTLTVSS |
| | VKPGGSLKLSC | WVA | LYLQMNSLKAE | |
| | AASGFTFS | | DTAVYYCAR | |
| Mab54-HC-V2 | SEQ ID NO: 68 | SEQ ID NO: 69 | SEQ ID NO: 70 | SEQ ID NO: 71 |
| | EVQLVESGGGL | WVRQAPGKRLE | RFTISRDNAKNT | WGQGTTLTVSS |
| | VKPGGSLRLSCA | WVA | LYLQMNSLRAE | |
| | ASGFTFS | | DTAVYYCAR | |
| Mab48-Hzd-HC-V3 | SEQ ID NO:72 | SEQ ID NO: 73 | SEQ ID NO: 74 | SEQ ID NO: 75 |
| | EVQLVESGGGL | WVRQAPGKRLE | RFTISRDNAKNT | WGHGTTLTVSS |
| | VKPGGSLRLSCA | WVA | LYLQMNSLRAE | |
| | ASGFTFS | | DTAVYYCAR | |
| Mab54-Hzd-LC-V1 | SEQ ID NO: 76 | SEQ ID NO: 77 | SEQ ID NO: 78 | SEQ ID NO: 79 |
| | DIVITQDELSLPV | WFLQRPGQSPQ | GVSDRFSGSGSG | FGSGTKLEIK |
| | TFGESVSISC | LLIY | TDFTLKISRVEA | |
| | | | EDVGIYYC | |
| Mab54-Hzd-LC-V2 | SEQ ID NO: 80 | SEQ ID NO: 81 | SEQ ID NO: 82 | SEQ ID NO: 83 |
| | DIVITQTPLSLPV | WFLQRPGQSPQ | GVPDRFSGSGSG | FGSGTKLEIK |
| | TPGESVSISC | LLIY | TDFTLKISRVEA | |
| | | | EDVGVYYC | |

Tables 4 and 5 illustrate sequences of the variable regions of humanized 054 and 048 antibodies.

TABLE 4
Sequences of humanized 054

| Antibody chain | Sequences |
|---|---|
| Mab54-germline | EVQLVESGGGLVKPGGSLRLSCAASGFTFSSYSMNWVRQAPGKGLEWVSSISS |
| | SSSYIYYADSVKGRFTISRDNAKNSLYLQMNSLRAEDTAVYYCARELDGNYDY |
| | WGQGTTLTVSS (SEQ ID NO: 92) |
| Mab54-Hzd-HC-V1 | EVQLVESGGGLVKPGGSLKLSCAASGFTFSMYGMSWVRQTPGKRLEWVASISI |
| | GGSYTYYADSVKGRFTISRDNAKNTLYLQMNSLKAEDTAVYYCARELDGNYD |
| | YWGQGTTLTVSS (SEQ ID NO: 93) |
| Mab54-HC-V2 | EVQLVESGGGLVKPGGSLRLSCAASGFTFSMYGMSWVRQAPGKRLEWVASISI |
| | GGSYTYYADSVKGRFTISRDNAKNTLYLQMNSLRAEDTAVYYCARELDGNYD |
| | YWGQGTTLTVSS (SEQ ID NO: 94) |
| Mab54 LC germline | DIVMTQTPLSLPVTPGEPASISCRSSQSLLDSDDGNTYLDWYLQKPGQSPQLLIY |
| | TLSYRASGVPDRFSGSGSGTDFTLKISRVEAEDVGVYYCMQRIEFP (SEQ ID |
| | NO: 95) |
| Mab54-Hzd-LC-V1 | DIVITQDELSLPVTFGESVSISCRSSKSLLYKDGKTYLNWFLQRPGQSPQLLIYL |
| | MSTRASGVSDRFSGSGSGTDFTLKISRVEAEDVGIYYCQQLVEYPFTFGSGTKL |
| | EIK (SEQ ID NO: 96) |
| Mab54-Hzd-LC-V2 | DIVITQTPLSLPVTPGESVSISCRSSKSLLYKDGKTYLNWFLQRPGQSPQLLIYLM |
| | STRASGVPDRFSGSGSGTDFTLKISRVEAEDVGVYYCQQLVEYPFTFGSGTKLEI |
| | K (SEQ ID NO: 97) |

TABLE 5
Sequences of humanized 048

| Antibody chain | Sequences |
|---|---|
| Mab48-Hzd-HC-V3 | EVQLVESGGGLVKPGGSLRLSCAASGFTFSMYGMSWVRQAP |
| | GKRLEWVASISIGGSYTYYADSVKGRFTISRDNAKNTLYLQM |
| | NSLRAEDTAVYYCAREMDGNYDYWGHGTTLTVSS (SEQ ID |
| | NO: 98) |

The light chain variable regions of the humanized 048 are the same of those of the humanized 054.

The humanized anti-hVEGFR2 antibodies provided herein retained the specific binding affinity to hVEGFR2-expressing cell, and are at least comparable to, or even better than, the parent antibodies in that aspect. The humanized antibodies provided herein can also retain their functional interaction with VEGFR2-expressing cells, such as HUVEC cells, in that all antibodies can inhibit VEGF-A induced VEGFR2 phosphorylation, HUVEC proliferation and tube formation. In certain embodiments, the anti-hVEGFR2 antibodies and the fragments thereof provided herein further comprise an immunoglobulin constant region, optionally a constant region of human Ig, or optionally a constant region of human IgG. In some embodiments, an immunoglobulin constant region comprises a heavy chain and/or a light chain constant region. The heavy chain constant region comprises CH1, hinge, and/or CH2-CH3 regions. In certain embodiments, the heavy chain constant region comprises an Fc region. In certain embodiments, the light chain constant region comprises Cκ or Cλ.

In certain embodiments, the anti-hVEGFR2 antibodies and the fragments thereof provided herein further comprise a constant region of human IgG1, IgG2, IgG3, or IgG4. In certain embodiments, the anti-hVEGFR2 antibodies and antigen-binding fragments thereof provided herein comprises a constant region of IgG1 isotype. In certain embodiments, the constant region of human IgG1 comprises SEQ ID NO: 38

(ASTKGPSVFPLAPSSKSTSGGTAALGCLVKDYFPEPVTVSWNSGALTSGVHTFPAVLQSSGLYSLSS VVTVPSSSLGTQTYICNVNHKPSNTKVDKKVEPKSCDKTHTCPPCPAPEAAGGPSVFLFPPKPKDTL MISRTPEVTCVVVDVSHEDPEVKFNWYVDGVEVHNAKTKPREEQYNSTYRVVSVLTVLHQDWLNGKE YKCKVSNKALPAPIEKTISKAKGQPREPQVYTLPPSREEMTKNQVSLTCLVKGFYPSDIAVEWESNG QPENNYKTTPPVLDSDGSFFLYSKLTVDKSRWQQGNVFSCSVMHEALHNHYTQKSLSLSPGK), or a homologous sequence having at least 80% (e.g. at least 85%, 90%, 95%, 96%, 97%, 98%, or 99%) sequence identity thereof.

Constant region of IgG1 isotype can induce effector functions such as ADCC or CDC. Effector functions of the anti-hVEGFR2 antibodies and the antigen-binding fragments thereof provided herein can lead to cytotoxicity to cells expressing hVEGFR2. Effector functions can be evaluated using various assays such as Fc receptor binding assay, C1q binding assay, and cell lysis assay, and any of the assays described above for determining ADCC or CDC.

### Antibody Variants

The anti-hVEGFR2 antibodies and antigen-binding fragments thereof provided herein also encompass various types of variants of the antibody sequences provided herein.

In certain embodiments, the variants comprise one or more modification(s) or substitution(s) in 1, 2, or 3 CDR sequences as provided in Table 1, in one or more FR sequences, in the heavy or light chain variable region sequences provided herein, and/or in the constant region (e.g., Fc region). Such antibody variants retain specific binding affinity to hVEGFR2 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, reduced or increased effector function(s), improved FcRn receptor binding, increased pharmacokinetic half-life, pH sensitivity, and/or compatibility to conjugation (e.g., one or more introduced cysteine residues), to name a few.

A parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g., cysteine residue, positively charged residue, etc.).

### 1. Affinity Variant

An affinity variant retain specific binding affinity to hVEGFR2 of the parent antibody, or even have improved hVEGFR2 specific binding affinity over the parent antibody. Various methods known in the art can be used to achieve this purpose. For example, a library of antibody variants (such as Fab or scFv variants) can be generated and expressed with phage display technology, and then screened for the binding affinity to hVEGFR2. For another example, computer software can be used to virtually simulate the binding of the antibodies to hVEGFR2, and identify the amino acid residues on the antibodies which form the binding interface. Such residues may be either avoided in the substitution so as to prevent reduction in binding affinity, or targeted for substitution to provide for a stronger binding.

In certain embodiments, at least one (or all) of the substitution(s) in the CDR sequences, FR sequences, or variable region sequences comprises a conservative substitution. A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among residues with neutral hydrophilic side chains (e.g., Cys, Ser, Thr, Asn and Gln), among residues with acidic side chains (e.g., Asp, Glu), among amino acids with basic side chains (e.g., His, Lys, and Arg), or among residues with aromatic side chains (e.g., Trp, Tyr, and Phe). As known in the art, conservative substitution usually does not cause significant change in the protein conformational structure, and therefore could retain the biological activity of a protein.

In certain embodiments, the antibody or antigen-binding fragment provided herein comprises one or more amino acid residue substitutions in one or more CDR sequences, and/or one or more FR sequences. In certain embodiments, an affinity variant comprises no more than 10, 9, 8, 7, 6, 5, 4, 3, 2, or 1 substitutions in one or more of the CDR sequences and/or FR sequences in total.

In certain embodiments, the anti-hVEGFR2 antibodies and antigen-binding fragments thereof comprise 1, 2, or 3 CDR sequences having at least 80% (e.g., at least 85%, 88%, 90%, 91%, 92%, 93%, 94%, 95%, 96%, 97%, 98%, 99%) sequence identity to that (or those) listed in Table 1, and in the meantime retain the binding affinity to hVEGFR2 at a level similar to or even higher than its parental antibody.

In certain embodiments, the anti-hVEGFR2 antibodies and antigen-binding fragments thereof comprise one or more variable region sequences having at least 80% (e.g., at least 85%, 88%, 90%, 91%, 92%, 93%, 94%, 95%, 96%, 97%, 98%, 99%) sequence identity to that (or those) of SEQ ID NOs: 50-63, 93-94 and 96-98, and in the meantime retain the binding affinity to hVEGFR2 at a level similar to or even higher than its parent antibody. In some embodiments, a total of 1 to 10 amino acids have been substituted, inserted, or deleted in a sequence selected from SEQ ID NOs: 50-63, 93-94 and 96-98. In some embodiments, the substitutions, insertions, or deletions occur in regions outside the CDRs (i.e., in the FRs).

### 2. Glycosylation Variant

The anti-hVEGFR2 antibodies and antigen-binding fragments provided herein also encompass a glycosylation variant, which can be obtained to either increase or decrease the extent of glycosylation of the antibody or antigen binding fragment. The term “glycosylation” as used herein, refers to enzymatic process that attaches glycans such as fucose, xylose, mannose, or GlcNAc phosphoserine glycan to proteins, lipids, or other organic molecules. Depending on the carbon linked to the glycan, glycosylation can be divided into five classes including: N-linked glycosylation, O-linked glycosylation, phospho-glycosylation, C-linked glycosylation, and glypiation.

Glycosylation of antibodies is typically N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine.

In certain embodiments, the antibody or antigen-binding fragment thereof provided herein is afucosylated. The term “afucosylation,” or “afucosylated,” refers to the reduced or eliminated core-fucose on the N-glycan attached to the antibody. The majority glycans of human IgG antibodies are known as G0, G1 and G2, which are complex biantennary molecules with core fucose residue carrying zero, one or two terminal galactose.

Afucosylated antibody variants can be made using methods known in the art, for example, as described in US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004).

In certain embodiments, the antibody glycosylation variants can be obtained by, for example, removal of a native glycosylation site (e.g. by N297A substitution), such that tripeptide sequences for N-linked glycosylation sites or serine or threonine residues for O-linked glycosylation sites no longer present in the antibody or Fc sequence. Alternatively, in certain embodiments, antibody glycosylation variants can be obtained by producing the antibody in a host cell line that is defective in adding the selected sugar group(s) to the mature core carbohydrate structure in the antibody.

### 3. Cysteine-Engineered Variant

The anti-hVEGFR2 antibodies and antigen-binding fragments provided herein also encompass a cysteine-engineered variant, which comprises one or more introduced free cysteine amino acid residues.

A free cysteine residue is one which is not part of a disulfide bridge. A cysteine-engineered variant is useful for conjugation with, for example a cytotoxic and/or imaging compound, a label, or a radioisoptype among others, at the site of the engineered cysteine, through for example a maleimide or haloacetyl. Methods for engineering antibodies or antigen-binding fragments to introduce free cysteine residues are known in the art, see, for example, WO2006/034488.

### Antigen-Binding Fragments

Provided herein are also anti-hVEGFR2 antigen-binding fragments. Various types of antigen-binding fragments are known in the art and can be developed based on the anti-hVEGFR2 antibodies provided herein, including for example, the exemplary antibodies whose CDR sequences are shown in Tables 1, and their different variants (such as affinity variants, glycosylation variants, Fc variants, cysteine-engineered variants and so on).

In certain embodiments, an anti-hVEGFR2 antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

Various techniques can be used for the production of such antigen-binding fragments. Illustrative methods include, enzymatic digestion of intact antibodies (see, e.g., Morimoto et al., Journal of Biochemical and Biophysical Methods 24:107-117 (1992); and Brennan et al., Science, 229:81 (1985)), recombinant expression by host cells such as E. Coli (e.g., for Fab, Fv and ScFv antibody fragments), screening from a phage display library as discussed above (e.g., for ScFv), and chemical coupling of two Fab′-SH fragments to form F(ab′)₂ fragments (Carter et al., Bio/Technology 10:163-167 (1992)). Other techniques for the production of antibody fragments will be apparent to a skilled practitioner.

In certain embodiments, the antigen-binding fragment is a scFv. Generation of scFv is described in, for example, WO 93/16185; U.S. Pat. Nos. 5,571,894; and 5,587,458. scFv may be fused to an effector protein at either the amino or the carboxyl terminus to provide for a fusion protein (see, for example, Antibody Engineering, ed. Borrebaeck).

In certain embodiments, the anti-hVEGFR2 antibodies and antigen-binding fragments thereof provided herein are bivalent, tetravalent, hexavalent, or multivalent. The term “valent” as used herein refers to the presence of a specified number of antigen binding sites in a given molecule. As such, the terms “bivalent”, “tetravalent”, and “hexavalent” denote the presence of two binding site, four binding sites, and six binding sites, respectively, in an antigen-binding molecule. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e. having the same sequences), or structurally different (i.e. having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope).

### Bispecific Antibodies

In certain embodiments, the antibodies and antigen-binding fragments thereof provided herein are bispecific. The term “bispecific” as used herein encompasses molecules having more than two specificity and molecules having more than two specificity, i.e. multispecific. In certain embodiments, the bispecific antibodies and antigen-binding fragments thereof provided herein is capable of specifically binding to a first and a second epitopes of hVEGFR2, or capable of specifically binding to hVEGFR2 and a second antigen. In certain embodiments, the first epitope and the second epitopes of hVEGFR2 are distinct from each other or non-overlapping. In certain embodiments, the bispecific antibodies and antigen-binding fragments thereof can bind to both the first epitope and the second epitope at the same time.

In certain embodiments, the bispecific antibody comprises a first binding domain and a second binding domain, wherein the first binding domain comprises a HCDR1 comprising the sequence selected from the group consisting of SEQ ID NOs: 25, 31, 34 and 1, a HCDR2 comprising the sequence selected from the group consisting of SEQ ID NOs: 26, 32, 35 and 2, a HCDR3 comprising the sequence selected from the group consisting of SEQ ID NOs: 27, 33, 36 and 3, a LCDR1 comprising the sequence selected from the group consisting of SEQ ID NOs: 28 and 4, a LCDR2 comprising the sequence selected from the group consisting of SEQ ID NOs: 29 and 5, and a LCDR3 comprising the sequence selected from the group consisting of SEQ ID NOs: 30 and 6; and wherein the second binding domain comprises a HCDR1 comprising the sequence selected from the group consisting of SEQ ID NOs: 7, 13 and 19, a HCDR2 comprising the sequence selected from the group consisting of SEQ ID NOs: 8, 14 and 20, a HCDR3 comprising the sequence selected from the group consisting of SEQ ID NOs: 9, 15 and 21, a LCDR1 comprising the sequence selected from the group consisting of SEQ ID NOs: 10, 16 and 22, a LCDR2 comprising the sequence selected from the group consisting of SEQ ID NOs: 11, 17 and 23, and a LCDR3 comprising the sequence selected from the group consisting of SEQ ID NOs: 12, 18 and 24.

In certain embodiments, the bispecific antibody comprises a first binding domain and a second binding domain,
wherein the first binding domain comprising:
a) a HCDR1 comprises the sequence of SEQ ID NO: 25, a HCDR2 comprising the sequence of SEQ ID NO: 26, a HCDR3 comprising the sequence of SEQ ID NO: 27; a LCDR1 comprising the sequence of SEQ ID NO: 28, a LCDR2 comprising the sequence of SEQ ID NO: 29, and a LCDR3 comprising the sequence of SEQ ID NO: 30; or
b) a HCDR1 comprising the sequence of SEQ ID NO: 31, a HCDR2 comprising the sequence of SEQ ID NO: 32 or SEQ ID NO: 37, a HCDR3 comprising the sequence of SEQ ID NO: 33, a LCDR1 comprising the sequence of SEQ ID NO: 28, a LCDR2 comprising the sequence of SEQ ID NO: 29, and a LCDR3 comprising the sequence of SEQ ID NO: 30; or
c) a HCDR1 comprising the sequence of SEQ ID NO: 34, a HCDR2 comprising the sequence of SEQ ID NO: 35 or SEQ ID NO: 37, a HCDR3 comprising the sequence of SEQ ID NO: 36, a LCDR1 comprising the sequence of SEQ ID NO: 28, a LCDR2 comprising the sequence of SEQ ID NO: 29, and a LCDR3 comprising the sequence of SEQ ID NO: 30; or
d) a HCDR1 comprising the sequence of SEQ ID NO: 1, a HCDR2 comprising the sequence of SEQ ID NO: 2, a HCDR3 comprising the sequence of SEQ ID NO: 3, a LCDR1 comprising the sequence of SEQ ID NO: 4, a LCDR2 comprising the sequence of SEQ ID NO: 5, and a LCDR3 comprising the sequence of SEQ ID NO: 6; and wherein the second binding domain comprising:
e) a HCDR1 comprising the sequence of SEQ ID NO: 7, a HCDR2 comprising the sequence of SEQ ID NO: 8, a HCDR3 comprising the sequence of SEQ ID NO: 9; a LCDR1 comprising the sequence of SEQ ID NO: 10, a LCDR2 comprising the sequence of SEQ ID NO: 11, and a LCDR3 comprising the sequence of SEQ ID NO: 12; or
f) a HCDR1 comprising the sequence of SEQ ID NO: 13, a HCDR2 comprising the sequence of SEQ ID NO: 14, a HCDR3 comprising the sequence of SEQ ID NO: 15, a LCDR1 comprising the sequence of SEQ ID NO: 16, a LCDR2 comprising the sequence of SEQ ID NO: 17, and a LCDR3 comprising the sequence of SEQ ID NO: 18, or
g) a HCDR1 comprising the sequence of SEQ ID NO: 19, a HCDR2 comprising the sequence of SEQ ID NO: 20, a HCDR3 comprising the sequence of SEQ ID NO: 21, a LCDR1 comprising the sequence of SEQ ID NO: 22, a LCDR2 comprising the sequence of SEQ ID NO: 23, and a LCDR3 comprising the sequence of SEQ ID NO: 24.

In certain embodiments, the second antigen is different from hVEGFR2.

In certain embodiments, the second antigen is an immune related target. In some embodiments, the bispecific antibodies and antigen-binding fragments thereof specifically bind to hVEGFR2 and an immune related target, and are capable of targeting the immune cells to hVEGFR2-expressing cells (e.g. hVEGFR2-expressing tumor cells), and/or activating hVEGFR2 specific immune response to the hVEGFR2-expressing target cells. An immune related target as used herein, encompasses a biological molecule that is involved in the generation or modulation of an immune response, optionally, cellular immune responses. An example of the immune related target is immune checkpoint molecule, and a surface molecule of a cytolytic immune cell such as T cell or natural killer (NK) cell.

Immune checkpoint molecule can mediate co-stimulatory signal to augment immune response, or can mediate co-inhibitory signals to suppress immune response. Examples of an immune checkpoint molecule include, for example, PD-L1, PD-L2, PD-1, CLTA-4, TIM-3, LAG3, A2AR, CD160, 2B4, TGF β, VISTA, BTLA, TIGIT, LAIR1, OX40, CD2, CD27, CD28, CD30, CD40, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-15, CD3, CD16 and CD83.

Cytolytic immune cells can be triggered by its surface molecule to attack and mediate lysis of a target cell such as a tumor cell. In certain embodiments, the second antigen is a T cell surface antigen. Examples of a T cell surface antigen include, without limitation, an antigen selected from the group consisting of CD3, CD2, CD4, CD5, CD6, CD8, CD28, CD40L and/or CD44, preferably CD3. In certain embodiments, said second antigen is the epsilon-chain of CD3. In certain embodiments, binding of said bispecific antibody to CD3 on T cells results in proliferation and/or activation of said T cells, which induces release of cytotoxic factors, e.g. perforins and granzymes, and cytolysis and apoptosis of the target cells. In certain embodiments, the second antigen is a NK cell surface antigen, such as CD16 (FcγRIII) or CD56. In certain embodiments, binding of bispecific antibody to CD16 on NK cells leads to NK-cell degranulation and perforin-dependent target cell lysis (ADCC) of the target cells.

In certain embodiments, the second antigen comprises a tumor antigen. “Tumor antigen” as used herein refers to tumor specific antigens (e.g. those unique to tumor cells and normally not found on non-tumor cells), tumor-associated antigens (e.g. found in both tumor and non-tumor cells but expressed differently in tumor cells), and tumor neo-antigens (e.g. that are expressed in cancer cells because of somatic mutations that change the protein sequence or create fusion proteins between two unrelated sequences).

Examples of tumor antigens include, without limitation, EpCAM, HER2/neu, HER3/neu, C250, CEA, MAGE, proteoglycans, VEGF, EGFR, αVβ3-integrin, HLA, HLA-DR, ASC, CD1, CD2, CD4, CD6, CD7, CD8, CD11, CD13, CD14, CD19, CD20, CD21, CD22, CD23, CD24, CD30, CD33, CD37, CD40, CD41, CD47, CD52, c-erb-2, CALLA, MHCII, CD44v3, CD44v6, p97, ganglioside GM1, GM2, GM3, GDla, GD1b, GD2, GD3, GT1 b, GT3, GQ1, NY-ESO-1, NFX2, SSX2, SSX4, Trp2, gp100(Pmel 17), tyrosinase, Muc-1, telomerase, survivin, G250, p53, CA125 MUC, Wue antigen, Lewis Y antigen, HSP-27, HSP-70, HSP-72, HSP-90, Pgp, MCSP, EpHA2 and cell surface targets GC1 82, GT468 or GT512, PD-L1, arboviral E protein epitope, glioma-associated antigen, carcinoembryonic antigen (CEA), 3-human chorionic gonadotropin, alphafetoprotein (AFP), lectin-reactive AFP, thyroglobulm, RAGE-1, MN-CA IX, human telomerase reverse transcriptase, RU1, RU2 (AS), intestinal carboxyi esterase, mut hsp70-2, M-CSF, prostase, prostate-specific antigen (PSA), PAP, NY-ESO-1, LAGE-la, p53, prostein, PSMA, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin, ART-1/MelanA (MART-1), tyrosinase, TRP-1, TRP-2 and tumor-specific multilineage antigens such as MAGE-1, MAGE-3, BAGE, GAGE-1, GAGE-2, pi 5; Ras, unique tumor antigens resulting from chromosomal translocations; such as BCR-ABL, E2A-PRL, H4-RET, 1GH-IGK, MYL-RAR; and viral antigens, such as the Epstein Barr virus antigens EBVA and the human papillomavirus (HPV) antigens E6 and E7; protein-based antigens include TSP-180, MAGE-4, MAGE-5, MAGE-6, RAGE, NY-ESO, pl 85erbB2, pl 80erbB-3, c-met, nm-23H 1, PSA, TAG-72, CA19-9, CA72-4, CAM 17.1, NuMa, K-ras, beta-Catenin, CDK4, Mum-1, p15, p16, 43-9F, 5T4(791Tgp72), a-fetoprotem, beta-HCG, BCA225, BTAA, CA 125, CA 15-3CA 27.29BCAA, CA 195, CA 242, CA-50, CAM43, CD68I, CO-029, FGF-5, G250, Ga733VEpCAM, HTgp-175, M344, MA-50, MG7-Ag, MOV 18, NB/70K, NY-CO-1, RCAS 1, SDCCAG16, TA-90Mac-2 binding protein, cyclophilin C-associated protein, TAAL6, TAG72, TLP, and TPS.

Bispecific antibodies and antigen-binding fragments thereof provided herein can be in a suitable format known in the art. For example, an exemplary bispecific format can be, bispecific diabodies, scFv-based bispecific formats, IgG-scFv fusions, dual variable domain (DVD)-Ig, Quadroma, knobs-into-holes, common light chain (e.g., common light chain with knobs-into-holes, etc.), BiTE, CrossMab, CrossFab, Duobody, SEEDbody, leucine zipper, dual acting Fab (DAF)-IgG, and Mab² bispecific formats (see, e.g., Brinkmann et al. 2017, Mabs, 9(2): 182-212). The bispecific molecules can be in symmetric or asymmetric architecture.

The bispecific antibodies and antigen-binding fragments provided herein can be made with any suitable methods known in the art.

In one embodiment, two immunoglobulin heavy chain-light chain pairs having different antigenic specificities are co-expressed in a host cell to produce bispecific antibodies in a recombinant way (see, for example, Milstein and Cuello, Nature, 305: 537 (1983)), followed by purification by affinity chromatography.

In another embodiment, sequences encoding the antibody heavy chain variable domains for the two specificities are respectively fused to immunoglobulin constant domain sequences, followed by insertion to one or more expression vector(s) which is/are co-transfected with an expression vector for the light chain sequences to a suitable host cell for recombinant expression of the bispecific antibody (see, for example, WO 94/04690; Suresh et al., Methods in Enzymology, 121:210 (1986)). Similarly, scFv dimers can also be recombinantly constructed and expressed from a host cell (see, e.g., Gruber et al., J. Immunol., 152:5368 (1994).)

In another method, leucine zipper peptides from the Fos and Jun. proteins can be linked to the Fab′ portions of two different antibodies by gene fusion. The linked antibodies are reduced at the hinge region to four half antibodies (i.e. monomers) and then re-oxidized to form heterodimers (Kostelny et al., J. Immunol., 148(5):1547-1553 (1992)).

The two antigen-binding domains may also be conjugated or cross-linked to form a bispecific antibody or antigen-binding fragment. For example, one antibody can be coupled to biotin while the other antibody to avidin, and the strong association between biotin and avidin would complex the two antibodies together to form a bispecific antibody (see, for example, U.S. Pat. No. 4,676,980; WO 91/00360, WO 92/00373, and EP 03089). For another example, the two antibodies or antigen-binding fragments can be cross-linked by conventional methods known in the art, for example, as disclosed in U.S. Pat. No. 4,676,980.

Bispecific antigen-binding fragments may be generated from a bispecific antibody, for example, by proteolytic cleavage, or by chemical linking. For example, an antigen-binding fragment (e.g., Fab′) of an antibody may be prepared and converted to Fab′-thiol derivative and then mixed and reacted with another converted Fab′ derivative having a different antigenic specificity to form a bispecific antigen-binding fragment (see, for example, Brennan et al., Science, 229: 81 (1985)).

In certain embodiments, the bispecific antibody or antigen-binding fragments thereof provided herein may be engineered at the interface so that a knob-into-hole association can be formed to promote heterodimerization of the two different antigen-binding sites. This can maximize the percentage of heterodimers which are recovered from recombinant cell culture. “Knob-into-hole” as used herein, refers to an interaction between two polypeptides (such as Fc), where one polypeptide has a protuberance (i.e. “knob”) due to presence of an amino acid residue having a bulky side chain (e.g., tyrosine or tryptophan), and the other polypeptide has a cavity (i.e. “hole”) where a small side chain amino acid residue resides (e.g., alanine or threonine), and the protuberance is positionable in the cavity so as to promote interaction of the two polypeptides to form a heterodimer or a complex. Methods of generating polypeptides with knobs-into-holes are known in the art, e.g., as described in U.S. Pat. No. 5,731,168.

### Conjugates

In some embodiments, the anti-hVEGFR2 antibodies and antigen-binding fragments thereof are linked to one or more conjugate moieties. A conjugate is a moiety that can be attached to the antibody or antigen-binding fragment thereof. It is contemplated that a variety of conjugates may be linked to the antibodies or antigen-binding fragments provided herein (see, for example, “Conjugate Vaccines”, Contributions to Microbiology and Immunology, J. M. Cruse and R. E. Lewis, Jr. (eds.), Carger Press, New York, (1989)). These conjugates may be linked to the antibodies or antigen-binding fragments by covalent binding, affinity binding, intercalation, coordinate binding, complexation, association, blending, or addition, among other methods. In certain embodiments, the antibodies or antigen binding fragments thereof are linked to one or more conjugates via a linker. In certain embodiments, the linker is a hydrazone linker, a disulfide linker, a bifunctional linker, dipeptide linker, glucuronide linker, a thioether linker.

In certain embodiments, the anti-hVEGFR2 antibodies and antigen-binding fragments disclosed herein may be engineered to contain specific sites outside the epitope binding portion that may be utilized for binding to one or more conjugates. For example, such a site may include one or more reactive amino acid residues, such as for example cysteine or histidine residues, to facilitate covalent linkage to a conjugate.

The conjugate can be a clearance-modifying agent, therapeutic agent (e.g., a chemotherapeutic agent), a toxin, a radioactive isotope, a detectable label (e.g., a lanthanide, a luminescent label, a fluorescent label, or an enzyme-substrate label), a pharmacokinetic modifying moiety, a DNA-alkylators, a topoisomerase inhibitor, a tubulin-binders, other anticancer drugs, or a purifying moiety (such as a magnetic bead or nanoparticle).

Examples of detectable label may include a fluorescent labels (e.g., fluorescein, rhodamine, dansyl, phycoerythrin, or Texas Red), enzyme-substrate labels (e.g., horseradish peroxidase, alkaline phosphatase, luceriferases, glucoamylase, lysozyme, saccharide oxidases or P-D-galactosidase), radioisotuopes, other lanthanides, luminescent labels, chromophoric moiety, digoxigenin, biotin/avidin, a DNA molecule or gold for detection.

Examples of radioisotopes may include ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P. Radioisotope labelled antibodies are useful in receptor targeted imaging experiments.

In certain embodiments, the conjugate can be a pharmacokinetic modifying moiety such as PEG which helps increase half-life of the antibody. Other suitable polymers include, such as, carboxymethylcellulose, dextran, polyvinyl alcohol, polyvinyl pyrrolidone, copolymers of ethylene glycol/propylene glycol, and the like.

In certain embodiments, the conjugate can be a purification moiety such as a magnetic bead or a nanoparticle.

### Antibody-Drug Conjugates

In certain embodiments, the present disclosure provides antibody-drug conjugates (ADC) comprising any of the above anti-hVEGFR2 antibodies or antigen-binding fragments conjugated to a cytotoxic agent.

ADC can be useful for local delivery of cytotoxic agents, for example, in the treatment of cancer. This allows for targeted delivery of cytotoxic agents to tumors and intracellular accumulation therein, which is particularly useful where systemic administration of these unconjugated cytotoxic agents may result in unacceptable levels of toxicity to normal cells as well as the tumor cells sought to be eliminated (Baldwin et al., (1986) Lancet pp. (Mar. 15, 1986):603-05; Thorpe, (1985) “Antibody Carriers Of Cytotoxic Agents In Cancer Therapy: A Review,” in Monoclonal Antibodies '84: Biological And Clinical Applications, A. Pinchera et al. (ed.s), pp. 475-506; Syrigos and Epenetos (1999) Anticancer Research 19:605-614; Niculescu-Duvaz and Springer (1997) Adv. Drg Del. Rev. 26:151-172; U.S. Pat. No. 4,975,278).

In certain embodiments, the cytotoxic agent can be any agent that is detrimental to cells or that can damage or kill cells. In certain embodiments, the cytotoxic agent is optionally a toxin, a chemotherapeutic agent (such as a DNA-alkylators, a topoisomerase inhibitor, a tubulin-binders, a growth inhibitory agent, or other anticancer drugs), or a radioactive isotope.

Examples of toxins include bacterial toxins and plant toxins, such as for example, diphtheria toxin, exotoxin A chain (from Pseudomonas aeruginosa), ricin, abrin, modeccin, alpha-sarcin, Aleurites fordii. proteins, dianthin proteins, Phytolaca americana proteins (PARI, PAPII, and PAP-S), Momordica charantia inhibitor, curcin, crotin, Sapaonaria officinalis inhibitor, gelonin, restrictocin, phenomycin, enomycin, and the tricothecenes (see, e.g., WO 93/21232). Such a large molecule toxin can be conjugated to the antibodies or antigen-binding fragments provided herein using methods known in the art, for example, as described in Vitetta et al (1987) Science, 238:1098.

The cytotoxic agent can also be small molecule toxins and chemotherapeutic agents, such as geldanamycin (Mandler et al (2000) Jour. of the Nat. Cancer Inst. 92(19):1573-1581; Mandler et al (2002) Bioconjugate Chem. 13:786-791), maytansine and maytansinoids (EP 1391213; Liu et al., (1996) Proc. Natl. Acad. Sci. USA 93:8618-8623; U.S. Pat. No. 5,208,020), calicheam icin (Lode et al (1998) Cancer Res. 58:2928; Hinman et al (1993) Cancer Res. 53:3336-3342), taxol, cytochalasin B, gramicidin D, ethidium bromide, emetine, mitomycin, etoposide, tenoposide, vincristine, vinblastine, vindesine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine), calicheamicin, maytansinoids, dolastatins, auristatins such as MMAE and MMAF (U.S. Pat. Nos. 5,635,483; 5,780,588), dolostatins, a trichothecene, and CC1065, and the derivatives thereof having cytotoxic activity.

The cytotoxic agent can also be a highly radioactive isotope. Examples include At ²¹¹ I¹³¹, I¹²⁵, Y⁹⁰, Re¹⁸⁶, Sm¹⁵³, Bi²¹², P³², Pb²¹² and radioactive isotopes of Lu. Methods of conjugation of a radioisotope to an antibody is known in the art, for example, via a suitable ligand reagent (see, e.g., WO94/11026; Current Protocols in Immunology, Volumes 1 and 2, Coligen et al, Ed. Wiley-Interscience, New York, N.Y, Pubs. (1991)). A ligand reagent has a chelating ligand that can bind, chelate or otherwise complex a radioisotope metal, and also has a functional group that is reactive with a thiol of cysteine of an antibody or antigen-binding fragment. Exemplary chelating ligands include DOTA, DOTP, DOTMA, DTPA and TETA (Macrocyclics, Dallas, Tex.).

The cytotoxic agents can be linked to an antibody or antigen-binding fragment via any suitable linkers known in the art, see, for example, in U.S. Pat. Nos. 5,208,020, 6,441,163, or EP Patent 0 425 235 B1, Chari et al., Cancer Research 52:127-131 (1992), and US 2005/0169933 A1, the disclosures of which are hereby expressly incorporated by reference.

In certain embodiments, the linker is cleavable under a particular physiological environment, thereby facilitating release of the cytotoxic drug in the cell. For example, the linker can be an acid-labile linker, peptidase-sensitive linker, photolabile linker, dimethyl linker or disulfide-containing linker, thioether linker, and esterase labile linker (Chari et al., Cancer Research 52:127-131(1992); U.S. Pat. No. 5,208,020). In some embodiments, the linker may comprise amino acid residues, such as a dipeptide, a tripeptide, a tetrapeptide or a pentapeptide. The amino acid residues in the linker may be natural or non-naturally occurring amino acid residues. Examples of such linkers include: valine-citrulline (ve or val-cit), alanine-phenylalanine (af or ala-phe), glycine-valine-citrulline (gly-yal-cit), glycine-glycine-glycine (gly-gly-gly), an valine-citrullin-p-aminobenzyloxycaronyl (“vc-PAB”). Amino acid linker components can be designed and optimized in their selectivity for enzymatic cleavage by a particular enzymes, for example, a tumor-associated protease, cathepsin B, C and D, or a plasmin protease.

In certain embodiments, the cytotoxic agents can be linked to the antibody or antigen-binding fragment thereof provided herein by a bifunctional linker reagent include, such as N-succinimidyl-3-(2-pyridyldithio) propionate (SPDP), succinimidyl-4-(N-maleimidomethyl) cyclohexane-1-carboxylate (SMCC), N-succinimidyl-4-(2-pyridylthio)pentanoate (SPP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), bis-active fluorine compounds (such as 1,5-difluom-2,4-dinitrobenzene), BMPS, EMCS, GMBS, HBVS, LC-SMCC, MBS, MPRH, SBAP, SIA, SIAB, SMPB, SMPH, sulfo-EMCS, sulfo-GMBS, sulfo-KMUS, sulfo-MBS, sulfo-SIAB, sulfo-SMCC, and sulfo-SMPB, and SVSG (succinimidyl-(4-vinylsulfone)benzoate). Those linker reagents are commercially available (e.g., from Pierce Biotechnology, Inc., Rockford, Ill., USA, see pages 467-498, 2003-2004 Applications Handbook and Catalog).

In certain embodiments, in the ADC provided herein, an antibody (or antigen-binding fragment thereof) is conjugated to one or more cytotoxic agents at an antibody: agent ratio of about 1 to about 20, about 1 to about 6, about 2 to about 6, about 3 to about 6, about 2 to about 5, about 2 to about 4, or about 3 to about 4.

The ADC provided herein may be prepared by any suitable methods known in the art. In certain embodiments, a nucleophilic group of the antibody (or antigen-binding fragment thereof) is first reacted with a bifunctional linker reagent and then linked to the cytotoxic agent, or the other way around, i.e., first reacting a nucleophilic of the cytotoxic agent with a bifunctional linker and then linking to the antibody.

In certain embodiments, the cytotoxic agent may contain (or modified to contain) a thiol reactive functional group which may react with a cysteine thiol of a free cysteine of the antibodies or antigen-binding fragments provided herein. Exemplary thiol-reactive functional group include, for example, a maleimide, an iodoacetamide, a pyridyl disulfide, haloacetyl, succinimidyl ester (e.g., NHS, N-hydroxysuccinimide), isothiocyanate, sulfonyl chloride, 2,6-dichlorotriazinyl, pentafluorophenyl ester, or phosphoramidite (Haugland, 2003, Molecular Probes Handbook of Fluorescent Probes and Research Chemicals, Molecular Probes, Inc.; Brinkley, 1992, Bioconjugate Chem. 3:2; Garman, 1997, Non-Radioactive Labelling: A Practical Approach, Academic Press, London; Means (1990) Bioconjugate Chem. 1:2; Hermanson, G. in Bioconjugate Techniques (1996) Academic Press, San Diego, pp. 40-55, 643-671).

The cytotoxic agent or the antibody may react with a linking reagent before being conjugated to form the ADC. For example, N-hydroxysuccinimidyl ester (NHS) of a cytotoxic agent may be performed, isolated, purified, and/or characterized, or it may be formed in situ and reacted with a nucleophilic group of an antibody. Typically, the carboxyl form of the conjugate is activated by reacting with some combination of a carbodiimide reagent, e.g., dicyclohexylcarbodiimide; diisopropyl carbodiimide, or a uronium reagent, e.g., TsTu (O-(N-Succinimidyl)-N,N,N′,N′-tetramethyluronium tetrafluoroborate, HBTU (O-benzotriazol-1-yl)-N,N,N′N′-tetramethyluronium hexafluorophosphate), or HATU (0-(7-azabenzotriazol-1-yl)-N,N,N′,N′-tetramethyluronium hexafluorophosphate), an activator, such as 1-hydroxybenzotriazole (HOBt), and N-hydroxysuccinimide to give the NHS ester. In some cases, the cytotoxic agent and the antibody may be linked by in situ activation and reaction to form the ADC in one step. Other activating and linking reagents include TBTU (2-(1H-benzotriazo-1-yl)-1-1,3,3-tetramethyluronium hexafluorophosphate), TFFH (N,N′,N″,N′″-tetramethyluronium 2-fluoro-hexafluorophosphate), PyBOP (benzotriazole-1-yl-oxy-tris-pyrrolidino-phosphonium hexafluorophosphate, EEDQ (2-ethoxy-1-ethoxycarbonyl-1,2-dihydro-quinoline), DCC (dicyclohexylcarbodiimide); DIPCDI (diisopropylcarbodiimide), MSNT (1-(mesitylene-2-sulfonyl)-3-nitro-1H-1,2,4-triazole, and aryl sulfonyl halides, e.g., triisopropylbenzenesulfonyl chloride. In another example, the antibody or antigen-binding fragments may be conjugated to biotin, then indirectly conjugated to a second conjugate that is conjugated to avidin.

### Polynucleotides and Recombinant Methods

The present disclosure provides isolated polynucleotides that encode the anti-hVEGFR2 antibodies and antigen-binding fragments thereof. The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

DNA encoding the monoclonal antibody is readily isolated and sequenced using conventional procedures (e.g. by using oligonucleotide probes that are capable of binding specifically to genes encoding the heavy and light chains of the antibody). The encoding DNA may also be obtained by synthetic methods.

The present disclosure provides vectors (e.g. expression vectors) comprising the isolated polynucleotide provided herein. In certain embodiments, the expression vector provided herein comprises the polynucleotide encoding the antibodies or antigen-binding fragments thereof provided herein, at least one promoter (e.g. SV40, CMV, EF-la) operably linked to the polynucleotide sequence, and at least one selection marker. Examples of vectors include, but are not limited to, retrovirus (including lentivirus), adenovirus, adeno-associated virus, herpesvirus (e.g. herpes simplex virus), poxvirus, baculovirus, papillomavirus, papovavirus (e.g. SV40), lambda phage, and M13 phage, plasmids such as pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pProl8, pTD, pRS10, pLexA, pACT2.2, pCMV-SCRIPT.RTM., pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g. E. coli, Enterobacter, Erwinia, Klebsiella, Proteus, Salmonella, e.g. Salmonella typhimurium, Serratia, e.g. Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-hVEGFR2 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g. K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g. Neurospora, Penicillium, Tolypocladium, and Aspergillus hosts such as A. nidulans and A. niger.

Suitable host cells for the expression of glycosylated antibodies or antigen-fragment provided herein are derived from multicellular organisms such as invertebrate cells, for example plant and insect cells. Numerous baculoviral strains and variants and corresponding permissive insect host cells from hosts such as Spodopterafrugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g. the L-1 variant of Autographa californica NPV and the Bm-5 strain of Bombyx mori NPV, and such viruses may be used as the virus herein according to the present invention, particularly for transfection of Spodopterafrugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some preferable embodiments, the host cell is a mammalian cultured cell line, such as CHO, BHK, NS0, 293 and their derivatives. In some preferable embodiments, the host cell is CHO and its derivatives.

Host cells are transformed with the above-described expression or cloning vectors for anti-hVEGFR2 antibody production and cultured in conventional nutrient media modified as appropriate for inducing promoters, selecting transformants, or amplifying the genes encoding the desired sequences. In another embodiment, the antibody may be produced by homologous recombination known in the art.

The host cells used to produce the antibodies or antigen-binding fragments provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-hVEGFR2 antibodies and antigen-binding fragments thereof prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)).

Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX.TM. resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Pharmaceutical Composition

The present disclosure further provides pharmaceutical compositions comprising the anti-hVEGFR2 antibodies or antigen-binding fragments thereof and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment and conjugates as provided herein decreases oxidation of the antibody or antigen-binding fragment. This reduction in oxidation prevents or reduces loss of binding affinity, thereby improving antibody stability and maximizing shelf-life. Therefore, in certain embodiments compositions are provided that comprise one or more antibodies or antigen-binding fragments as disclosed herein and one or more antioxidants such as methionine. Further provided are methods for preventing oxidation of, extending the shelf-life of, and/or improving the efficacy of an antibody or antigen-binding fragment as provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-hVEGFR2 antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### Methods of Use

The present disclosure also provides therapeutic methods comprising: administering a therapeutically effective amount of the antibody or antigen-binding fragment as provided herein and/or the pharmaceutical composition provided herein to a subject in need thereof, thereby treating, reducing the severity of and/or slowing the progression of a VEGFR2-related disease or condition in a subject.

In some embodiments, the VEGFR2-related disease or condition is a tumor or an angiogenic disease.

### Tumors

In certain embodiments, the VEGFR2-related disease or condition is a tumor, such as a solid tumor or non-solid tumor. In some embodiments, the tumor produces VEGF (e.g., VEGF-A) and/or is sensitive to VEGF (e.g., VEGF-A) present in its microenvironment. Studies have observed VEGF production and VEGFR2 expression in solid tumors or non-solid tumors, such as human leukemias (Sato, K. et al., Tohoku J. Exp. Med., 185: 173-84 (1998); Ishii, Y, Nippon Sanka Fujinka Gakkai Zasshi: 4′I: 133-40 (1995); and Ferrer, F. A. et al, Urology, 54:567-72 (1999); Fielder et al., Blood 89:1870-5 (1997) and Bellamy et al., Cancer Res. 59728-33 (1999)). Without wishing to be bound by theory, the VEGF/hVEGFR2 autocrine loop modulates tumor cell survival and migration in vivo and it has been further demonstrated that VEGFR1 monoclonal antibodies inhibited the autocrine VEGF/VEGFR1 loop in certain solid tumor cells, e.g., breast carcinoma cells, and inhibited the VEGF-stimulated migration of human leukemia cells (see, e.g., U.S. Pat. No. 7,498,414(B2)).

In certain embodiments, the solid tumor is selected from the group consisting of breast carcinoma, lung carcinoma, colorectal carcinoma, pancreatic carcinoma, glioma and lymphoma, for example, head and neck tumors, colorectal tumors, prostate tumors, breast tumors, lung tumors, such as small cell and non-small cell lung tumors, pancreatic tumors, thyroid tumors, ovarian tumors, cervical tumors, kidney tumors, brain tumors, and liver tumors, Kaposi's sarcoma, CNS neoplasms, neuroblastomas, capillary hemangioblastomas, meningiomas, cerebral metastases, melanoma, gastrointestinal and renal carcinomas and sarcomas (e.g., gastric cancer), rhabdomyosarcoma, glioblastoma, preferably glioblastoma multiforme, leiomyosarcoma, squamous cell carcinoma, basal cell carcinoma and skin cancers that can be treated by suppressing the growth of malignant keratinocytes, such as human malignant keratinocytes. In certain embodiments, the solid tumor is selected from the group consisting of gastric cancer, non-small cell lung cancer, such as large cell lung cancer.

In certain embodiments, the non-solid tumor is selected from the group consisting of leukemia, multiple myeloma and lymphoma, for example, acute myelogenous leukemia (AML), chronic myelogenous leukemia (CML), acute lymphocytic leukemia (ALL), chronic lymphocytic leukemia (CLL), erythrocytic leukemia ormonocytic leukemia, Hodgkin's and non-Hodgkin's lymphoma.

### Angiogenic Diseases

In certain embodiments, the VEGFR2-related disease or condition is an angiogenic disease. Angiogenic diseases are associated with uncontrolled angiogenesis, which can be regulated by VEGF, e.g., VEGF-A. Expression of VEGF can be found in embryonic tissues, macrophages, and proliferating epidermal keratinycytes during wound healing (Breier et al., Development, 114:521-32 (1992); Brown et al., J. Exp. Med., 176:1375-9 (1992); and Ferrara et al., Endocr. Rev, 13:18-32 (1992)). High expression of VEGF can also be found in tumors, such as glioblastoma multiforme, hemangioblastoma, central nervous system neoplasms and AIDS-associated Kaposi's sarcoma; in atheroscelerotic lesions, plaques and in inflammatory cells (Nakamura, S. et al., AIDS Weekly, 13 (1) (1992); Plate, K. et al., Nature, 359:845-8 (1992); Plate, K. et al., Cancer Res., 53:5822-7 (1993); and Berkman, R. et al., J. Clin. Invest., 91:153-9 (1993)). Without wishing to be bound by theory, endothelial cells adjacent to tumor cells expressing VEGF generally show up-regulated expression of VEGF receptors, e.g., VEGFR2. Release of VEGF by tumors leads to stimulation of angiogenesis in adjacent endothelial cells, which results in proliferation, migration, differentiation, tube formation, maintenance of vascular integrity and increase in vascular permeability of the endothelial cells. Therefore, the abnormalities in the VEGF/VEGFR2 signaling involved in angiogenic diseases can be treated by the anti-VEGFR2 antibodies or antigen-binding fragments thereof provided by the present disclosure.

In some embodiments, the angiogenic disease is atherosclerosis, rheumatoid arthritis (RA), neovascular glaucoma, proliferative retinopathy including proliferative diabetic retinopathy, macular degeneration, hemangiomas, angiofibromas, psoriasis, retinopathy of prematurity (e.g., retrolental fibroplastic), corneal graft rejection, insulin-dependent diabetes mellitus, multiple sclerosis, myasthenia gravis, Chron's disease, autoimmune nephritis, primary biliary cirrhosis, acute pancreatitis, allograph rejection, allergic inflammation, contact dermatitis and delayed hypersensitivity reactions, inflammatory bowel disease, septic shock, osteoporosis, osteoarthritis, cognition defects induced by neuronal inflammation, Osler-Weber syndrome, restinosis, and fungal, parasitic and viral infections, such as cytomegaloviral infections.

In some embodiments, the subject is human.

The antibody or antigen-binding fragment as provided herein and/or the pharmaceutical composition provided herein can be administered via oral, nasal, intravenous, subcutaneous, sublingual, intratumoral, or intramuscular administration.

In some embodiments, the method provided herein further comprises administering a therapeutically effective amount of a second therapeutic agent, for example, an anti-cancer therapy, optionally the anti-cancer therapy is selected from a chemotherapeutic agent, radiation therapy, an immunotherapy agent, anti-angiogenesis agent (e.g. antagonist of a VEGFR such as VEGFR-1, VEGFR-2, and VEGFR-3), an EGFR antagonist, an PDGFR antagonist, an IGFR antagonist, an NGFR antagonist, an FGFR antagonist, a targeted therapy agent, a cellular therapy agent, a gene therapy agent, a hormonal therapy agent, cytokines, palliative care, surgery for the treatment of cancer (e.g., tumorectomy), one or more anti-emetics, treatments for complications arising from chemotherapy, or a diet supplement for cancer patients (e.g. indole-3-carbinol).

In some embodiments, the present disclosure provides kits comprising the antibody or antigen-binding fragment thereof provided herein, optionally conjugated with a detectable moiety. The kits may be useful in detection of presence or amount of hVEGFR2 in a biological sample, or may be useful in the methods of diagnosis provided herein.

In some embodiments, the present disclosure provides kits comprising the antibody or antigen-binding fragment thereof provided herein and a second therapeutic agent. The kits may be useful in treatment, prevention, and/or amelioration of VEGFR2-related disease or condition.

The present disclosure also provides a method of detecting presence or amount of VEGFR2 in a sample, comprising contacting the sample with the antibody or antigen-binding fragment thereof provided herein, and determining the presence or the amount of VEGFR2 in the sample.

In some embodiments, the present disclosure also provides use of the antibody or antigen-binding fragment thereof provided herein in the manufacture of a medicament for treating, reducing the severity of and/or slowing the progression of a VEGFR2 related disease or condition in a subject.

### Examples

### Example 1: Preparation and Characterization of VEGFR2 Protein

Human VEGFR2/KDR-His: Recombinant Human VEGFR/KDR Protein (hVEGFR2-his, Accession #AAI31823.1) was expressed in human 293 cells (HEK293). Briefly the coding region of the human VEGFR2 gene from Ala20-Glu764 with 6×his tag at C-terminus was used for transfection.

The supernatant was purified using His-tag affinity column. The resulting purified protein was characterized using SDS page gel. The protein was purchased from ACRO Biosystems (Cat #KDR-H5227).

Rhesus macaque VEGFR2/KDR with His tag: Recombinant Rhesus macaque VEGFR2/KDR Protein extracellular domain Ala20 to Glu764 (Accession #XP_014994176.1) was fused with polyhistidine tag at C-terminus and produced in human 293 cells (HEK293). The transfection supernatant from HEK293 cells was purified using His-tag affinity column. The resulting purified protein was characterized using SDS page gel. This protein was purchased from ACRO Biosystems (Cat #VE2-C52H3).

The above VEGFR2 proteins were used in the following experiments.

### Example 2: Antibody Generation

### 1. Antigen Conjugation and Immunization

For immunization, the recombinant hVEGFR2-his protein was conjugated with various MabSpace immune-enhancing peptides. Briefly 2-8 fold molar excess of the peptide was mixed with Sulfo-SMCC (sulfosuccinimidyl 4-[N-maleimidomethyl]cyclohexane-1-carboxylate, Peirce #22322)-activated hVEGFR2 protein and incubated for one hour at room temperature. The reaction was stopped and the conjugated protein was analyzed and QCed using SDS-PAGE gel.

Above conjugated hVEGFR2-his protein was emulsified in a 1:1 ratio using Complete Freund's Adjuvant (Pierce), respectively, and then immunized sub-cutaneously and intraperitoneally into C57B/L6 mice. Additional immunizations were carried out using CpG and Alum to preserve native conformation of the protein. Immunization occurred at least every 2 weeks and anti-serum from the mice was taken after the 1st immunizations for anti-hVEGFR2 titer analysis by ELISA assay.

For determining the serum titer, 20 μl of mouse serum was prepared from each immunized mice. High-binding clear polystyrene 96 well plates (Nunc) were coated with 100 μl/well of a 1p g/ml solution consisting of human VEGFR2-his in high pH coating buffer (0.16% Na2CO3, 0.3% NaHCO₃, pH9.8). The plates were incubated overnight at 4° C., and then washed once on an automatic plate washer using washing buffer PBS+0.1% Tween 20 (Sigma). 200p1 of blocking buffer (PBS+1% BSA+1% Goat serum+0.05% Tween 20) was added to each well and incubated for 2 hours at room temperature. The blocking buffer was then aspirated and 100 μl of serially diluted serum in dilution buffer (PBS+1% BSA+1% Goat serum+0.01% Tween 20) was transferred to each well of the ELISA plate and allowed to incubate for 60 min at room temp. The plates were then washed 3 times using the method described above. 100p/well of solution of HRP conjugated goat anti-mouse Fc antibody (Abcam,Cat #Ab98808) diluted in dilution buffer was then added to each well of the plate. After that the ELISA plates were allowed to incubate for 60 min at RT, the plates were washed 3 times with 250 μl/well washing buffer. Finally, 100 μl/well of TMB was added to each well and the reaction was terminated using 0.64M H₂SO₄. The plates were read on a Thermo Multiscan FC at 450 nM.

### 2. Fusions

Four days prior to fusion, each mouse was boosted intraperitoneally with unconjugated hVEGFR2 protein in PBS. On the fusion day, the spleens were removed aseptically and the organs were processed into a single cell suspension. The red blood cells were lysed and the spleenocytes were washed with DMEM (Gibco). Viable, log-phase growth myeloma cells (SP2/0) were mixed with the murine spleenocytes in a 1:4 ratio. The cells were then washed 2 times before the fusion with PEG.

The post fusion cells were washed with DMEM and suspended in cell growth media supplemented with 10% FBS+HFCS+OPI+1X HAT. 200 μl per well of this cell suspension was plated into 96-well cell culture plates and incubated overnight in a 37° C. humidified 10% CO₂ incubator. The cultures were incubated for 7 days and then the growth media was aspirated out of the wells and exchanged for fresh growth media. Screening of hybridoma supernatants commenced 2-3 days after the media change.

### 3. Antibody Screening by ELISA Assay

Same protocol of determining serum titer above. Briefly, 0.5 μg/ml hVEGFR2-his was coated overnight at 4° C. After wash, 100 μl of hybridoma supernatant was added and allowed completely binding. HRP conjugated goat anti-mouse Fc antibody was then added to detect bound VEGFR2 antibody. Finally, the plates were read on a Thermo Multiscan FC at 450 nM after TMB reaction and H₂SO₄ termination. Cells from the ELISA positive hybridoma wells were subsequently expanded in cell culture for further characterization studies.

### Examples 3: Subcloning of Positive Hybridoma Clones and Small Scale Antibody Production

### 1. Subcloning of Positive Hybridoma Clones

Cells from the ELISA positive hybridoma wells with the desired binding profile and blocking activity were selected and each plated using limited dilution in 96 well plates. These cells were allowed to grow for 7 days. Once the adequate cell mass was reached, supernatant from each well was collected and re-screened for antigen binding ability (see screening in Example 2).

From each 96 well plate, clones with highest antigen binding activity were identified and expanded with limited dilution further into 96-well plates with 2001 of hybridoma growth medium per well. After 7 days, cells from 96-well plates were tested for antigen binding. The subcloning was done more than 2 times. When more than 90 of the wells display positive binding signal, two clones with the highest antigen binding activity were identified and transferred to 24-well plates with medium and was allowed to grow for 2 additional days. Once 24 well plates were confluent, cells were transferred to 6-well plates. After 5 days of incubation, a portion of the cells were frozen down. The remainder of the cells were transferred into a flask and allowed to expand. Once the flasks were confluent, half of cells were frozen down (3 vials per clone) for additional backup. The other half was allowed to expand further in flasks with medium for antibody production. Isotypes were determined using standard methodologies.

### 2. Small Scale Antibody Production

Hybridoma cells were inoculated to roller bottle and cultured for 14 days with 200-300 ml of hybridoma culture medium (Invitrogen). VEGFR2 monoclonal antibodies (mAbs) were purified from hybridoma cell culture as follows. All purification processes were carried out at room temperature. One purification scheme was used to purify various mAbs and used affinity chromatography.

The host cell culture fluid (CCF) was centrifuged to remove cell debris. The CCF supernatant was then filtered, diluted and then loaded onto Protein G chromatography media in the form of a column, Protein G High Performance (Bio-Rad) and equilibrated.

After loading, the Protein G column was washed until the absorbance at 280 nm of the flow-through returned to baseline. The VEGFR2 mAb was then eluted from the column using glycine, pH 2.5 and immediately neutralized by adding 50 μL of a stock solution of 1 M Tris Base per mL of elution volume. The absorbance at 280 nm of the eluate was monitored and fractions containing protein were collected to make the Protein A pool.

Following purification, the VEGFR2 mAbs were formulated in PBS by dialysis using 10,000 MWCO membranes (Pierce Slide-A-Lyzer or dialysis tubing). Following formulation, the VEGFR2 mAbs were filtered.

### Example 4: Binding Analysis of Purified VEGFR2 Antibodies

Same protocol of Example 2 antibody screening by ELISA assay. Briefly, 0.5 μg/ml hVEGFR2-his was coated and serially diluted purified antibodies would bind to coated antigen. Using HRP conjugated goat anti-mouse Fc antibody could detect binding signals of each antibody.

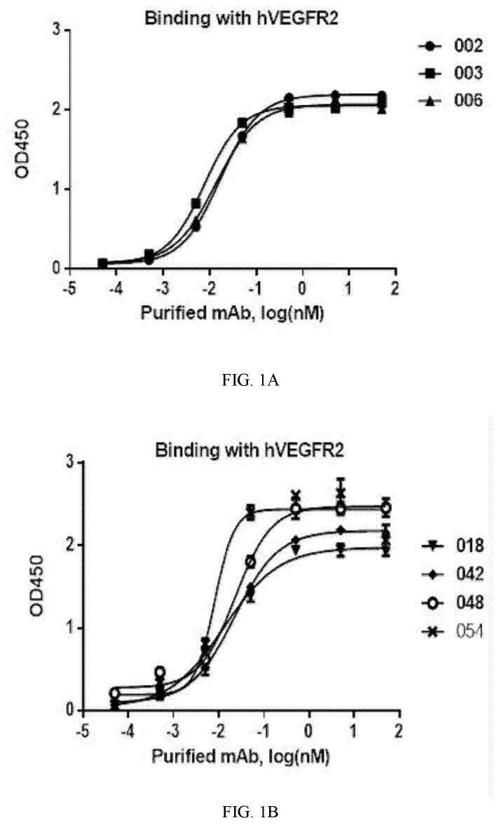

The data were calculated and fit by Graphpad Prism Software and EC50 of antibodies were summarized in FIG. 1A-1B and Table 6. The hybridoma antibodies have similar affinity to human.

TABLE 6
EC50 values ofVEGFR2-specific antibodies
obtained in ELISA binding (ng/ml)

| VEGFR2-specific | |
|---|---|
| hybridoma antibodies | Human VEGFR2 |
| 002 | 16.80 |
| 003 | 7.69 |
| 006 | 13.52 |
| 018 | 12.95 |
| 042 | 22.44 |
| 048 | 23.20 |
| 054 | 8.09 |

### Example 5: Evaluation of Blocking Activities of Purified Antibodies to Inhibit Binding of hVEGF-A to hVEGFR2

High-binding clear polystyrene 96 well plates were coated with 100 μl/well of 1 μg/ml hVEGFR2-his overnight at 4° C. After washing and blocking, serial diluted antibodies (from 10 μg/ml to 0.0006 μg/ml) were added and incubate for 1 hour at RT. 0.3 μg/ml hVEGF-A (Acrobiosystem, Cat #VE5-H4210) were added and incubated for 2 hour at RT. Then 100 μl/well of 0.25 μg/ml Biotinylated Rabbit Anti-human VEGF-A (Peprotech, Cat #400-P10Bt) was then added for detecting bound VEGF-A. Then 100 μl/well of a solution of HRP conjugated Neutravidin antibody (Pierce, Cat #31001) and TMB were added sequentially. Finally, reaction was terminated using 0.64M H₂SO₄ and plates were read at 450 nM. 1121B is a benchmark antibody (Ramucirumab) disclosed in the U.S. Pat. No. 7,498,414.

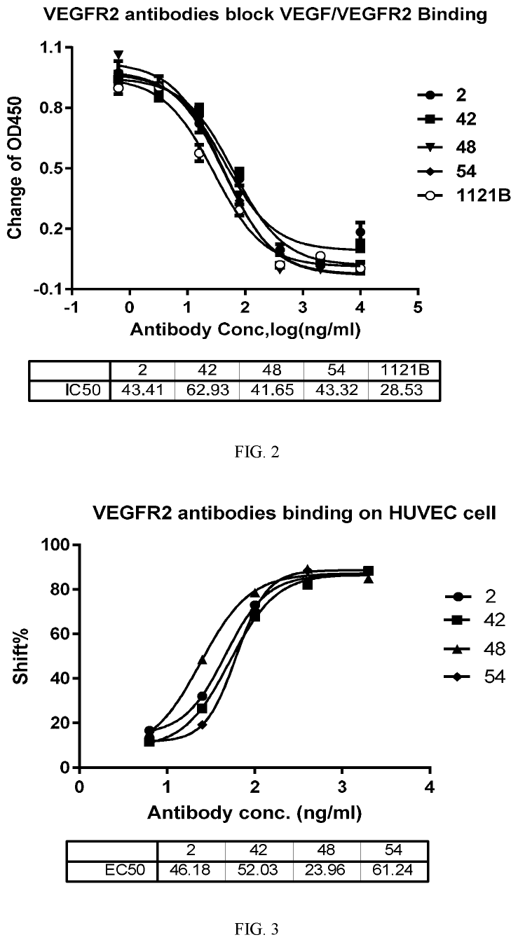

As shown in FIG. 2, 002, 048, 054 and 1121B had similar blockade activities with comparable IC50, indicating the former 3 antibodies may also inhibit activity of VEGF-A via blocking VEGF-A/VEGFR2 interaction, like 1121B.

### Example 6: Dose-Dependent Response of Binding of Purified VEGFR2 Antibodies to HUVEC Measured by FACS

Log phase HUVEC cells were collected, counted and resuspended in FACS buffer (5% BSA+PBS). 2×10∧5 cells were added to each tube and then washed once with PBS (1500 rpm, 5 mins, RT). Then 100p1/tube serial diluted VEGFR2 antibodies purified from hybridoma supernatant in FACS buffer were added into corresponding tubes and were incubated at 4° C. for 1 hr. The cells were then washed twice with 1 ml PBS, followed by adding 100 μl/tube of 2^{nd} antibody (1:400 anti-mIgG (H+L)-PE, Cell signaling #8887) in FACS buffer. The cells were incubated at 4° C. for 0.5 hr and then washed twice with PBS, followed by resuspending cells in 600 μl PBS for each sample. The cells were then transferred into FACS tube and the bindings of the antibodies to the cells were detected using flow cytometry (BD Accuri C6) (see FIG. 3).

The percentage of positive binding cells was calculated and fit by Graphpad Prism Software. As shown in FIG. 3, these hybridoma antibodies could bind to HUVEC cells, which is well-known of VEGFR2 expression on cell surface.

### Example 7: Gene Cloning and Sequencing of Hybridoma Antibodies

The sequences of the mouse anti-human VEGFR2 antibody light chain and heavy chain variable regions were obtained by the polymerase chain reaction (PCR) amplification technique known as 5′ RACE (rapid amplification of cDNA ends). Total RNA from 11B8 (002)/21B4(003)/5G4(048)/10D11(054) antibody producing hybridoma cell was isolated using Trizol (Invitrogen) and cDNA was synthesized using Superscript first strand synthesis system (Invitrogen) with Oligo (DT) 12-18 primer (Invitrogen). The variable regions of mouse IgG gene were cloned by PCR with MuIgG VH3′-2 and MuIg VH5′ leader primers for heavy chain variable region and MuIgK VL3′-1 and MuIgK VL5′ leader primer for light chain variable region (NOVAGEN). The resulting band for each antibody was cloned into pMD®18-T cloning vector and DNAs from 20 clones were submitted for sequencing and determined using ABI DNA sequencing instruments (Perkin Elmer). Consensus sequences were determined using Vector NTI Advance 10 software (Invitrogen).

Generation of chimeric antibodies: After sequencing analysis and confirmation, the variable region of the each above mentioned genes were cloned into a recombinant expression vector pCP-Hck/Hcgl. Briefly, pCP-Hck/Hcgl vector was digested by two steps of restriction enzyme first. Then the genes of the light chain variable region (VL) and heavy chain variable region (VH) were homologous recombined with digested vector. After transformation, clony PCR and sequence confirmation, the expression vectors of chimeric antibodies were generated.

### Example 8: Recombinant Chimeric Antibody Expression and Purification

The expression and purification of the recombinant chimeric antibody protein produced above were conducted by following methods: HEK293E cells cultured in Freestyle 293 Expression Medium with 10% of Pluronic F-68 at 1×10⁶ cell/ml were transfected with DNA vector with final concentration of 0.5 μg/ml and PEI (Polyethylenimine-linear, Polyscience) of 1.0 μg/ml. DNA to PEI ratio was 1:2. DNA and PEI complexes formed period with Optimal MEM should be 15 minutes at the room temperature. Transfected cells were cultured in the flasks with 5% CO₂, at 37° C. and at 125 rpm shaking speed. 1% Peptone medium was added at 22 to 26 hours post transfection.

Conditioned medium was harvested on day 6 and supernatant was centrifuged at 3,000 rpm for 30 minutes. The clarified conditioned medium was then loaded onto a pre-equilibrated 0.5 ml Protein A column, washed with 5 column volumes of 1XPBS and finally the bound IgG was eluted with 3 mL pH3.0 0.1M Glycine-HCL buffer. The eluted antibody protein was dialyzed to PBS and stored at −80° C. To remove endotoxin, the purified protein was further processed by passing through Hitrap DEAE Sepharose F.F. column and the resulting antibody was analyzed to determine the level of purity using size exclusion chromatography (Superdex 200 5/150 GL, G.E. Healthcare).

### Example 9: Binding of Purified Anti-VEGFR2 Chimeric Antibodies to Human and Rhesus VEGFR2 Protein

Same protocol of Example 4 except using HRP conjugated goat anti-human Fc antibody as secondary antibody. Binding to hVEGFR2-his and rhesus VEGFR2-his (Acrobiosystems, Cat #VE2-C52H3) were also analyzed head-to-head.

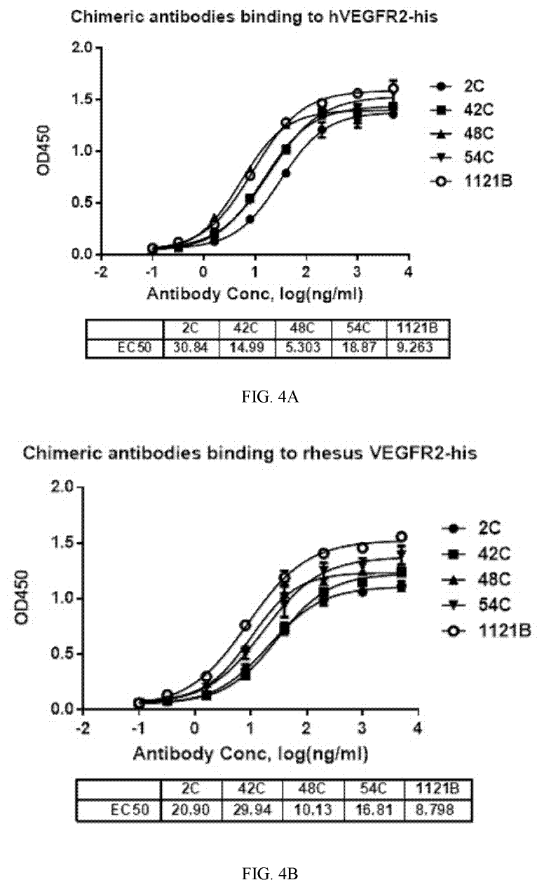

As shown in FIGS. 4A and 4B, 4 chimeric antibodies could bind to human and rhesus VEGFR2 with similar affinity, like 1121B, indicating the possibility of evaluating safety issue in rhesus monkey for further study.

### Example 10: Characterization of the Ability of Chimeric Anti-VEGFR2 Antibodies to Inhibit the Binding VEGF-A to hVEGFR2

For this assay, 0.25 μg/ml VEGF-A was coated on ELISA plate before a mixture of chimeric antibodies and VEGFR2 were added. To detect free VEGF-A (not bind to VEGFR2), 1:1000 diluted mouse serum (mouse previous immunized with human VEGFR2) were added and HRP conjugated goat anti-mouse Fc antibody was used as secondary antibody.

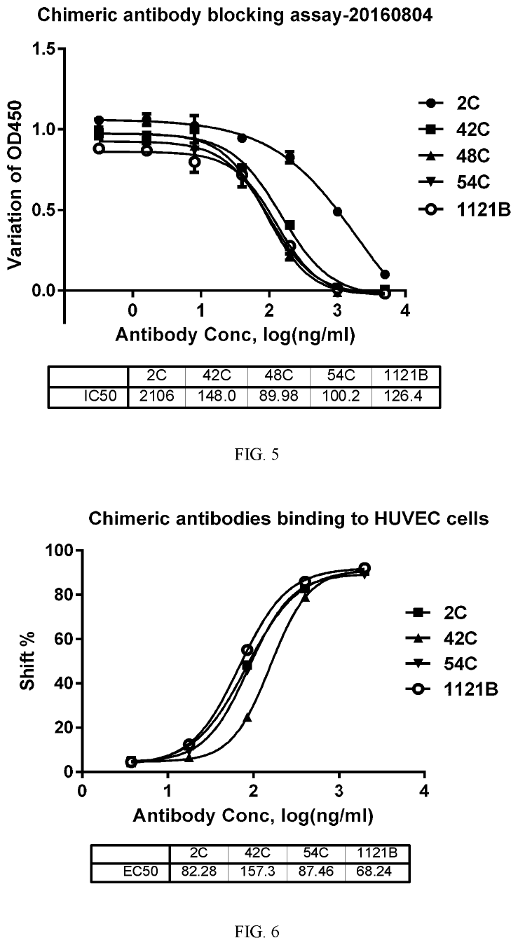

As shown in FIG. 5, 3 antibodies (042C, 048C and 054C) could inhibit VEGF-A binding to VEGFR2 as good as 1121B or even better. 002C showed much weaker neutralizing activity.

### Example 11: FACS Based Analysis of Binding of Select Purified Chimeric Antibodies to HUVEC

Same protocol of Example 6 except using 1:400 rabbit anti-hIgG (H+L)-PE, (Cell signaling #8887) as secondary antibody. As shown in FIG. 6, 002C and 054C bound to HUVEC cells with almost same EC50 of 1121B. Therefore, these VEGFR2 antibodies could function by binding to human VEGFR2 expressing on cell surface.

### Examples 12: Epitope Binning by Competition Assay

Similar protocol of ELISA binding assay described above. Briefly, 0.5 μg/ml hVEGFR2-his was coated onto the plate. 20 μg/ml competitor antibodies and 1.25 μg/ml biotinylated other antibodies were added simultaneously. After incubation of 3 hours, neutravidin conjugated HRP were added for detecting bound biotinylated antibody. The signal of bound biotinylated antibody will decrease if competitor antibody compete same epitope but not effect if different epitope.

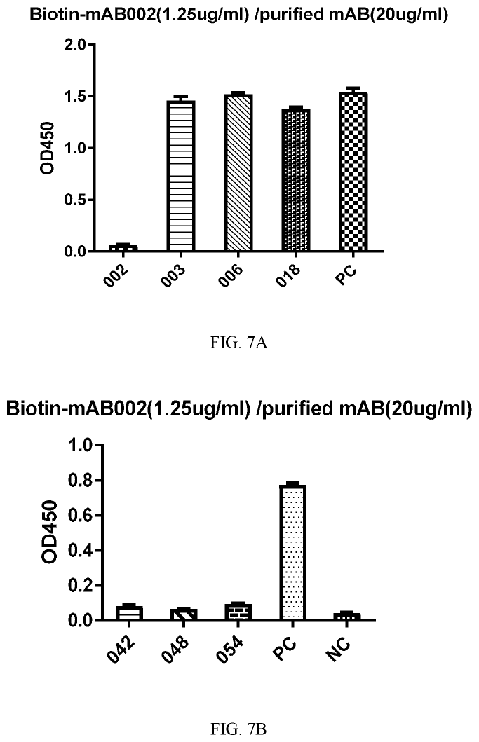

As shown in FIG. 7, Biotin-mAb002 was competed by 002, 042, 048 and 054, indicating that they may belong to same epitope group. And this group was later found with best blocking activity and same epitope of 1121B (data not shown).

Then diluted competitor hybridoma antibodies (002, 042, 048 and 054) and lower concentration (100 ng/ml) of chimeric antibodies (002C, 042C, 054C) were added for replacement following same protocol above. Anti-hIgG Fc-HRP was used as detection antibody.

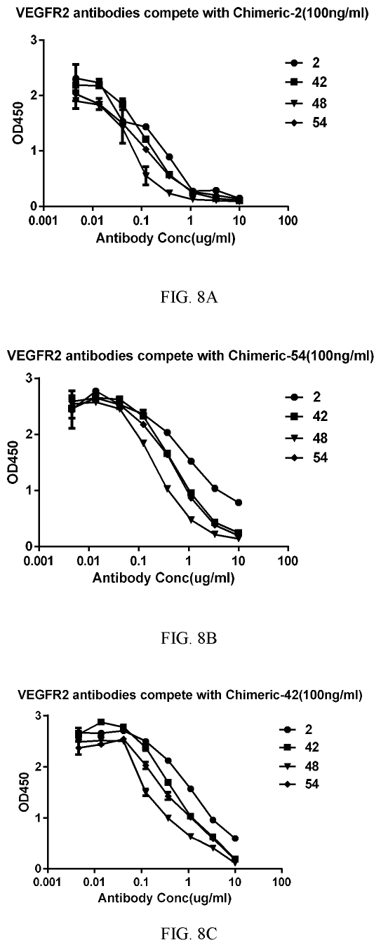

As shown in FIGS. 8A-8C, with increasing concentration of hybridoma antibodies, the chimeric one could not bind well or at all. Consistent with FIGS. 7A and 7B, hybridoma 002, 042, 048 and 054 could compete with chimeric ones, probably due to same epitope.

### Example 13: Profiling the Activities of Purified Chimeric Antibodies in Blocking VEGF-A Induced pVEGFR2 in HUVEC Cells

Cells expressing VEGFR2, such as HUVEC cell, will up regulate the expressing level of VEGFR2 upon VEGF-A stimulation. Then increased VEGFR2 could bind to VEGF-A and become dimerized form. The dimerization will induce Tyr phosphorylation of VEGFR2 following by activation of down stream pathway, such as MAPK/ERK and PI3K.

Briefly, HUVEC cells was seed into 12-well plates at a density of 1.5×10⁵ cells/well in culture medium with 10% FBS and incubated for 16 hours at 37° C., 5% CO₂ incubator, then the cells were starved for 4 hours with serum free medium. Different chimeric antibodies were added to each well incubated for 30 min, followed by adding 20 or 40 ng/ml VEGF-A incubated for 15 min. Finally, cellular lysate were prepared by adding 100 μl RIPA buffer (Thermo Scientific™,Cat #89900) containing complete protease inhibitor (Roche #04693132001) and phosphatase inhibitor (Pierce #1862495), incubating on ice for 15 minutes and centrifuged to collect supernatant. Equal amounts of the cellular lysates were resolved by 8% SDS-PAGE. The proteins were transferred to a PVDF membrane (Millipore) at a tank containing ice, run 150 min under 400 amp. The membrane was then incubated with a solution containing 5% BSA+TBST for 2 hours at RT. Then the membrane was incubated with a solution containing 1% BSA+TBST and rabbit anti-phospho-VEGFR2 (Tyr1175) mAB(CST, Cat #3770)/mouse anti-β-actin antibody (Abbkine, Cat #A0101502) overnight at 4° C.; The membranes were then washed 3x with TBST for 10 min each time with gentle rotation; The membrane was then incubated for 2 hours with HRP conjugated goat anti-rabbit IgG Fc pAB(Abcam, Cat #Ab97080)/RP conjugated goat anti-mouse IgG pAb(Abcam, Cat #ab97040) 1:1000 diluted in 1% BSA+TBST. The membranes were then washed 3x with TBST for 10 min for each time with gentle rotation; Finally, 1 ml ECL mixture (Pierce) was added onto PVDF membrance for signal exposure.

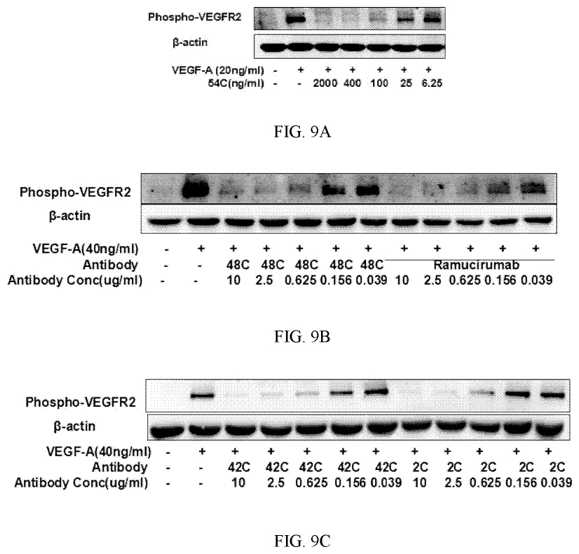

As shown in FIGS. 9A, 9B and 9C, both 20 and 40 ng/ml of VEGF-A could quickly induce VEGFR2 phosphorylation. And 054C, 048C, 042C and 002C could reverse this stimulation with dose-dependent manner as well as Ramucirumab.

### Example 14: Generation and Characterization of Humanized Antibodies

### 1. Generation, Expression and Purification of Humanized Antibodies

The sequences of the variable domains of mouse antibodies 054 and 048 were used to identify the germline sequence with the highest homology to the murine framework. Computer-modelling was used to design humanized variants with CDR grafting and back mutation.

### Ab-54

Human germline framework sequence VK/2D-40 for light chain and VH/3-21 for heavy chain were used for CDR grafting, respectively.

Germline sequence for Mab54 HC:

VH/3-21 (Mab54-germline, SEQ ID NO: 92):
EVQLVESGGGLVKPGGSLRLSCAASGFTFSSYSMNWVRQAPGKGLEWVSSISSSSSYIYYADSVKGRFTISRD
NAKNSLYLQMNSLRAEDTAVYYCARELDGNYDYWGQGTTLTVSS
VH/3-21 variant 1 (Mab54-Hzd-HC-V1, SEQ ID NO: 93):
EVQLVESGGGLVKPGGSLKLSCAASGFTFSMYGMSWVRQTPGKRLEWVASISIGGSYTYYADSVKGRFTISRD
NAKNTLYLQMNSLKAEDTAVYYCARELDGNYDYWGQGTTLTVSS
VH/3-21 variant 2 (Mab54-HC-V2, SEQ ID NO: 94):
EVQLVESGGGLVKPGGSLRLSCAASGFTFSMYGMSWVRQAPGKRLEWVASISIGGSYTYYADSVKGRFTISRD
NAKNTLYLQMNSLRAEDTAVYYCARELDGNYDYWGQGTTLTVSS
Germline sequence for Mab54 LC:
VK/2D-40 (Mab54 LC germline, SEQ ID NO: 95):
DIVMTQTPLSLPVTPGEPASISCRSSQSLLDSDDGNTYLDWYLQKPGQSPQLLIYTLSYRASGVPDRESGSGS
GTDFTLKISRVEAEDVGVYYCMQRIEFP
VK/2D-40 variant 3 (Mab54-Hzd-LC-V1, SEQ ID NO: 96):
DIVITQDELSLPVTFGESVSISCRSSKSLLYKDGKTYLNWFLQRPGQSPQLLIYLMSTRASGVSDRESGSGSG
TDFTLKISRVEAEDVGIYYCQQLVEYPFTEGSGTKLEIK
VK/2D-40 variant 4 (Mab54-Hzd-LC-V2, SEQ ID NO: 97):
DIVITQTPLSLPVTPGESVSISCRSSKSLLYKDGKTYLNWFLQRPGQSPQLLIYLMSTRASGVPDRESGSGSG
TDFTLKISRVEAEDVGVYYCQQLVEYPFTFGSGTKLEIK

### Ab-48

Human germ line framework sequence VK/2D-40 for light chain and VH/3-21 for heavy chain were used for CDR grafting, respectively.

Germline sequence for Mab48 HC:

VH/3-21 (Mab48-HC germline, SEQ ID NO: 102):
EVQLVESGGGLVKPGGSLRLSCAASGFTFSSYSMNWVRQAPGKGLEWVSSISSSSSYIYYADSVKGRFTISRD
NAKNSLYLQMNSLRAEDTAVYYCAR
VH/3-21 variant 5 (Mab48-Hzd-HC-V3, SEQ ID NO: 98):
EVQLVESGGGLVKPGGSLRLSCAASGFTFSMYGMSWVRQAPGKRLEWVASISIGGSYTYYADSVKGRFTISRD
NAKNTLYLQMNSLRAEDTAVYYCAREMDGNYDYWGHGTTLTVSS

Germline and light chain sequence for Mab48 were same as Mab54.

Therefore, there were 4 variants of humanized Mab54, labeled as 54-H1L1, 54-H2L1, 54-H1L2, 54-H2L2 for short, and 2 variants of humanized Mab48, labeled as 48-H3L1, 48-H3L2 for short. In fact, VH/3-21 variant 5 is almost same with VH/3-21 variant 2, except one amino acid in HCDR3. That is also consistent with the similar biological activities of two antibodies shown in this patent.

The above heavy chain and light chain cDNAs were synthesized and fused with the constant region of human IgG1 in the Fc region (The numbering of the heavy chain residues described herein are according to the EU index of Kabat (see Kabat et al., “Proteins of Immunological Interest”, US Dept. of Health & Human Services (1983))). The variable regions of the heavy chain and light chain of the selected antibody genes were synthesized and cloned into an expression vector and the large scale DNA was prepared using PureYield™ Plasmid Maxiprep System from Promega. Transfection was carried out using the ExpiFectamine™ 293 Reagent from Invitrogen according to the manufacturer's protocol. Supernatants were harvested when the cell viability was around 50%. Protein A beads and clean supernatants were incubated at 4° C. for 2 hr with rocking before going through a column. The Protein A beads inside the column were washed with PBS, and 100 mM Glycine buffer (pH3.0) was used to elute the antibody, which was dialyzed against the PBS buffer (137 mM NaCl, 2.7 mM KCl 10 mM Na₂HPO₄, 2 mM KH₂PO₄, pH7.4) overnight at 4° C. Finally, endotoxin was removed using Pierce High Capacity Endotoxin Removal Resin (Invitrogen, Catalog number: 88271). The purified antibody was characterized by SDS-PAGE and SEC-HPLC.

2. Binding of Humanized Antibodies to hVEGFR2 and Rhesus VEGFR2 in ELISA

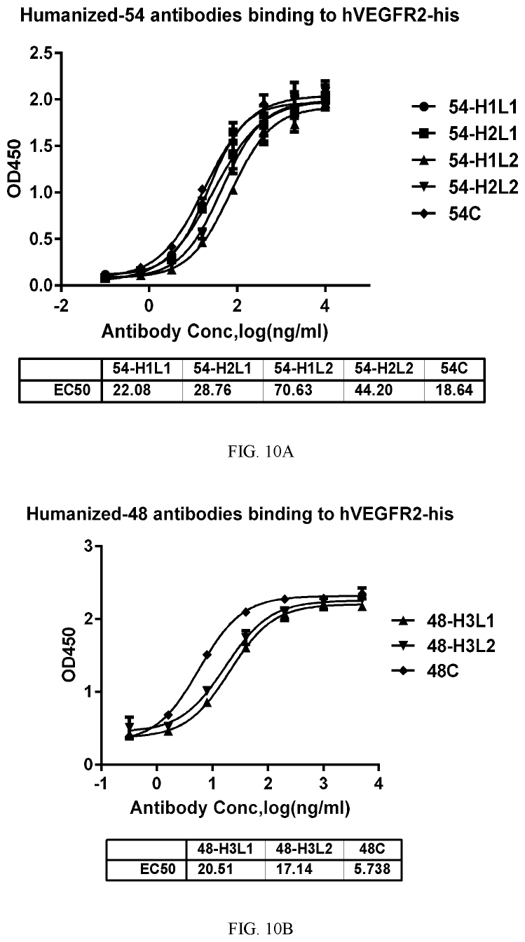

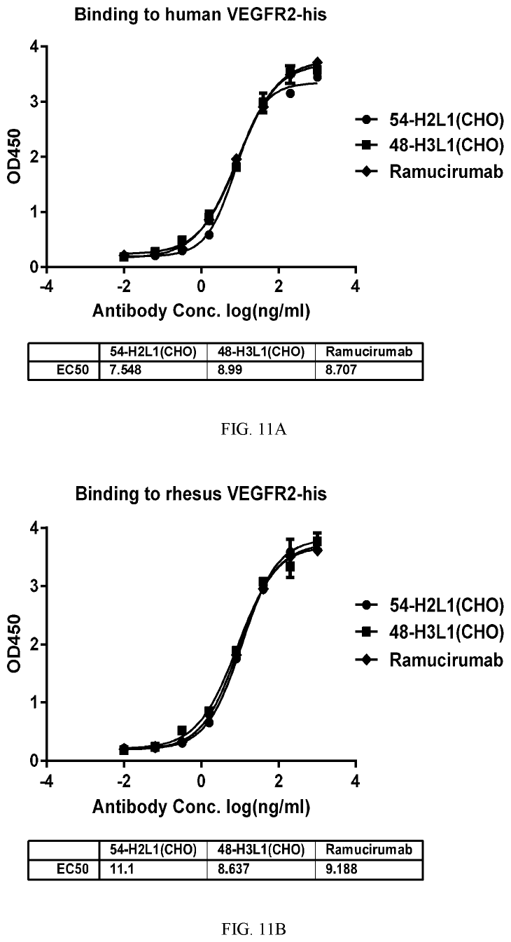

Same protocol of Example 9. Humanized 054 and 048 antibodies expressed by 293T cells were tested first (see FIGS. 10A and 10B).

Then two humanized antibodies 54-H2L1 and 48-H3L1 were expressed by CHO cells for further estimation, which labeled as 54-H2L1 (CHO) and 48-H3L1 (CHO). Cross-species binding to rhesus VEGFR2-his is maintained, which is a very important feature for therapeutic antibody development (see FIGS. 11A and 11B).

### 3. Specificity of Binding of Humanized VEGFR2 Antibodies

Briefly, 1 μg/ml human VEGFR1 (Sino Biological, cat #10136-H08H), VEGFR2 (Acrobiosystem, cat #KDR-H5227) and VEGFR3 (Sino Biological, cat #10806-H08H) protein were coated overnight at 4° C. 1 μg/ml humanized antibodies (48-H3L1 and 54-H2L1, produced in CHO cells or Ramucirumab from Eli Lilly Germany, lot #20150819) were added for binding to coated antigen. Secondary antibody HRP conjugated goat anti-human Fc antibody were added for detection of binding signal.

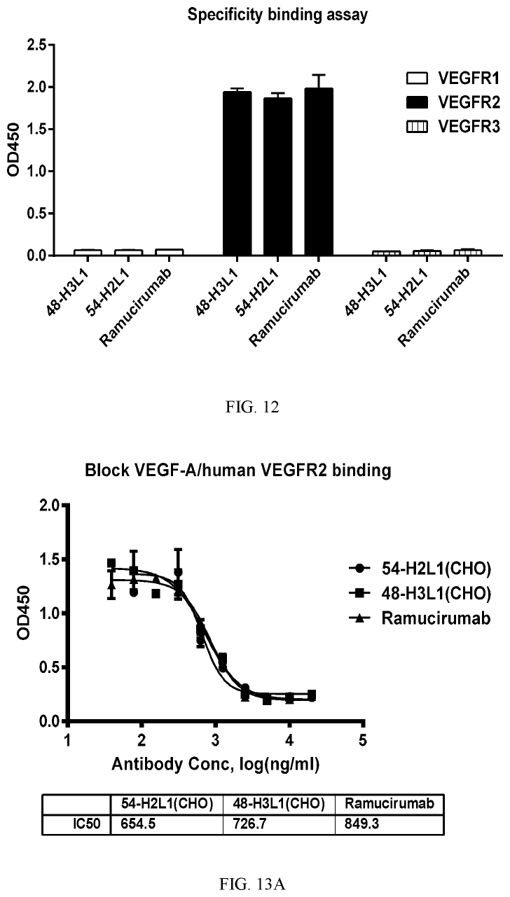

As shown in FIG. 12, VEGFR2 antibodies 48-H3L1, 54-H2L1 and Ramucirumab only bind to VEGFR2 but not VEGFR1 or VEGFR3, indicating the high specificity of these antibodies.

4. Blocking of hVEGF-A Binding to hVEGFR2 and Rhesus VEGFR2 in ELISA

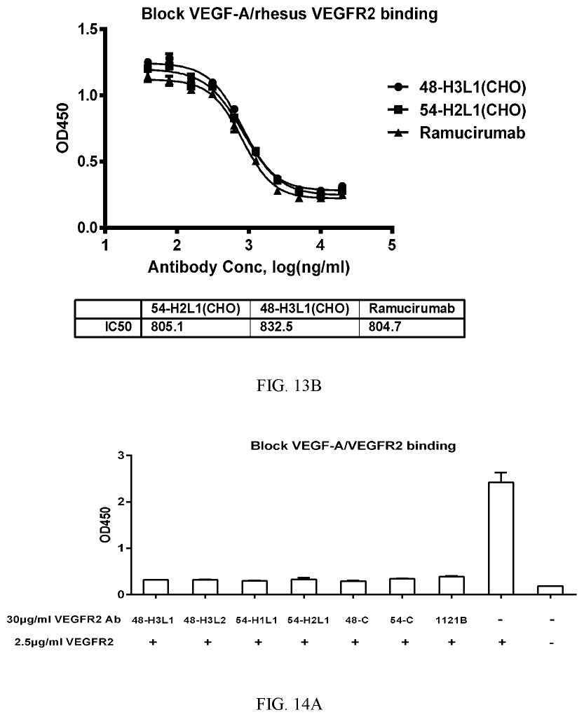

Same protocol of Example 10. Briefly, 0.25 μg/ml VEGF-A was coated overnight at 4° C. After blocking, serial diluted 54-H2L1, 48-H3L1, Ramucirumab and 1 μg/ml human VEGFR2 or rhesus VEGFR2 were added at the same time for 2 hour-incubation. After wash, mouse serum containing polyclone VEGFR2 antibody and HRP conjugated goat anti-mouse Fc antibody were added for detection of free VEGF-A coated on the plates (see FIGS. 13A and 13B).

As shown in FIGS. 13A and 13B, two humanized antibodies could block VEGF-A interacting with either human VEGFR2 or rhesus VEGFR2. The neutralizing activities of 48-H3L1 and 54-H2L1 were similar to that of Ramucirumab or may even be better.

### 5. Blocking of VEGF-C and VEGF-D Binding to VEGFR2 by Humanized Antibodies

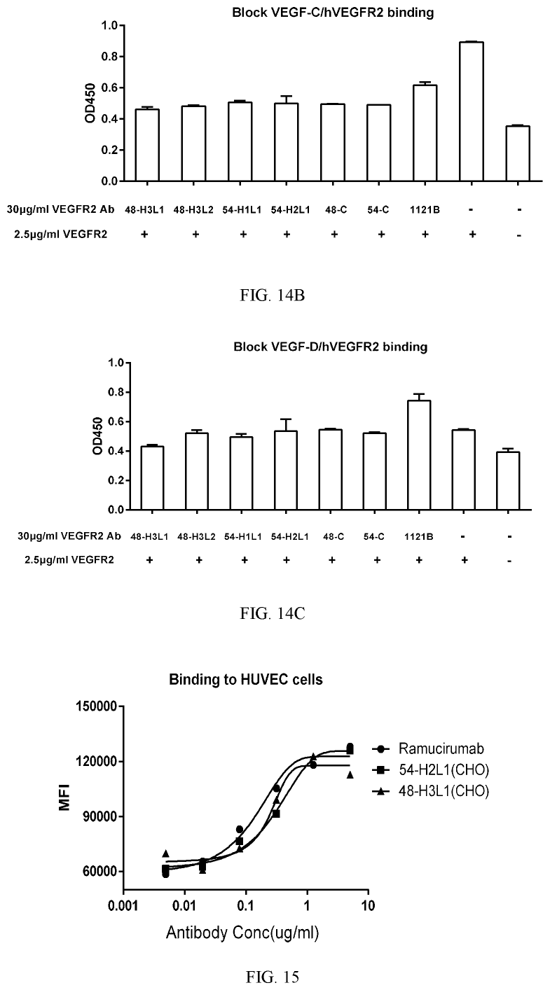

Similar protocol of Example 10. Briefly, 0.5 μg/ml VEGF-A or VEGF-C or VEGF-D was coated and 30 μg/ml VEGFR2 antibodies and 2.5 μg/ml human VEGFR2 were added at the same time for 2 hour-incubation. Using same detection method of Example 10, free VEGF-A or VEGF-C or VEGF-D coated on the plates could be measured (see FIGS. 14A, 14B and 14C).

As shown in FIGS. 14A, 14B and 14C, VEGFR2 antibodies, including 1121B, completely blocked VEGF-A binding to VEGFR2 but not VEGF-C or VEGF-D. Although a little decrease of VEGF-C binding was seen, the window was too limited to draw a conclusion.

6. Bindings of Humanized Antibodies to hVEGFR2 on HUVEC by FACS

Same methods with Example 11. Briefly, Log phase HUVEC cells were collected and added to each tube. Then 100 μl/tube serial diluted humanized antibodies in FACS buffer were added and kept at 4° C. for 1 hr. Binding of antibodies to HUVEC was detected by PE signal of the 2^{nd} antibody (anti-hIgG (H+L)-PE), which was added after the 1^{st} antibodies. (see FIG. 15).

7. Blocking of hVEGF-A Induced hVEGFR2 Phosphorylation in HUVEC

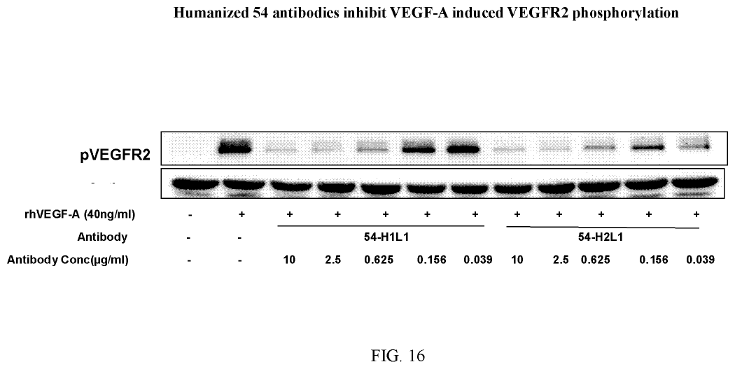

Same methods of Example 13. Briefly, HUVEC cells was seed into 12-well plates in culture medium with 10% FBS and incubated for 16 hours, then starved for 4 hours without serum. Diluted humanized antibodies were added to each well incubated for 30 min, followed by adding 40 ng/ml VEGF-A incubated for 15 min. Finally, the cellular lysate were prepared and detected phospho-VEGFR2 and j-actin by Western Blot described in Example 13. (see FIG. 16).

As shown in FIG. 16, two variants of humanized 54 could decrease the amount of phosphorylated VEGFR2 induced by VEGF-A. The inhibition effect of 54-H2L1 was more potent than 54-H1L1.

### Example 15: Evaluation of the Ability of the Purified Humanized Antibodies to Inhibit HUVEC Proliferation and Tube Formation

Log-phased HUVEC cells were seed into 96-well plates at a density of 5×10³ cells/well in culture medium with 10% FBS and incubated overnight at 37° C., 5% CO₂ incubator, then the cells were starved for 4 hours with 50 μl/well of serum free medium. Serial diluted humanized antibodies were added 50 μl/well and incubated for 30 min, followed by adding 50p/well VEGF-A (final concentration:20 ng/ml) and incubated for 72 hours. At the end of cell culture, 20p1/well of Cell Titer-Glo® Luminescent kit was added and the supernatant was transferred to a new white plate for luminescent signal reading.

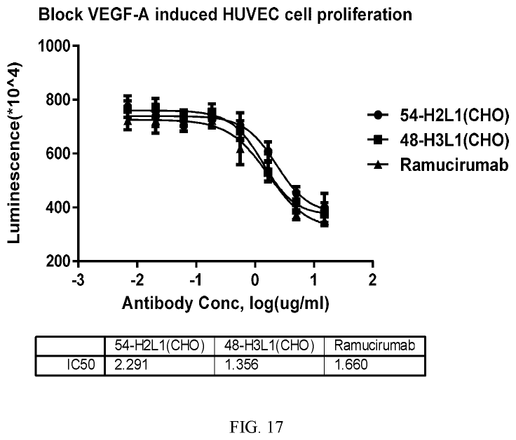

HUVEC cells stimulated by VEGF family will be activated and proliferate. Blockade of VEGFR2 and VEGF could reverse the stimulation effect. As shown in FIG. 17, humanized 54 and 48 antibodies could inhibit proliferation as well as Ramucirumab with dose-dependent manner, indicating that they have blocked VEGF/VEGFR2 signal pathway of HUVEC cells.

### Example 16: Evaluation of the Binding Affinity of Select Mouse Hybridoma or Humanized Anti-VEGFR2 Antibodies Via Biacore

A CM5 sensor chip was activated in each flow cell by 7-min injection (10 μl/min) of freshly prepared 1:1 50 mM N-hydroxysuccinimide (NHS): 200 mM 1-ethyl-3-(3-dimethylaminopropyl)-carbodiimide (EDC). Then anti-human Fc antibody (GE Healthcare) in a concentration of 10 μg/ml in 10 mM sodium acetate buffer PH 5.0 was injected onto the activated chip at 10 μl/min (HBS-EP running buffer: 10 mM HEPES, 150 mM NaCl, 3.4 mM EDTA, 0.005% surfactant P20, pH 7.4). The remaining active coupling sites were blocked by 7 min injection of 1M ethanolamine at 10 μl/min. The immobilization level of each flow cell is ˜9000RU. Antibodies were captured in FC2 by anti-human Fc IgG or anti-mouse Fc (GE Healthcare) to 200˜300RU. FC1 was used as the reference cell. Antigen was injected at varying concentrations (2.5 nM, 5 nM, 10 nM, 20 nM, 40 nM and 80 nM) after capture of antibody. The association time for antibody binding antigen is 180s. Surface Regeneration condition is 120s at 10 μl/min in Gly pH1.5. The signals with captured antibody subtracted from those without captured antibody were calculated with Biacore X100 evaluation software ver. 2.0 (Biacore).

TABLE 7
Affinity parameters of antibodies binding to antigen

| Antibody | clones | ka (1/Ms) | kd (1/s) | KD (M) |
|---|---|---|---|---|
| mAb002 | 11B8C2C11 | 5.62E+04 | 3.36E−04 | 5.98E−09 |
| mAb003 | 21B4G9B4 | 1.26E+05 | 2.95E−05 | 2.35E−10 |
| mAb006 | 27E8A9D5 | 1.27E+05 | 1.86E−04 | 1.47E−09 |
| mAb018 | 16D9G2E8 | 8.84E+04 | 4.52E−04 | 5.11E−09 |
| mAb042 | 8G11G1B2C6 | 8.51E+04 | 4.88E−05 | 5.73E−10 |
| mAb054 | 10D11F4D1F6 | 9.18E+04 | 9.12E−05 | 9.93E−10 |
| mAb048 | 5G4H2C3D3 | 1.14E+06 | <1.0E−7 | <1.0E−12 |
| mAb054 | 10D11F4D1F6 | 9.70E+04 | 1.22E+06 | 1.26E−10 |
| mAb054 | 54-H1L1 | 4.51E+04 | 5.15E−05 | 1.14E−09 |
| mAb054 | 54-H1L2 | 3.77E+04 | 1.39E−04 | 3.68E−09 |
| mAb054 | 54-H2L1 | 5.48E+04 | 1.38E−04 | 2.51E−09 |
| mAb054 | 54-H2L2 | 2.97E+04 | 1.28E−04 | 4.30E−09 |
| mAb054 | 54-C | 4.60E+04 | 1.13E−04 | 2.45E−09 |

It is known that the interaction between human VEGF-A and VEGFR2-Fc has a K_{D} of about 89 pM (doi: 10.1007/s10456-011-9249-6).

### Example 17: Epitope Mapping of Select Anti-VEGFR2 Via Alanine Scan

### 1. Generation of Mutant Human VEGFR2 Recombinant Proteins

The cDNA coding for extracellular human VEGFR2 (amino acid 1-1356) and Fc fragment of human IgG1 was synthesized in vitro (SEQ ID NO: 103 is the amino acid sequence). Variants of human VEGFR2 with single amino acid change at designated position as listed below were amplified by overlapping PCR as described below and using primers (Dr. Oligo BLP-192, Biolytic) as shown in (Table 8). The resulting fragment was digested with restriction enzymes for Hind III and BamH I at 5′ and 3′ ends respectively. The PCR product was then cloned into the pcDNA3.1 (+) vector by method of homologous recombination using Syno assembly mix reagent (Synbio) following manufacturer's instructions. Plasmid was purified QIAGEN Plasmid Mega Kit (QIAGEN).

Amino acid sequence (SEQ ID NO: 103):
MQSKVLLAVALWLCVETRAASVGLPSVSLDLPRLSIQKDILTIKANTTLQITCRGQRDLDWLWPNNQ
SGSEQRVEVTECSDGLFCKTLTIPKVIGNDTGAYKCFYRETDLASVIYVYVQDYRSPFIASVSDQHG
VVYITENKNKTVVIPCLGSISNLNVSLCARYPEKRFVPDGNRISWDSKKGFTIPSYMISYAGMVFCE
AKINDESYQSIMYIVVVVGYRIYDVVLSPSHGIELSVGEKLVLNCTARTELNVGIDENWEYPSSKHQ
HKKLVNRDLKTQSGSEMKKELSTLTIDGVTRSDQGLYTCAASSGLMTKKNSTFVRVHEKPFVAFGSG
MESLVEATVGERVRIPAKYLGYPPPEIKWYKNGIPLESNHTIKAGHVLTIMEVSERDTGNYTVILTN
PISKEKQSHVVSLVVYVPPQIGEKSLISPVDSYQYGTTQTLTCTVYAIPPPHHIHWYWQLEEECANE
PSQAVSVINPYPCEEWRSVEDFQGGNKIEVNKNQFALIEGKNKTVSTLVIQAANVSALYKCEAVNKV
GRGERVISFHVTRGPEITLQPDMQPTEQESVSLWCTADRSTFENLTWYKLGPQPLPIHVGELPTPVC
KNLDTLWKLNATMFSNSINDILIMELKNASLQDQGDYVCLAQDRKTKKRHCVVRQLTVLERVAPTIT
GNLENQTTSIGESIEVSCTASGNPPPQIMWFKDNETLVEDSE

Using wild-type VEGFR2 plasmid generated above (SEQ ID NOs: 103 and 104) as template, two segments of an integrated sequence were generated with mega primers (Table 8), and ligation was accomplished by homologous recombination. The product was then cloned into the pcDNA3.1 (+) vector, after screening the individual positive colony through sequencing to identify the variants, VEGFR2 mutants had been proved to be generated successfully. PCR procedure and conditions as follows:

TABLE 8
Variants mutant primers sequence
Step 1: to generate two mega fragments of variants

| | μl |
|---|---|
| ddH2O | 35 |
| 5 × S15 PCR buffer | 10 |
| 10 mM dNTP | 1 |
| F primer | 1 |
| R primer | 1 |
| PCR product | 1 |
| S15 polymerase | 1 |
| | 50 |

Initial denaturation: 98° C. 1 min
denaturation: 98° C. 15 s
Annealing: 58° C. 30 s
Extension: 72° C. 30 s per Kb
Eventually extension: 72° C. 3 mins 30 cycles
Step 2: to join the two pieces together
Set up the following reaction on ice
(homologous recombination):

| | |
|---|---|
| Syno assembly mix | 10 μl |
| Sequence products 1 | 2 μl |
| Sequence products 2 | 2 μl |
| Deionized H2O | 6 μl |
| Total Volume | 20 μl |

Other 20 variants are constructed by another company, Genewiz (Suzhou, China). Similar methods with Synbio Tech are performed to synthesize VEGFR2 variants.

Subsequently, these plasmids of mutants and wild-type VEGFR2 were transfected into 293T (ATCC® CRL3216) cell line. First, Seeding 5×10⁶ 293T cells into 60 mm dish, make sure primary ratio is at 60%˜80% for transfection. Then dilute 10 g DNA in 400 μl 1×HBS, incubate for about 5 min. Add 10 125 kDa linear PEI transfection reagent (dissolved in 1×HBS, 1 mg/ml stock solution) to the above mixture, make sure DNA/PEI ratio is 1:2.5. Then the mixture was added into 293T dish drop by drop. Change the medium and replace with complete DMEM about 6-8 hrs later. After 72 hrs, cell culture supernatant was collected with 0.22 m filter respectively, then stored at −80° C. for use.

2. Binding of VEGFR2 Antibodies to hVEGFR2 Mutants by ELISA

Supernatant was used to detect the binding of VEGFR2 antibodies by ELISA as described below.

### 2.1 for Mouse Abs:

0.5 μg/ml anti-human Fe antibody (Abcam) was coated for one hour at room temperature. 160 μl DMEM supernatant containing various mutant human VEGFR2-Fc proteins or 500 ng/ml wild type human VEGFR2-Fc protein in DMEM were added to each well and incubated for 1 hour at room temperature. Mouse anti-VEGFR2 antibodies at 0.5 μg/ml were added and incubated for 1 hour at RT. Then HRP conjugated goat anti-mouse IgG Fe antibody and TMB were added sequentially and the reaction was terminated using 0.64M H₂SO₄. The plates were read on a Thermo Multiscan FC at 450 nM.

2.2 for Abs with Human Fc (Chimeric or Humanized Antibodies):

Similar protocol of 2.1 except adding chimeric or humanized antibodies for binding to mutant VEGFR2-Fc and HRP conjugated anti-hIgG Fe as secondary antibody.

Table 9 summarizes the key residues on hVEGFR2 that are required for each individual antibody tested to human VEGFR2 in the ELISA assay. The amino acid residue mutations on the human VEGFR2 proteins that lead to significantly reduced binding signal relative to that of the wild type protein is marked.

TABLE 9
Summary of the key residues on hVEGFR2 for each antibody

| Code | Position | Mutant | 02 | 02C | 42 | 42C | 48 | 48C | 54 | 54C | 1121b |
|---|---|---|---|---|---|---|---|---|---|---|---|
| 1 | | KDR | | | | | | | | | |
| | | WT | | | | | | | | | |
| 8 | 133 | H133A | | | | | | | | | |
| 21 | 135 | V135A | | | | | | | | | |
| 9 | 137 | Y137A | | ★★ | | | | | | | |
| 22 | 164 | R164A | | | ★ | | | | ★ | | |
| 10 | 165 | Y165A | ★ | | ★ | ★ | ★ | ★ | ★ | ★ | ★ |
| 23 | 197 | M197A | | | | | | | | | |
| 11 | 215 | I215A | | | | | | | | | |
| 24 | 217 | V217A | | | | | | | | | |
| 12 | 218 | V218A | | ★★ | | | | | | | |
| 2 | 221 | Y221A | | ★★★ | | ★★ | | ★★★ | | ★★ | |
| 15 | 222 | R222A | | ★ | | ★★★ | | ★★★ | | ★★★ | ★★ |
| 3 | 251 | E251A | | ★★★ | | ★★ | | ★★ | | ★★ | |
| 13 | 252 | L252A | | ★ | | | | | | | |
| 25 | 253 | N253A | | ★★★ | | | | ★ | | ★★ | |
| 16 | 255 | G255A | | ★★★ | | ★★ | | ★★ | | ★★ | ★ |
| 4 | 257 | D257A | | ★★ | ★★★ | ★★★ | | ★★★ | ★★ | ★★★ | ★★★ |
| 17 | 259 | N259A | | | | | | | | | |
| 5 | 261 | E261A | | | | | | | | | |
| 14 | 286 | K286A | | ★★ | | | | | | | |
| 18 | 312 | G312A | | | | ★★ | | ★★ | | ★★ | |
| 6 | 313 | L313A | ★★★ | ★★★ | | ★★ | | ★★★ | | ★★ | ★★ |
| 19 | 314 | M314A | | ★ | | ★★ | | ★★ | | ★★ | |
| 7 | 315 | T315A | | ★★★ | | ★★★ | | ★★★ | | ★★★ | ★★ |
| 20 | 316 | K316A | | ★ | | ★★ | | ★★ | | ★★ | |

### Example 18: Large Scale Production of Anti-VEGFR2 Antibodies for In Vivo Studies

Antibodies were produced using transient expression in CHO-K1 cells. The produced antibodies were purified using protein-A affinity column and upon desalting, the antibodies were formulated into PBS at 5 mg/ml with endotoxin level at <3 unit/mg. The resulting antibodies were characterized for purity using SDS-PAGE gel and SEC-HPLC.

### Transient Expression and Purification of Recombinant Antibody Using CHO-K1 Cells

The variable region of the heavy chain and light chain selected antibody genes were synthesized and cloned into an expression vector. The resulting expression contrast was used to transfect CHO-K1 cell adapted to grow in serum free medium. After 10 days' growth in 10 L Applikon bioreactor, the culture medium was harvested and the cells and cell debris were removed using ultra-filters. The clarified supernatant was then concentrated by ultrafiltration and uploaded onto a prepared Protein A (human IgG) or G-sepharose (mouse IgG) column. After being washed with equilibration buffer to baseline under monitoring by a UV monitor, the column was then eluted with 0.1 M citric acid, pH 3.5, and the eluted antibody was immediately neutralized with 1.0 M Tris-HCl buffer, pH 8.0, and dialyzed against PBS, pH 7.2 (Invitrogen), overnight at 2-8° C. with 2 buffer exchanges. The purified antibody was filtered through 0.22 μm sterile syringe filters and stored in aliquots at −80° C. or below.

### Example 19: In Vivo Evaluation of the Chimeric Anti-Human VEGFR2 Antibodies in HL-60 Tumor Model

In order to evaluate anti-tumor efficacy of VEGFR2 candidate antibodies, HL-60 mouse model was set up and many symptoms were estimated for efficacy. Briefly, 4-6 weeks female NOD/SCID mice were randomized grouped into 4 groups. After treatment of CTX (150mpk, i.p.), 1.5×10∧7 cells/mice were injected by i.v the next day. And 3 days after HL-60 cells injection, mice were treated with different antibodies listed in following table (Table 10).

TABLE 10
Grouping and schedule of treatment (n = 6)

| Group | Dosage | Schedule of treatment |
|---|---|---|
| Negative Control (saline) | — | twice/week × 4 |
| 1121B | 5 mpk | twice/week × 4 |
| 54-C | 5 mpk | twice/week × 4 |
| 2-C | 5 mpk | twice/week × 4 |

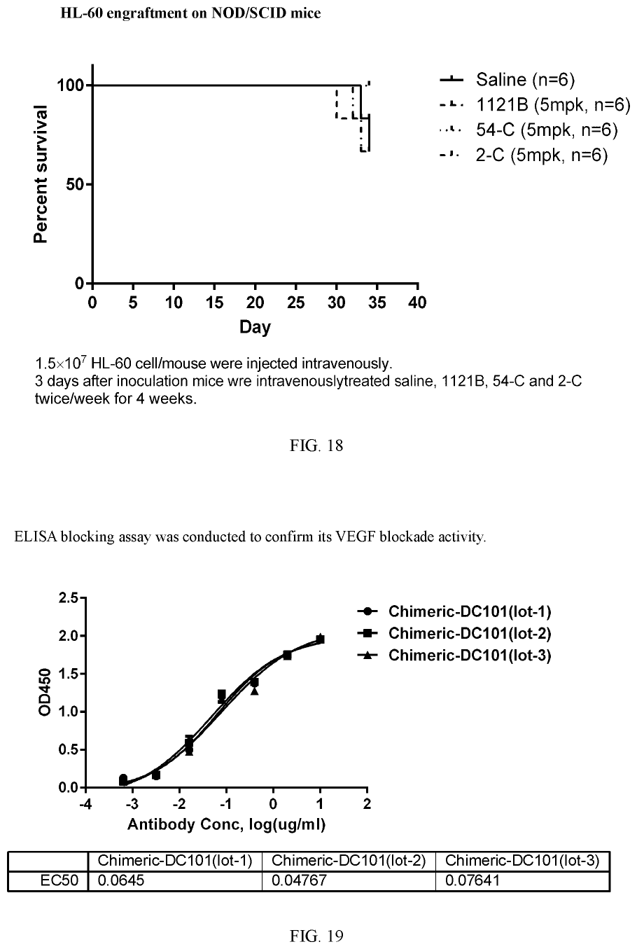

As shown in FIG. 18 and Table 11, 54-C had best survival percentage and condition, comparing with other groups. No animal died with 54-C treatment but 2 died in negative control group and 2-C treatment group. As we know, VEGFR2 activation occurs via binding to its ligand VEGF-A that mediates HL-60 cell proliferation through autocrine signaling. This may explain the tumor inhibition of VEGFR2 neutralizing antibody 54-C and 1121B.

TABLE 11
Symptoms and death of each treatment groups

| | Hindlimb | Hair | Swollen | | |
|---|---|---|---|---|---|
| Group | Weakness | Disheveled | Head | Thin | Death |
| Negative | 4/4 | 3/4 | 4/4 | 2/4 | 2/6 |
| Control | | | | | |
| 1121B | 4/5 | 4/5 | 4/5 | 0/6 | 1/6 |
| 54-C | 5/6 | 3/6 | 5/6 | 2/6 | 0/6 |
| 2-C | 3/4 | 3/4 | 3/4 | 0/4 | 2/6 |

### Example 20: Evaluation of the Anti-Mouse VEGFR2 Antibody DC101

### 1. In Vitro Evaluation of the Anti-Mouse VEGFR2 Antibody DC101

We recombined and expressed anti-mouse VEGFR2 antibody DC101 according to its sequence of Fab region listed in the Table 12 below. For Fc region of DC101, rat IgG1 Fc was replaced with human IgG1 Fc and named as chimeric-DC101. Then chimeric-DC101 was evaluated by in vitro assays to confirm its bioactivity.

TABLE 12
Variable chain sequences of DC101

| | VH | VL |
|---|---|---|
| DC101 | SEQ ID NO: 40 | SEQ ID NO: 99 |
| | MEFGLSWLFLVAILKGVQCEVQLVETGGGLVQPGNSLKLSCAT | MDMRVPAQLLGLLLLWFPGSRCDIVLTQSP |
| | SGFIFSTTWMNWIRQTPGKRLEWLAQIEDKSNNYFISYSESVKG | ALAVSLEQRATISCKTSQNVDYYGISYLHWYQ |
| | RFTISRDDSKSSVYLQMNNLKEEDTAIYYCSWKYRSNYYFDYWG | QKPGQQPKLLIYEGSNLASGIPARFSGSGSGT |
| | QGVMVTVSSASTKGPSVFPLAPSSKSTSGGTAALGCLVKDYFPE | DFTLTIDPVEADDIVTYYCQQSKDYPYTFGAG |
| | PVTVSWNSGALTSGVHTFPAVLQSSGLYSLSSVVTVPSSSLGTQT | TKLELKRTVAAPSVFIFPPSDEQLKSGTASVVC |
| | YICNVNHKPSNTKVDKKVEPKSCDKTHTCPPCPAPEAAGGPSVF | LLNNFYPREAKVQWKVDNALQSGNSQESVT |
| | LFPPKPKDTLMISRTPEVTCVVVDVSHEDPEVKFNWYVDGVEV | EQDSKDSTYSLSSTLTLSKADYEKHKVYACEVT |
| | HNAKTKPREEQYNSTYRVVSVLTVLHQDWLNGKEYKCKVSNKA | HQGLSSPVTKSFNRGEC |
| | LPAPIEKTISKAKGQPREPQVYTLPPSREEMTKNQVSLTCLVKGF | |
| | YPSDIAVEWESNGQPENNYKTTPPVLDSDGSFFLYSKLTVDKSR | |
| | WQQGNVFSCSVMHEALHNHYTQKSLSLSPGK | |

Sequences in bold in the Table 12 represent the signal peptide of the VH and VL respectively.

ELISA binding assay was conducted to confirm its binding affinity. Briefly, ELISA plates were coated with 500 ng/mL mouse VEGFR2 in coating buffer (0.16% Na₂CO₃, 0.30% NaHCO₃) and incubated at 4° C. overnight. After twice washing, blocking buffer (1% BSA, 1% goat serum, 0.05% Tween20 in PBS) was added and plates were incubated at room temperature for 2 hours. After three times washing, serial diluted chimeric-DC101 was added in dilution buffer (1% BSA, 1% goat serum, 0.01% Tween20 in PBS) and plates were incubated at room temperature for 3 hours. After three times washing, goat anti-human HRP (1:5000 diluted) was added and plates were incubated at room temperature for 1 hour. Finally, after three times washing, TMB was added and 4 mins later 0.16 mol/L sulfuric acid was added to terminate the reaction. OD450 nm was read and recorded.

As shown in FIG. 19, three lots of chimeric-DC101 had very consistent binding curve and EC₅₀, around 0.06 μg/mL, which was also quite similar to EC₅₀ of humanized 54 antibodies and 48 antibodies.

ELISA blocking assay was conducted to confirm its VEGF blockade activity. Briefly, ELISA plates were coated with 250 ng/mL recombinant human VEGF165 in coating buffer and incubated at 4° C. overnight. After twice washing, blocking buffer was added and plates were incubated at room temperature for 2 hours. After three times washing, serial diluted chimeric-DC101 was added in dilution buffer and then 1000 ng/mL mouse VEGFR2 was added. Plates were incubated at room temperature for 3 hours. After three times washing, polyclonal anti-mouse VEGFR2 with mouse Fc (1: 1000) was added and plates were incubated at room temperature for 1 hour. After three times washing, goat anti-mouse HRP (1:5000 diluted) was added and plates were incubated at room temperature for 1 hour. Finally, after three times washing, TMB was added and 4 mins later 0.16 mol/L sulfuric acid was added to terminate the reaction. OD450 nm was read and recorded.

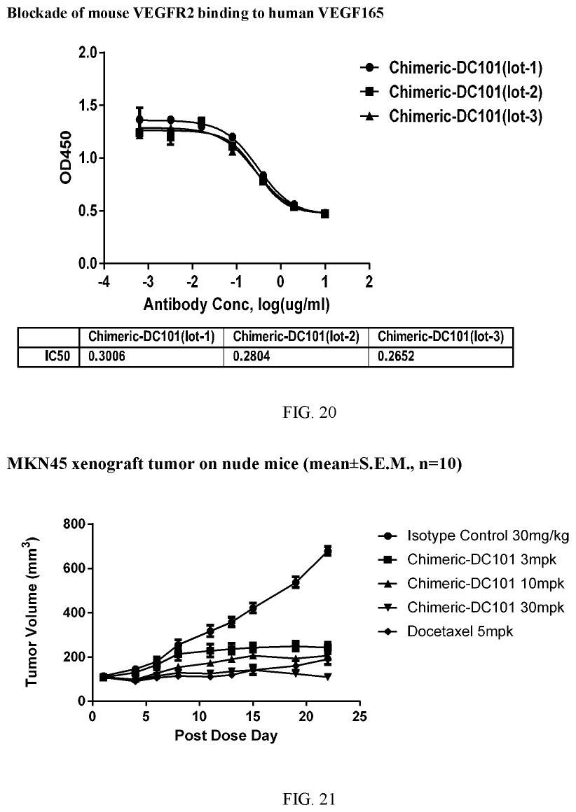

As shown in FIG. 20, three lots of chimeric-DC101 had consistent blocking curves and IC₅₀ values, which were quite similar to those of humanized anti-human VEGFR2 antibodies 054 and 048. Chimeric-DC101 was shown to have similar binding affinity and blocking activity with those of humanized 054 and 048 antibodies, indicating it can be used as surrogated antibody of 054 and 048 antibodies.

### 2. In Vivo Evaluation of the Anti-Mouse VEGFR2 Antibody DC101 Using MKN45 Gastric Tumor Model

MKN45 cells were maintained in vitro as a monolayer culture in RPMI1640 medium (Thermo Fisher) supplemented with 10% heat inactivated fetal bovine serum (ExCell Biology), 100 U/ml penicillin and 100 ug/ml streptomycin (Hyclone) at 37° C. in an atmosphere with 5% CO₂ in air. The tumor cells were routinely sub-culture twice weekly by trypsin-EDTA treatment (Hyclone). The cells growing in an exponential growth phase were harvested and counted for tumor inoculation. Female SPF grade nude mice were inoculated with mixed 5*10∧6 MKN45 cells with 50% matrigel. When the tumor size around 100 mm∧3, tumor bearing mice were selected and randomized to 5 groups (n=10). Animals were treated with 30 mg/kg isotype control, 3 mg/kg chimeric-DC101, 10 mg/kg chimeric-DC101, 30 mg/kg chimeric-DC101 and 5 mg/kg Docetaxel as positive control, isotype control and chimeric-DC101 were administrated twice a week for 3 weeks by i.p. injection, while Docetaxel was administrated once a week for 3 weeks by i.v. injection. Tumor size was measured twice a week in two dimensions using a caliper (INSIZE) and the volume was expressed in mm∧3 using the formula: V=0.5 a*b∧2 where a and b ate the long and shirt diameters of the tumor, respectively. Results were analyzed using Prism GraphPad and expressed as mean±S.E.M. Comparisons between two groups were made by T-test, and the difference is considered significant if p is *<0.05 and **<0.01.

As shown in Table 12 and FIG. 21, 3 mg/kg chimeric-DC101 has significantly inhibited MKN45 tumor growth, compared with the isotype control group. With the increase of dose, the tumor inhibition rate also increased, from 54% to 77%, indicating the tumor growth inhibition was induced by chimeric-DC101 and dose-dependent.

TABLE 12
Tumor Growth Inhibition of chimeric-DC101 on
MKN45 xenograft tumor model on Day 22

| | Tumor size | | p value |
|---|---|---|---|
| Treatment | (mm\hat{ }3, | | vs Isotype |
| (n = 10) | mean ± S.E.M.) | TGI (%) | control |
| 30 mg/kg isotype control | 678.65 ± 20.35 | / | / |
| 3 mg/kg chimeric-DC101 | 242.70 ± 23.42 | 53.76 | 3.8*10\hat{ } − 11 |
| 10 mg/kg chimeric-DC101 | 206.69 ± 20.18 | 63.96 | 2.7*10\hat{ } − 12 |
| 30 mg/kg chimeric-DC101 | 109.35 ± 13.11 | 76.85 | 5.8*10\hat{ } − 15 |
| 5 mg/kg Docetaxel | 190.07 ± 25.21 | 69.98 | 1.2*10\hat{ } − 11 |

3. In Vivo Evaluation of the Anti-Mouse VEGFR2 Antibody DC101 Using H460 Lung Tumor Model

H460 cells were maintained in vitro as a monolayer culture in RPMI1640 medium (Thermo Fisher) supplemented with 10% heat inactivated fetal bovine serum (ExCell Biology), 100 U/ml penicillin and 100 ug/ml streptomycin (Hyclone) at 37° C. in an atmosphere with 5% CO2 in air. The tumor cells were routinely sub-culture twice weekly by trypsin-EDTA treatment (Hyclone). The cells growing in an exponential growth phase were harvested and counted for tumor inoculation. Female SPF grade nude mice were inoculated with mixed 5*10∧6 H460 cells with 50% matri-gel. When the tumor size around 150 mm∧3, tumor bearing mice were selected and randomized to 5 groups (n=10). Animals were treated with 30 mg/kg isotype control, 3 mg/kg chimeric-DC101, 10 mg/kg chimeric-DC101, 30 mg/kg chimeric-DC101 and 5 mg/kg Docetaxel as positive control, isotype control and chimeric-DC101 were administrated twice a week for 2 weeks by i.p. injection, while Docetaxel was administrated once in 5 days for 3 doses by i.v. injection. Tumor size was measured twice a week in two dimensions using a caliper (INSIZE) and the volume was expressed in mm∧3 using the formula: V=0.5 a*b∧2 where a and b ate the long and shirt diameters of the tumor, respectively. Results were analyzed using Prism GraphPad and expressed as mean±S.E.M. Comparisons between two groups were made by T-test, and the difference is considered significant if p is *<0.05 and **<0.01.

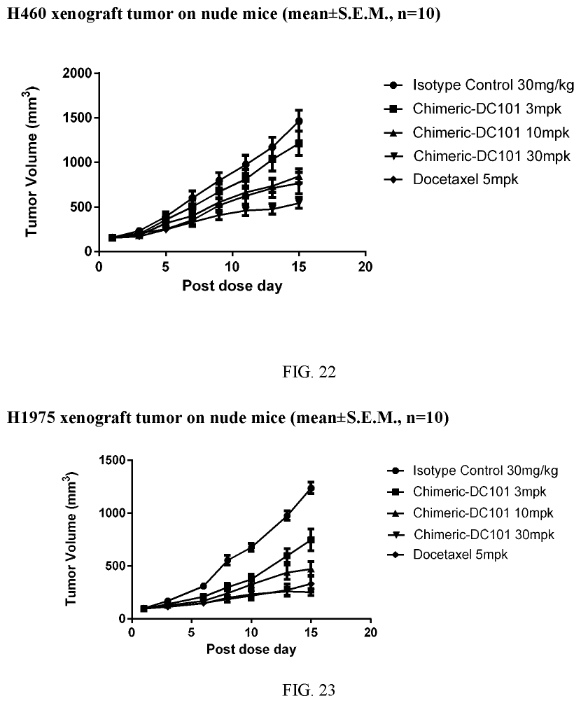

As shown in Table 13 and FIG. 22, 3 mg/kg chimeric-DC101, as the lowest dose, seemed to be not enough for inhibiting H460 tumor growth. When the dose reached 10 mg/kg and 30 mg/kg, H460 growth was significantly affected. The dose-dependent efficacy was obvious, indicating it was caused by chimeric-DC101.

TABLE 13
Tumor Growth Inhibition of chimeric-DC101 on
H460 xenograft tumor model on Day 15

| | Tumor size | | p value |
|---|---|---|---|
| Treatment | (mm\hat{ }3, | | vs Isotype |
| (n = 10) | mean ± S.E.M.) | TGI (%) | control |
| 30 mg/kg isotype control | 1463.91 ± 118.72 | / | / |
| 3 mg/kg chimeric-DC101 | 1212.70 ± 136.42 | 17.16 | 0.1817 |
| 10 mg/kg chimeric-DC101 | 845.98 ± 77.85 | 42.21 | 0.0004 |
| 30 mg/kg chimeric-DC101 | 541.96 ± 61.33 | 62.98 | 1.9*10\hat{ } − 6 |
| 5 mg/kg Docetaxel | 765.28 ± 120.64 | 47.72 | 0.0006 |

### 4. In Vivo Evaluation of the Anti-Mouse VEGFR2 Antibody DC101 Using H1975 NSCLC Tumor Model

H1975 cells were maintained in vitro as a monolayer culture in RPMI1640 medium (Thermo Fisher) supplemented with 10% heat inactivated fetal bovine serum (ExCell Biology), 100 U/ml penicillin and 100 ug/ml streptomycin (Hyclone) at 37° C. in an atmosphere with 5% CO₂ in air. The tumor cells were routinely sub-culture twice weekly by trypsin-EDTA treatment (Hyclone). The cells growing in an exponential growth phase were harvested and counted for tumor inoculation. Female SPF grade nude mice were inoculated with mixed 5*10∧6 H1975 cells with 50% matri-gel. When the tumor size around 100 mm∧3, tumor bearing mice were selected and randomized to 5 groups (n=10). Animals were treated with 30 mg/kg isotype control, 3 mg/kg chimeric-DC101, 10 mg/kg chimeric-DC101, 30 mg/kg chimeric-DC101 and 5 mg/kg Docetaxel as positive control, isotype control and chimeric-DC101 were administrated twice a week for 2 weeks by i.p. injection, while Docetaxel was administrated once a week for 2 weeks by i.v. injection. Tumor size was measured twice a week in two dimensions using a caliper (INSIZE) and the volume was expressed in mm∧3 using the formula: V=0.5 a*b∧2 where a and b ate the long and shirt diameters of the tumor, respectively. Results were analyzed using Prism GraphPad and expressed as mean±S.E.M. Comparisons between two groups were made by T-test, and the difference is considered significant if p is *<0.05 and **<0.01.

As shown in Table 14 and FIG. 23, chimeric-DC101 has significant tumor inhibition effect on H1975 xenograft tumor model, which belongs to NSCLC adenocarcinoma. With the increase of dose, from 3 mg/kg to 30 mg/kg, the tumor inhibition was more and more significant, from 40% to 80%. The dose-dependent effect indicated that the inhibition was caused by chimeric-DC101.

TABLE 14
Tumor Growth Inhibition of chimeric-DC101 on
H1975 xenograft tumor model on Day 15

| | Tumor size | | p value |
|---|---|---|---|
| Treatment | (mm\hat{ }3, | | vs Isotype |
| (n = 10) | mean ± S.E.M.) | TGI (%) | control |
| 30 mg/kg isotype control | 1237.37 ± 54.49 | | |
| 3 mg/kg chimeric-DC101 | 745.90 ± 102.69 | 39.72 | 5.1*10\hat{ } − 3 |
| 10 mg/kg chimeric-DC101 | 470.00 ± 69.44 | 62.02 | 7.4*10\hat{ } − 8 |
| 30 mg/kg chimeric-DC101 | 253.72 ± 33.94 | 79.50 | 9.0*10\hat{ } − 12 |
| 5 mg/kg Docetaxel | 330.54 ± 78.70 | 73.29 | 2.0*10\hat{ } − 8 |

1. An anti-hVEGFR antibody or an antigen-binding fragment thereof, comprising heavy chain HCDR1, HCDR2 and HCDR3 and/or light chain LCDR1, LCDR2 and LCDR3 sequences, wherein:
the HCDR1 sequence comprises SSWMN (SEQ ID NO: 1), DYYMS (SEQ ID NO: 19), X₁YGMS (SEQ ID NO: 41), X₄YWIM (SEQ ID NO: 44);
the HCDR2 sequence comprises RIFPGDGDTYYNGKFQV (SEQ ID NO: 2), FIRNKANGYTTEYSASVKG (SEQ ID NO: 20), SISX₂GGSYTYYADSVX₁₉G (SEQ ID NO: 42), DIYPGX₅GSTNYNEKFKS (SEQ ID NO: 45);
the HCDR3 sequence comprises FLDTSGRYVDY (SEQ ID NO: 3), FDYYGSTYCFDY (SEQ ID NO: 21), EX₃DGNYDY (SEQ ID NO: 43), DSNPDY (SEQ ID NO: 46);
the LCDR1 sequence comprises KASQDVNTAVA (SEQ ID NO: 4), RASQSVSTSSSSFMH (SEQ ID NO: 22), RSSKSLLYKDGKTYLN (SEQ ID NO: 28), RASESVX₆NSGISFMX₇ (SEQ ID NO: 47);
the LCDR2 sequence comprises SASYRYI (SEQ ID NO: 5), YASNLES (SEQ ID NO: 23), LMSTRAS (SEQ ID NO: 29), AASX₈QX₉S (SEQ ID NO: 48);
the LCDR3 sequence comprises QQHYRAPLT (SEQ ID NO: 6), QHTWEIPLT (SEQ ID NO: 24), QQLVEYPFT (SEQ ID NO: 30), QQSKEVPYT (SEQ ID NO: 49);
wherein X₁ is I or M, X₂ is V or I, X₃ is L or M, X₄ is T or S, X₅ is T or S, X₆ is D or E, X₇ is T or H, X₈ is T or Y, X₉ is G or R, and X₁₉ is E or K.

2. The anti-hVEGFR antibody or an antigen-binding fragment thereof of claim 1, wherein the HCDR1 comprises an amino acid sequence of SEQ ID NO: 41, the HCDR2 comprises an amino acid sequence of SEQ ID NO: 42, the HCDR3 comprises an amino acid sequence of SEQ ID NO: 43, the LCDR1 comprises a sequence of SEQ ID NO: 28, the LCDR2 comprises a sequence of SEQ ID NO: 29, and the LCDR3 comprises a sequence of SEQ ID NO: 30.

3. The antibody or an antigen-binding fragment thereof of claim 2, wherein
a) the HCDR1 comprises the sequence of SEQ ID NO: 25, a HCDR2 comprises the sequence of SEQ ID NO: 26, the HCDR3 comprises the sequence of SEQ ID NO: 27; the LCDR1 comprises the sequence of SEQ ID NO: 28, the LCDR2 comprises the sequence of SEQ ID NO: 29, and the LCDR3 comprises the sequence of SEQ ID NO: 30; or
b) the HCDR1 comprises the sequence of SEQ ID NO: 31, the HCDR2 comprises the sequence of SEQ ID NO: 32 or SEQ ID NO: 37, and the HCDR3 comprises the sequence of SEQ ID NO: 33, the LCDR1 comprises the sequence of SEQ ID NO: 28, the LCDR2 comprises the sequence of SEQ ID NO: 29, and the LCDR3 comprises the sequence of SEQ ID NO: 30; or
c) the HCDR1 comprises the sequence of SEQ ID NO: 34, the HCDR2 comprises the sequence of SEQ ID NO: 35 or SEQ ID NO: 37, and the HCDR3 comprises the sequence of SEQ ID NO: 36, the LCDR1 comprises the sequence of SEQ ID NO: 28, the LCDR2 comprises the sequence of SEQ ID NO: 29, and the LCDR3 comprises the sequence of SEQ ID NO: 30.

4. The anti-hVEGFR antibody or an antigen-binding fragment thereof of claim 1, wherein the HCDR1 comprises an amino acid sequence of SEQ ID NO: 44, the HCDR2 comprises an amino acid sequence of SEQ ID NO: 45, the HCDR3 comprises an amino acid sequence of SEQ ID NO: 46, the LCDR1 comprises a sequence of SEQ ID NO: 47, the LCDR2 comprises a sequence of SEQ ID NO: 48, and the LCDR3 comprises a sequence of SEQ ID NO: 49.

5. The antibody or an antigen-binding fragment thereof of claim 4, wherein
a) the HCDR1 comprises the sequence of SEQ ID NO: 7, a HCDR2 comprises the sequence of SEQ ID NO: 8, the HCDR3 comprises the sequence of SEQ ID NO:
9; the LCDR1 comprises the sequence of SEQ ID NO: 10, the LCDR2 comprises the sequence of SEQ ID NO: 11, and the LCDR3 comprises the sequence of SEQ ID NO: 12; or
b) the HCDR1 comprises the sequence of SEQ ID NO: 13, the HCDR2 comprises the sequence of SEQ ID NO: 14, and the HCDR3 comprises the sequence of SEQ ID NO: 15, the LCDR1 comprises the sequence of SEQ ID NO: 16, the LCDR2 comprises the sequence of SEQ ID NO: 17, and the LCDR3 comprises the sequence of SEQ ID NO: 18.

6. The antibody or an antigen-binding fragment thereof of claim 1, wherein
a) the HCDR1 comprises the sequence of SEQ ID NO: 1, the HCDR2 comprises the sequence of SEQ ID NO: 2, and the HCDR3 comprises the sequence of SEQ ID NO: 3, the LCDR1 comprises the sequence of SEQ ID NO: 4, the LCDR2 comprises the sequence of SEQ ID NO: 5, and the LCDR3 comprises the sequence of SEQ ID NO: 6; or
b) the HCDR1 comprises the sequence of SEQ ID NO: 19, the HCDR2 comprises the sequence of SEQ ID NO: 20, and the HCDR3 comprises the sequence of SEQ ID NO: 21, the LCDR1 comprises the sequence of SEQ ID NO: 22, the LCDR2 comprises the sequence of SEQ ID NO: 23, and the LCDR3 comprises the sequence of SEQ ID NO: 24.

7. (canceled)

8. The antibody or antigen-binding fragment thereof of claim 7, wherein:
the HFR1 comprises a sequence selected from the group consisting of SEQ ID NOs: 64, 68 and 72,
the HFR2 comprises a sequence selected from the group consisting of SEQ ID NOs: 65, 69 and 73,
the HFR3 comprises the sequence selected from the group consisting of SEQ ID NOs: 66, 70 and 74,
the HFR4 comprises a sequence selected from the group consisting of SEQ ID NOs: 67, 71 and 75,
the LFR1 comprises the sequence selected from the group consisting of SEQ ID NOs: 76 and 80,
the LFR2 comprises a sequence selected from the group consisting of SEQ ID NO: 77 and 81,
the LFR3 comprises a sequence selected from the group consisting of SEQ ID NOs: 78 and 82, and
the LFR4 comprises a sequence selected from the group consisting of SEQ ID NOs: 79 and 83.

9. The antibody or an antigen-binding fragment thereof of claim 1, wherein the heavy chain variable region comprises a sequence selected from the group consisting of SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 93, SEQ ID NO: 94, SEQ ID NO: 98, and a homologous sequence thereof having at least 80% sequence identity yet retaining specific binding affinity to hVEGFR2; and wherein the light chain variable region comprises a sequence selected from the group consisting of SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 96, SEQ ID NO: 97, and a homologous sequence thereof having at least 80% sequence identity yet retaining specific binding affinity to hVEGFR2.

10. (canceled)

11. The antibody or an antigen-binding fragment thereof of claim 1, wherein:
a) a heavy chain variable region comprising the sequence of SEQ ID NO: 50 and a light chain variable region comprising the sequence of SEQ ID NO: 51;
b) the heavy chain variable region comprises a sequence of SEQ ID NO: 52 and the light chain variable region comprises a sequence of SEQ ID NO: 53;
c) the heavy chain variable region comprises a sequence of SEQ ID NO: 54 and the light chain variable region comprises a sequence of SEQ ID NO: 55;
d) the heavy chain variable region comprises a sequence of SEQ ID NO: 56 and the light chain variable region comprises a sequence of SEQ ID NO: 57;
e) the heavy chain variable region comprises a sequence of SEQ ID NO: 58 and the light chain variable region comprises a sequence of SEQ ID NO: 59;
f) the heavy chain variable region comprises a sequence of SEQ ID NO: 60 and the light chain variable region comprises a sequence of SEQ ID NO: 61;
g) the heavy chain variable region comprises a sequence of SEQ ID NO: 62 and the light chain variable region comprises a sequence of SEQ ID NO: 63; or
h) the heavy chain variable region comprises a sequence of SEQ ID NO: 93 or SEQ ID NO: 94 or SEQ ID NO: 98 and the light chain variable region comprises a sequence of SEQ ID NO: 96 or SEQ ID NO: 97.

12-13. (canceled)

14. The antibody or antigen-binding fragment thereof of claim 1, further comprising an immunoglobulin constant region, or further comprising a constant region of human Ig, or further comprising a constant region of human IgG.

15. (canceled)

16. The antibody or antigen-binding fragment thereof of claim 15, wherein the constant region of human IgG is human IgG1 comprising SEQ ID NO: 38, or a homologous sequence having at least 80% sequence identity thereof.

17. The antibody or an antigen-binding fragment thereof of claim 1, which is humanized.

18. The antibody or antigen-binding fragment thereof of claim 1, which is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

19. The antibody or antigen-binding fragment thereof of claim 1, which is bispecific, capable of specifically binding to a first and a second epitope of hVEGFR2, or capable of specifically binding to hVEGFR2 and a second antigen.

20. (canceled)

21. The antibody or antigen-binding fragment thereof of claim 19, wherein the second antigen is an immune related target or a tumor antigen, wherein the immune related target is selected from the group consisting of PD-L1, PD-L2, PD-1, CLTA-4, TIM-3, LAG3, CD160, 2B4, TGF β, VISTA, BTLA, TIGIT, LAIR1, OX40, CD2, CD27, ICAM-1, NKG2C, SLAMF7, NKp80, CD160, B7-H3, LFA-1, 1COS, 4-1BB, GITR, CD30, CD40, BAFFR, HVEM, CD7, LIGHT, IL-2, IL-15, CD3, CD16 and CD83, and wherein the tumor antigen is selected from the group consisting of, claudin 18.2, CA-125, gangliosides G(D2), G(M2) and G(D3), CD20, CD52, CD33, Ep-CAM, CEA bombesin-like peptides, PSA, HER2/neu, epidermal growth factor receptor (EGFR), erbB2, erB3/HER3, erbB4, CD44v6, Ki-67, cancer-associated mucin, VEGF, VEGFRs (e.g., VEGFR3), estrogen receptors, Lewis-Y antigen, TGFβ1, IGF-1 receptor, EGFα, c-Kit receptor, transferrin receptor, IL-2R and CO17-1A.

22-24. (canceled)

25. The antibody or antigen-binding fragment thereof of claim 1 linked to one or more conjugate moieties, wherein the conjugate moiety comprises a clearance-modifying agent, a chemotherapeutic agent, a toxin, a radioactive isotope, a lanthanide, a luminescent label, a fluorescent label, an enzyme-substrate label, a DNA-alkylators, a topoisomerase inhibitor, a tubulin-binders, or other anticancer drugs.

26-27. (canceled)

28. A pharmaceutical composition comprising the antibody or antigen-binding fragment thereof of claim 1, and one or more pharmaceutically acceptable carriers.

29-30. (canceled)

31. An isolated polynucleotide encoding the antibody or an antigen-binding fragment thereof of claim 1.

32. A vector comprising the isolated polynucleotide of claim 31.

33. A host cell comprising the vector of claim 32.

34. A method of expressing the antibody or antigen-binding fragment thereof of claim 1, comprising culturing a host cell comprising a vector comprising an isolated polynucleotide encoding the antibody o antigen-binding fragment thereof of claim 1 under the condition at which the vector is expressed.

35. A method of treating, reducing the severity of and/or slowing the progression of a VEGFR2-related disease or condition in a subject, comprising administering to the subject a therapeutically effective amount of the antibody or antigen-binding fragment thereof of claim 1.

36. The method of claim 35, wherein the VEGFR2-related disease or condition is a tumor or an angiogenic disease.

37. The method of claim 36, wherein the tumor produces VEGF (e.g., VEGF-A) and/or is sensitive to VEGF (e.g., VEGF-A) present in its microenvironment.

38. (canceled)

39. The method of claim 36, wherein the tumor is selected from the group consisting of breast carcinoma, lung carcinoma, colorectal carcinoma, pancreatic carcinoma, glioma and lymphoma, head and neck tumors, neuroendocrine tumors, colorectal tumors, prostate tumors, breast tumors, lung tumors, such as small cell and non-small cell lung tumors, pancreatic tumors, thyroid tumors, ovarian tumors, liver tumors, Kaposi's sarcoma, CNS neoplasms, neuroblastomas, capillary hemangioblastomas, meningiomas, cerebral metastases, melanoma, gastrointestinal and renal carcinomas and sarcomas (e.g., gastric cancer), rhabdomyosarcoma, glioblastoma, preferably glioblastoma multiforme, leiomyosarcoma, squamous cell carcinoma, basal cell carcinoma and skin cancers that can be treated by suppressing the growth of malignant keratinocytes, such as human malignant keratinocytes, and wherein the angiogenetic disease is selected from the group consisting of atherosclerosis, rheumatoid arthritis (RA), neovascular glaucoma, proliferative retinopathy including proliferative diabetic retinopathy, macular degeneration, hemangiomas, angiofibromas, psoriasis, retinopathy of prematurity (e.g., retrolental fibroplastic), corneal graft rejection, insulin-dependent diabetes mellitus, multiple sclerosis, myasthenia gravis, Chron's disease, autoimmune nephritis, primary biliary cirrhosis, acute pancreatitis, allograph rejection, allergic inflammation, contact dermatitis, and delayed hypersensitivity reactions, inflammatory bowel disease, septic shock, osteoporosis, osteoarthritis, cognition defects induced by neuronal inflammation, Osler-Weber syndrome, restinosis, and fungal, parasitic and viral infections, such as cytomegaloviral infections.

40-42. (canceled)

43. The method of claim 35, wherein the subject is human.

44-47. (canceled)

48. A method of detecting presence or amount of VEGFR2 in a sample, comprising contacting the sample with the antibody or antigen-binding fragment thereof of claim 1, and determining the presence or the amount of VEGFR2 in the sample.

49-53. (canceled)


SEQUENCE LISTING (103 sequences)

SEQ ID NO: 1 - protein, 5 aa, Artificial Sequence
  Synthetic
SSWMN

SEQ ID NO: 2 - protein, 17 aa, Artificial Sequence
  Synthetic
RIFPGDGDTYYNGKFQV

SEQ ID NO: 3 - protein, 11 aa, Artificial Sequence
  Synthetic
FLDTSGRYVDY

SEQ ID NO: 4 - protein, 11 aa, Artificial Sequence
  Synthetic
KASQDVNTAVA

SEQ ID NO: 5 - protein, 7 aa, Artificial Sequence
  Synthetic
SASYRYI

SEQ ID NO: 6 - protein, 9 aa, Artificial Sequence
  Synthetic
QQHYRAPLT

SEQ ID NO: 7 - protein, 5 aa, Artificial Sequence
  Synthetic
TYWIM

SEQ ID NO: 8 - protein, 17 aa, Artificial Sequence
  Synthetic
DIYPGTGSTNYNEKFKS

SEQ ID NO: 9 - protein, 6 aa, Artificial Sequence
  Synthetic
DSNPDY

SEQ ID NO: 10 - protein, 15 aa, Artificial Sequence
  Synthetic
RASESVDNSGISFMT

SEQ ID NO: 11 - protein, 7 aa, Artificial Sequence
  Synthetic
AASTQGS

SEQ ID NO: 12 - protein, 9 aa, Artificial Sequence
  Synthetic
QQSKEVPYT

SEQ ID NO: 13 - protein, 5 aa, Artificial Sequence
  Synthetic
SYWIM

SEQ ID NO: 14 - protein, 17 aa, Artificial Sequence
  Synthetic
DIYPGSGSTNYNEKFKS

SEQ ID NO: 15 - protein, 6 aa, Artificial Sequence
  Synthetic
DSNPDY

SEQ ID NO: 16 - protein, 15 aa, Artificial Sequence
  Synthetic
RASESVENSGISFMH

SEQ ID NO: 17 - protein, 7 aa, Artificial Sequence
  Synthetic
AASYQRS

SEQ ID NO: 18 - protein, 9 aa, Artificial Sequence
  Synthetic
QQSKEVPYT

SEQ ID NO: 19 - protein, 5 aa, Artificial Sequence
  Synthetic
DYYMS

SEQ ID NO: 20 - protein, 19 aa, Artificial Sequence
  Synthetic
FIRNKANGYTTEYSASVKG

SEQ ID NO: 21 - protein, 12 aa, Artificial Sequence
  Synthetic
FDYYGSTYCFDY

SEQ ID NO: 22 - protein, 15 aa, Artificial Sequence
  Synthetic
RASQSVSTSSSSFMH

SEQ ID NO: 23 - protein, 7 aa, Artificial Sequence
  Synthetic
YASNLES

SEQ ID NO: 24 - protein, 9 aa, Artificial Sequence
  Synthetic
QHTWEIPLT

SEQ ID NO: 25 - protein, 5 aa, Artificial Sequence
  Synthetic
IYGMS

SEQ ID NO: 26 - protein, 17 aa, Artificial Sequence
  Synthetic
SISVGGSYTYYADSVEG

SEQ ID NO: 27 - protein, 8 aa, Artificial Sequence
  Synthetic
ELDGNYDY

SEQ ID NO: 28 - protein, 16 aa, Artificial Sequence
  Synthetic
RSSKSLLYKDGKTYLN

SEQ ID NO: 29 - protein, 7 aa, Artificial Sequence
  Synthetic
LMSTRAS

SEQ ID NO: 30 - protein, 9 aa, Artificial Sequence
  Synthetic
QQLVEYPFT

SEQ ID NO: 31 - protein, 5 aa, Artificial Sequence
  Synthetic
MYGMS

SEQ ID NO: 32 - protein, 17 aa, Artificial Sequence
  Synthetic
SISIGGSYTYYADSVEG

SEQ ID NO: 33 - protein, 8 aa, Artificial Sequence
  Synthetic
EMDGNYDY

SEQ ID NO: 34 - protein, 5 aa, Artificial Sequence
  Synthetic
MYGMS

SEQ ID NO: 35 - protein, 17 aa, Artificial Sequence
  Synthetic
SISIGGSYTYYADSVEG

SEQ ID NO: 36 - protein, 8 aa, Artificial Sequence
  Synthetic
ELDGNYDY

SEQ ID NO: 37 - protein, 17 aa, Artificial Sequence
  Synthetic
SISIGGSYTYYADSVKG

SEQ ID NO: 38 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 1356 aa, Artificial Sequence [sequence omitted]

SEQ ID NO: 40 - protein, 470 aa, Artificial Sequence
  Synthetic
MEFGLSWLFLVAILKGVQCEVQLVETGGGLVQPGNSLKLSCATSGFIFSTTWMNWIRQTPGKRLEWLAQIEDKSNNYFISYSESVKGRFTISRDDSKSSVYLQMNNLKEEDTAIYYCSWKYRSNYYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 5 aa, Artificial Sequence
  Synthetic
  X (1)..(1): I or M
XYGMS

SEQ ID NO: 42 - protein, 17 aa, Artificial Sequence
  Synthetic
  X (4)..(4): V or I
  X (16)..(16): E or K
SISXGGSYTYYADSVXG

SEQ ID NO: 43 - protein, 8 aa, Artificial Sequence
  Synthetic
  X (2)..(2): L or M
EXDGNYDY

SEQ ID NO: 44 - protein, 5 aa, Artificial Sequence
  Synthetic
  X (1)..(1): T or S
XYWIM

SEQ ID NO: 45 - protein, 17 aa, Artificial Sequence
  Synthetic
  X (6)..(6): T or S
DIYPGXGSTNYNEKFKS

SEQ ID NO: 46 - protein, 6 aa, Artificial Sequence
  Synthetic
DSNPDY

SEQ ID NO: 47 - protein, 15 aa, Artificial Sequence
  Synthetic
  X (7)..(7): D or E
  X (15)..(15): T or H
RASESVXNSGISFMX

SEQ ID NO: 48 - protein, 7 aa, Artificial Sequence
  Synthetic
  X (4)..(4): T or Y
  X (6)..(6): G or R
AASXQXS

SEQ ID NO: 49 - protein, 9 aa, Artificial Sequence
  Synthetic
QQSKEVPYT

SEQ ID NO: 50 - protein, 120 aa, Artificial Sequence
  Synthetic
QVQLQQSGPELVKPGASVKISCKASGYAFSSSWMNWVKQRPGEGLEWIGRIFPGDGDTYYNGKFQVKATLTADKSSSTAYMQLSSLTSEDSAVYFCAIFLDTSGRYVDYWGQGTTLTISS

SEQ ID NO: 51 - protein, 107 aa, Artificial Sequence
  Synthetic
DIVMTQSHKFMSTSVGDRVSITCKASQDVNTAVAWYQQKPGQSPKLLIYSASYRYIGVPDRFTGSGSGTDFTFTISSVQSEDLTVYYCQQHYRAPLTFGSGTKLELK

SEQ ID NO: 52 - protein, 115 aa, Artificial Sequence
  Synthetic
QVQLQQPGAELVKPGASVKMSCKASGYTFNTYWIMWVKQRPGQGLEWIGDIYPGTGSTNYNEKFKSKVTLTADTSSTTAYMQVSSLTSEDSAVYYCGRDSNPDYWGQGTTLTVSS

SEQ ID NO: 53 - protein, 111 aa, Artificial Sequence
  Synthetic
DIVLTQSPASLAVSLGQRATISCRASESVDNSGISFMTWFQQKPGQPPKLLIYAASTQGSGVPARFSGSGSGTDFSLNIHPVEEDDTAMYFCQQSKEVPYTFGGGTKLEIK

SEQ ID NO: 54 - protein, 115 aa, Artificial Sequence
  Synthetic
QAQLQQPGAELVKPGTSVKMSCKASGYTFNSYWIMWVKQSPGQGLEWIGDIYPGSGSTNYNEKFKSKVTLTVDTSSSTAYMQVSSLTSEDSAVYYCARDSNPDYWGQGTTLTVSS

SEQ ID NO: 55 - protein, 111 aa, Artificial Sequence
  Synthetic
DIVLTQSPASLAVSLGQRATISCRASESVENSGISFMHWFQQKPGQPPKLLIYAASYQRSGVPARFSGSGSGTDFSLNIHPVEEDDIAMYFCQQSKEVPYTFGGGTKLEIK

SEQ ID NO: 56 - protein, 123 aa, Artificial Sequence
  Synthetic
EVKLVESGGGLVQPGGSLSLSCAVSGFTFTDYYMSWVRQPPGKALEWLGFIRNKANGYTTEYSASVKGRFTISRDNSQSILYLQMNALRAEDSATYYCARFDYYGSTYCFDYWGQGTTLTVSS

SEQ ID NO: 57 - protein, 111 aa, Artificial Sequence
  Synthetic
DIVLTQSPASLAVSLGQRATISCRASQSVSTSSSSFMHWYQQKPGQPPKLLIKYASNLESGVPARFSGSGSGTDFTLNIHPVEEEDTATYYCQHTWEIPLTFGAGTKLELK

SEQ ID NO: 58 - protein, 117 aa, Artificial Sequence
  Synthetic
EVQLVESGGDLVKPGGSLKLSCTASGFSFSIYGMSWVRQTPDKRLEWVASISVGGSYTYYADSVEGRFTISRENAKNTLYLQMNSLKSEDTALYYCARELDGNYDYWGQGTSLTVSS

SEQ ID NO: 59 - protein, 112 aa, Artificial Sequence
  Synthetic
DIVITQNELSNPVTFGESVSISCRSSKSLLYKDGKTYLNWFLQRPGQSPQLLIYLMSTRASGVSDRFSGSGSGTDFTLEISRVKAEDVGVYYCQQLVEYPFTFGSGTKLEIK

SEQ ID NO: 60 - protein, 117 aa, Artificial Sequence
  Synthetic
EVQLVESGGDLVKPGGSLKLSCAASGFTFSMYGMSWVRQTPDKRLEWVASISIGGSYTYYADSVEGRFTISRENAKNTLFLQMNSLKSEDTALYYCAREMDGNYDYWGHGTTLTVSS

SEQ ID NO: 61 - protein, 112 aa, Artificial Sequence
  Synthetic
DVMITQDELSNPVTFGESVSISCRSSKSLLYKDGKTYLNWFLQRPGQSPQLLIYLMSTRASGVSDRFSGSGSGTDFTLEISRVKAEDVGIYYCQQLVEYPFTFGSGTKLEIK

SEQ ID NO: 62 - protein, 117 aa, Artificial Sequence
  Synthetic
EVQLVESGGDLVKPGGSLKLSCAASGFTFSMYGMSWVRQTPDKRLEWVASISIGGSYTYYADSVEGRFIISRENAKNTLFLQMNSLKSEDTALYYCARELDGNYDYWGQGTTLTVSS

SEQ ID NO: 63 - protein, 112 aa, Artificial Sequence
  Synthetic
HIMITQDELSNPVTFGESVSISCRSSKSLLYKDGKTYLNWFLQRPGQSPQLLIYLMSTRASGVSDRFSGSGSGTDFTLEISRVKAEDVGIYYCQQLVEYPFTFGSGTKLEIK

SEQ ID NO: 64 - protein, 30 aa, Artificial Sequence
  Synthetic
EVQLVESGGGLVKPGGSLKLSCAASGFTFS

SEQ ID NO: 65 - protein, 14 aa, Artificial Sequence
  Synthetic
WVRQTPGKRLEWVA

SEQ ID NO: 66 - protein, 32 aa, Artificial Sequence
  Synthetic
RFTISRDNAKNTLYLQMNSLKAEDTAVYYCAR

SEQ ID NO: 67 - protein, 11 aa, Artificial Sequence
  Synthetic
WGQGTTLTVSS

SEQ ID NO: 68 - protein, 30 aa, Artificial Sequence
  Synthetic
EVQLVESGGGLVKPGGSLRLSCAASGFTFS

SEQ ID NO: 69 - protein, 14 aa, Artificial Sequence
  Synthetic
WVRQAPGKRLEWVA

SEQ ID NO: 70 - protein, 32 aa, Artificial Sequence
  Synthetic
RFTISRDNAKNTLYLQMNSLRAEDTAVYYCAR

SEQ ID NO: 71 - protein, 11 aa, Artificial Sequence
  Synthetic
WGQGTTLTVSS

SEQ ID NO: 72 - protein, 30 aa, Artificial Sequence; identical to SEQ ID NO: 68
  Synthetic

SEQ ID NO: 73 - protein, 14 aa, Artificial Sequence
  Synthetic
WVRQAPGKRLEWVA

SEQ ID NO: 74 - protein, 32 aa, Artificial Sequence; identical to SEQ ID NO: 70
  Synthetic

SEQ ID NO: 75 - protein, 11 aa, Artificial Sequence
  Synthetic
WGHGTTLTVSS

SEQ ID NO: 76 - protein, 23 aa, Artificial Sequence
  Synthetic
DIVITQDELSLPVTFGESVSISC

SEQ ID NO: 77 - protein, 15 aa, Artificial Sequence
  Synthetic
WFLQRPGQSPQLLIY

SEQ ID NO: 78 - protein, 32 aa, Artificial Sequence
  Synthetic
GVSDRFSGSGSGTDFTLKISRVEAEDVGIYYC

SEQ ID NO: 79 - protein, 10 aa, Artificial Sequence
  Synthetic
FGSGTKLEIK

SEQ ID NO: 80 - protein, 23 aa, Artificial Sequence
  Synthetic
DIVITQTPLSLPVTPGESVSISC

SEQ ID NO: 81 - protein, 15 aa, Artificial Sequence
  Synthetic
WFLQRPGQSPQLLIY

SEQ ID NO: 82 - protein, 32 aa, Artificial Sequence
  Synthetic
GVPDRFSGSGSGTDFTLKISRVEAEDVGVYYC

SEQ ID NO: 83 - protein, 10 aa, Artificial Sequence
  Synthetic
FGSGTKLEIK

SEQ ID NO: 84 - protein, 30 aa, Artificial Sequence
  Synthetic
  X (19)..(19): R or K
EVQLVESGGGLVKPGGSLXLSCAASGFTFS

SEQ ID NO: 85 - protein, 14 aa, Artificial Sequence
  Synthetic
  X (5)..(5): A or T
WVRQXPGKRLEWVA

SEQ ID NO: 86 - protein, 32 aa, Artificial Sequence
  Synthetic
  X (21)..(21): R or K
RFTISRDNAKNTLYLQMNSLXAEDTAVYYCAR

SEQ ID NO: 87 - protein, 11 aa, Artificial Sequence
  Synthetic
  X (3)..(3): Q or H
WGXGTTLTVSS

SEQ ID NO: 88 - protein, 23 aa, Artificial Sequence
  Synthetic
  X (7)..(7): D or T
  X (8)..(8): E or P
  X (15)..(15): F or P
DIVITQXXLSLPVTXGESVSISC

SEQ ID NO: 89 - protein, 15 aa, Artificial Sequence
  Synthetic
WFLQRPGQSPQLLIY

SEQ ID NO: 90 - protein, 32 aa, Artificial Sequence
  Synthetic
  X (3)..(3): S or P
  X (29)..(29): V or I
GVXDRFSGSGSGTDFTLKISRVEAEDVGXYYC

SEQ ID NO: 91 - protein, 10 aa, Artificial Sequence
  Synthetic
FGSGTKLEIK

SEQ ID NO: 92 - protein, 117 aa, Artificial Sequence
  Synthetic
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARELDGNYDYWGQGTTLTVSS

SEQ ID NO: 93 - protein, 117 aa, Artificial Sequence
  Synthetic
EVQLVESGGGLVKPGGSLKLSCAASGFTFSMYGMSWVRQTPGKRLEWVASISIGGSYTYYADSVKGRFTISRDNAKNTLYLQMNSLKAEDTAVYYCARELDGNYDYWGQGTTLTVSS

SEQ ID NO: 94 - protein, 117 aa, Artificial Sequence
  Synthetic
EVQLVESGGGLVKPGGSLRLSCAASGFTFSMYGMSWVRQAPGKRLEWVASISIGGSYTYYADSVKGRFTISRDNAKNTLYLQMNSLRAEDTAVYYCARELDGNYDYWGQGTTLTVSS

SEQ ID NO: 95 - protein, 101 aa, Artificial Sequence
  Synthetic
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFP

SEQ ID NO: 96 - protein, 112 aa, Artificial Sequence
  Synthetic
DIVITQDELSLPVTFGESVSISCRSSKSLLYKDGKTYLNWFLQRPGQSPQLLIYLMSTRASGVSDRFSGSGSGTDFTLKISRVEAEDVGIYYCQQLVEYPFTFGSGTKLEIK

SEQ ID NO: 97 - protein, 112 aa, Artificial Sequence
  Synthetic
DIVITQTPLSLPVTPGESVSISCRSSKSLLYKDGKTYLNWFLQRPGQSPQLLIYLMSTRASGVPDRFSGSGSGTDFTLKISRVEAEDVGVYYCQQLVEYPFTFGSGTKLEIK

SEQ ID NO: 98 - protein, 117 aa, Artificial Sequence
  Synthetic
EVQLVESGGGLVKPGGSLRLSCAASGFTFSMYGMSWVRQAPGKRLEWVASISIGGSYTYYADSVKGRFTISRDNAKNTLYLQMNSLRAEDTAVYYCAREMDGNYDYWGHGTTLTVSS

SEQ ID NO: 99 - protein, 239 aa, Artificial Sequence
  Synthetic
MDMRVPAQLLGLLLLWFPGSRCDIVLTQSPALAVSLEQRATISCKTSQNVDYYGISYLHWYQQKPGQQPKLLIYEGSNLASGIPARFSGSGSGTDFTLTIDPVEADDIVTYYCQQSKDYPYTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 1367 aa, Artificial Sequence
  Synthetic
MESKALLAVALWFCVETRAASVGLPGDFLHPPKLSTQKDILTILANTTLQITCRGQRDLDWLWPNAQRDSEERVLVTECGGGDSIFCKTLTIPRVVGNDTGAYKCSYRDVDIASTVYVYVRDYRSPFIASVSDQHGIVYITENKNKTVVIPCRGSISNLNVSLCARYPEKRFVPDGNRISWDSEIGFTLPSYMISYAGMVFCEAKINDETYQSIMYIVVVVGYRIYDVILSPPHEIELSAGEKLVLNCTARTELNVGLDFTWHSPPSKSHHKKIVNRDVKPFPGTVAKMFLSTLTIESVTKSDQGEYTCVASSGRMIKRNRTFVRVHTKPFIAFGSGMKSLVEATVGSQVRIPVKYLSYPAPDIKWYRNGRPIESNYTMIVGDELTIMEVTERDAGNYTVILTNPISMEKQSHMVSLVVNVPPQIGEKALISPMDSYQYGTMQTLTCTVYANPPLHHIQWYWQLEEACSYRPGQTSPYACKEWRHVEDFQGGNKIEVTKNQYALIEGKNKTVSTLVIQAANVSALYKCEAINKAGRGERVISFHVIRGPEITVQPAAQPTEQESVSLLCTADRNTFENLTWYKLGSQATSVHMGESLTPVCKNLDALWKLNGTMFSNSTNDILIVAFQNASLQDQGDYVCSAQDKKTKKRHCLVKQLIILERMAPMITGNLENQTTTIGETIEVTCPASGNPTPHITWFKDNETLVEDSGIVLRDGNRNLTIRRVRKEDGGLYTCQACNVLGCARAETLFIIEGAQEKTNLEVIILVGTAVIAMFFWLLLVILVRTVKRANEGELKTGYLSIVMDPDELPLDERCERLPYDASKWEFPRDRLKLGKPLGRGAFGQVIEADAFGIDKTATCKTVAVKMLKEGATHSEHRALMSELKILIHIGHHLNVVNLLGACTKPGGPLMVIVEFSKFGNLSTYLRGKRNEFVPYKSKGARFRQGKDYVGELSVDLKRRLDSITSSQSSASSGFVEEKSLSDVEEEEASEELYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHEDPNQRPSFSELVEHLGNLLQANAQQDGKDYIVLPMSETLSMEEDSGLSLPTSPVSCMEEEEVCDPKFHYDNTAGISHYLQNSKRKSRPVSVKTFEDIPLEEPEVKVIPDDSQTDSGMVLASEELKTLEDRNKLSPSFGGMMPSKSRESVASEGSNQTSGYQSGYHSDDTDTTVYSSDEAGLLKMVDAAVHADSGTTLQLTSCLNGSGPVPAPPPTPGNHERGAA

SEQ ID NO: 101 - protein, 1356 aa, Artificial Sequence
  Synthetic
MASKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGVELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPVKYLGYPPPEIKWYKNGIPLESNHTVKVGHVLTIMEVSERDTGNYTVILTNPISKEKQSHVVSLVVYVPPQIGEKSLISPVDSYQYGTTQTLTCTVYAIPPPHHIHWYWQLEEECPNEPSQAVSVTNPYPCEEWRSVEDFQGGNKIEVNKNQFALIEGKNKTVSTLVIQAANVSALYKCEAVNKVGRGERVISFHVTRGPEITLQPDLQPTEQESVSLWCTADKSTFENLTWYKLGPQPLPVHVGELPTPVCKNLDTLWKLNATIFSNSTNDILIMELKNASLQDQGDYVCVAQDRKTKKRHCVVRQLTVLERVAPMITGNLENQTTSIGETIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLAPSFSGMVSSKSRESVASEGSNQTSGYQSGYHSDDTDTTVYSSEEAELLKLIEIGVQTGSTAQILQPDSGTTLSSPPV

SEQ ID NO: 102 - protein, 98 aa, Artificial Sequence
  Synthetic
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAR

SEQ ID NO: 103 - protein, 712 aa, Artificial Sequence
  Synthetic
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSE